(19) United States
(12) Patent Application Publication
(10) Pub. No.: US 2006/0257865 A1
(43) Pub. Date: Nov. 16, 2006

(54) Method for identification and development of therapeutic agents

(76) Inventor: Simon Mallal, Wembley (AU)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/493,165

(22) PCT Filed: Oct. 23, 2002
(86) PCT No.: PCT/AU02/01450

§ 371 Date: Sep. 21, 2004

(30) Foreign Application Priority Data
Oct. 23, 2001 (AU) PR 8425

(51) Int. Cl. C12Q 1/68; G01N 33/554; G06F 19/00

(52) U.S. Cl. 435/6; 435/7.32; 702/19

(57) ABSTRACT

The present invention relates generally to the field of identification and determination of bioactive amino acid sequences. In particular, the present invention provides method(s) for determining the influence of variation in host genes on selection of microorganisms with particular amino acid variants for the purpose of therapeutic drug or vaccine design or individualisation of such treatment. The invention also provides methods for identifying HLA allele-specific microorganism sequence polymorphisms that result from HLA restriction of antigen-specific cellular immune responses. It also provides diagnostic and therapeutic methodologies that may be used to measure or treat infection by a microorganism or to prevent infection by the microorganism.

### FIELD OF THE INVENTION

The present invention relates generally to the field of identification and determination of bioactive amino acid sequences. In particular, the present invention provides method(s) for determining the influence of variation in host genes on selection of microorganisms with particular amino acid variants for the purpose of therapeutic drug or vaccine design or individualisation of such treatment. The invention also provides methods for identifying HLA allele-specific microorganism sequence polymorphisms that result from HLA restriction of antigen-specific cellular immune responses. It also provides diagnostic and therapeutic methodologies that may be used to measure or treat infection by a microorganism or to prevent infection by the microorganism.

### BACKGROUND ART

An animal's response to a pathological microorganism or a tumour may be made up of a vast array of biological reactions and interactions. For example, the immune response to viral-infected cells has been shown to be mediated largely by a subpopulation of effector T lymphocytes known as CD8+ T-cells or cytotoxic T lymphocytes (CTL). Although these cells can directly kill viral-infected cells, they generally require help in the form of soluble products, or cytokines, produced by another subpopulation of T lymphocytes known as CD4+ helper T-cells.

The principle CTL receptor involved in recognition of a pathological microorganism and initiation or activation of a counteractive immune response is the antigen-specific receptor known as the T-cell receptor molecule present only on the surface of T-cells. This receptor engages specifically with a processed peptide antigen presented in the context of a Major Histocompatibility Complex (MHC) or Human Leukocyte Antigen (HLA) molecule. The interaction between antigenic peptides and HLA molecules is an essential element in the initiation and regulation of immune responses.

HLA molecules are polymorphic receptors expressed on the surfaces of a variety of cells in the body. The function of these receptors is to bind and display different peptide fragments on the surface of certain cells so the antigens can be recognized by T lymphocytes. This allows the immune system to survey the body for the presence of peptides derived from infectious agents or abnormal cancerous tissues. Such a peptide, when complexed with an HLA receptor, will trigger the T-cells to respond to the “foreign” agent.

Formation of a peptide-HLA complex and the subsequent T-cell recognition is highly sensitive to the peptide sequence. Thus, introduction of mutations into the activating wild type peptide can abrogate T-cell activation. Those organisms that present such mutations evade the host's immune response and therefore have a selective advantage.

It is believed that the diversity or polymorphism of HLA has been driven by co-evolving infectious disease threats. Many infectious agents have in turn co-evolved to escape the HLA-specific selective pressures of the host. This process of evolution and co-evolution is particularly evident in viruses like human immunodeficiency virus (HIV), herpes viruses and hepatitis viruses such as hepatitis C Virus (HCV).

For instance, the selection of HIV-1 variants that are associated with diminution or loss of CTL responses has been documented in various individuals with acute or late HIV-1 infection. However, other HIV-1 infected individuals have had a lack of demonstrable viral escape. To date, the frequency or importance of CTL escape mutation to global HIV evolution and pathogenicity in an HLA-diverse human population has not been fully elucidated. Moreover, there are many immune effects on HIV-1 sequences that are not well characterised.

For the aforementioned reasons current methods of DNA or protein analysis fail to account for many of the competing pressures that drive an animal's response to both a pathogenic microorganism and more specifically proteins produced by that microorganism.

The present invention seeks to provide methods to simultaneously define and analyse competing selective forces operating at the level of individual amino acids within protein from a pathogenic organism. Using such method(s) it is possible to analyse selective pressure exerted by individual polymorphic host genes on amino acids within particular microorganism protein sequence. It is also possible to examine the influence of a plurality of markers or a marker and other extrinsic variables on the variation of amino acids in a particular protein sequence. Gathering such data provides a means for monitoring, selecting and or individualisation of treatment or vaccination of a patient when infected with a particular microorganism or when perhaps they are in a high-risk group that is likely to be infected with a particular organism.

### SUMMARY OF THE INVENTION

The present invention provides methods of analysis, suitable for the identification and determination of bioactive amino acid sequences. It provides method(s) capable of determining the influence of variation in intrinsic host polypeptide or polynucleotide sequence(s) on the selection of particular amino acid sequences in microbial variants. It also provides methods for the analysis of the influence of variation in intrinsic host polypeptide in combination with one or more other variables such therapeutic agents (such as drugs or vaccines) on the selection of particular amino acid sequences in microbial variants. It provides methods for individualisation of a patient's treatment using such information as well as methods for determining patient susceptibility to treatment with a particular drug and offers the potential to tailor drug treatment regimes to individual patient. In a highly preferred form of the invention, a method is provided for identifying HLA allele-specific microorganism sequence polymorphisms that result from HLA restriction of antigen-specific cellular immune responses.

For ease of description of the present invention, HIV has been selected to illustrate how the methods described herein may be employed and how the data revealed from the methods may be used to prepare therapeutics suitable for treating HIV infected patients and patients at risk of HIV infection. It will be appreciated however that the methodologies so described may be applied to a wide range of analyses not least of which would include for example herpes virus infections and hepatitis (eg HCV) virus infections.

According to one embodiment, the invention provides a method for determining the influence of variation in host genes on selection of microorganisms with protein substitutions, comprising the steps of:
(a) Selecting a population of patients or animals infected with a particular microorganism and typing all individuals of the cohort for at least one selected intrinsic polymorphic marker involved in the host's response to the presence of the microorganism;
(b) Identifying and determining a portion of a polynucleotide sequence and or polypeptide sequence in the microorganism in a sufficient number of individuals from each type identified in step (a) in the cohort;
(c) Determining the consensus (i.e. most frequent) amino acid across the cohort at each residue position of the sequence analysed in step (b);
(d) Comparing the data obtained in step (a) and in step (b) to determine how the host polymorphic sequence(s) in step (a) increase or decrease the probability of a microorganism polymorphism at the first amino acid residue of interest in sequence determined in step (b); and
(e) Repeating step (d) for each amino acid identified in step (b) and comparing the data obtained.

According to a second embodiment the invention resides in a method for identifying the influence and interaction of variation in host polymorphic marker sequences and a second variable such as a therapeutic drug or vaccine on selection of microorganisms with particular amino acid variants, which method comprises the steps of:
a. selecting a population of patients or animals infected with a microorganism some of which have received the second variable as part of a treatment regime for the microorganism and typing the individuals of the cohort for at least one selected intrinsic host polymorphic marker sequence(s) involved in the host's response to the presence of the microorganism;
b. identifying and determining in a sufficient number of individuals from each type in the cohort part or all of a polynucleotide and or polypeptide sequence in the microorganism that is a potential or known target for the second variable, before and during exposure to the second variable and in similar but untreated individuals at a similar interval;
c. determine whether a change (“mutation”) has occurred at each residue of the sequence examined in step (b) between the time points identified in step (b);
d. comparing the data obtained in step (a) and the effect of presence or absence of exposure to the second variable in treated and untreated sequences and the data obtained in step (c) to determine how the polymorphic sequence(s) in step (a) and exposure to the second variable may affect the probability of mutation of the first amino acid residue of interest in step (c);
e. repeating step (d) for each amino acid in the sequence determined in step (c).

According to a further embodiment of the present invention there is provided a method to design therapeutics capable of inducing a specific T-cell response in a patient, that method comprising the steps as described above and then analysing the data to identify polymorphisms arising in a virus population as a result of infection of that population, which polymorphisms are HLA associated.

According to a further embodiment of the present invention there is provided a method to test the likely efficacy of a particular therapeutic in a particular population.

According to a further embodiment of the present invention there is provided a method to identify T cell epitopes, that method comprising the steps as described above and then analysing the data to identify the polymorphism frequency arising in a virus population as a result of infection of that population, which polymorphisms are HLA associated.

According to a further embodiment of the present invention there is provided a method to subclassify, prognosticate and monitor infectious diseases.

According to a further embodiment of the present invention there is provided a method to design a vaccine to prevent or delay the emergence of drug resistance in patients treated with a particular drug specific for a micro-organism, wherein the drug affects the replication of the microorganism at the nucleotide or amino acid level, which method comprises the steps of: performing the steps as described above and then analysing the data to identify the polymorphism frequency arising in a virus population in an infected individual which has been treated with an antiretroviral drug, wherein the polymorphism frequency is determined over the nucleotide or amino acid sequence regions where the drug is active in the micro-organism, and then designing one or more therapeutics which facilitate a T-cell response to cells that contain a virus population displaying one or more of the identified polymorphisms.

According to another aspect of the present invention there is provided a method of making an either an amino acid sequence designed according to the above methods or a vector construct capable of expressing that sequence in a patient, which is able to inducing a specific T-cell response in a patient infected with a micro-organism or at risk of infection with that microorganism.

Another aspect of the present invention is a method of preparing a composition comprising making either an amino acid sequence designed according to the above methods or a vector construct capable of expressing that sequence in a patient, which is able to inducing a specific T-cell response in a patient infected with a micro-organism or at risk of infection with that microorganism, and then combining the therapeutic with a pharmaceutically acceptable excipient.

The present invention also provides compositions for inducing a T-cell response to HIV in a mammal. The compositions comprising either an amino acid sequence designed according to the above methods or a vector construct capable of expressing that sequence in a patient, which is able to inducing a specific T-cell response in a patient infected with a microorganism or at risk of infection with that microorganism. Where the composition is used in the treatment of a patient it may also a pharmaceutically acceptable excipient. The immunogenic composition can further comprise a carrier, such as physiologic saline, and an adjuvant, such as incomplete freunds adjuvant, alum or montanide. Further the amino acid sequence may be modified as described herein to enhance its longevity or other desirable characteristics within an infected patient.

In other embodiments the present invention comprises methods for inducing a T lymphocyte response in a mammal against an antigen. The method comprises administering to the mammal either an amino acid sequence designed according to the above methods or a vector construct capable of expressing that sequence in a patient, which is able to inducing a specific T-cell response in a patient infected with a microorganism or at risk of infection with that microorganism.

In yet other embodiments the invention provides methods for treating or preventing a disease that is susceptible to treatment by a T cell response by administering a either an amino acid sequence designed according to the above methods or a vector construct capable of expressing that sequence in a patient, which is able to inducing a specific T-cell response in a patient infected with a micro-organism or at risk of infection with that microorganism.

Another aspect of the present invention is a method of invoking a cellular immune response in an animal by administering a composition comprising a pharmaceutically-acceptable excipient and an amino acid sequence adapted to contain a cellular immune response epitope comprising at least a viral polymorphism associated with a HLA allele type in a patient and an adjuvant. The cellular response may be a CD8+ T cell response, a CD4+ T cell, or both a CD8+ T cell and a CD4+ T cell response.

In an alternate form the present invention provides a method of invoking a cellular immune response in an animal by administering a composition comprising a pharmaceutically-acceptable excipient and an amino acid sequence adapted to contain at least a cellular immune response associated epitope that is highly conserved for a particular HLA type or a vector construct capable of expressing that amino acid sequence in an animal. The animal in which the immune response is invoked may be a mammal. In preferred embodiments the mammal may be a human, which may be either HIV positive or HIV negative.

Another aspect of the present invention is a method of delaying the onset of HIV in an animal exposed to infectious HIV by administering to the animal an inoculation of a pharmaceutically acceptable excipient and either an amino acid sequence designed according to the above methods or a vector construct capable of expressing that sequence in a patient, which is able to inducing a specific T-cell response in a patient infected with a micro-organism or at risk of infection with that micro-organism.

The present invention also provides an HIV amino acid sequences capable of inducing an HIV specific T-cell response in a patient infected with HIV or at risk of infection with HIV. Typically the T-cell response inducing amino acid sequence will be from seven to fifteen residues, and more usually from nine to eleven residues.

These and other aspects of the present invention are more fully described having regard to the following drawings and detailed description of the invention. The drawings and description are provided to aid in the description of the invention but should not be regarded as a limiting aspect of the invention.

### BRIEF DESCRIPTION OF THE DRAWINGS

The Figures are described as follows:

FIG. 1: Map of polymorphism rate at amino acid positions 95-202 of HIV-1 RT and known amino acid functional characteristics.

Map of amino acid positions 95-202 of HIV-1 RT showing the percentage of patients with change from population consensus amino acid at each position in pre-antiretroviral treatment HIV-1 RT sequences (n=185). Both conservative (grey bars) or non-conservative (solid black bars) amino acid substitutions are shown. The known functional characteristics of residues are marked as stability (S), functional (F), catalytic (C) and external (E) adjacent to the residue.

FIG. 2: Map of polymorphism rate at amino acid positions 20-227 of HIV-1 RT and associations with HLA-A and HLA-B alleles.

The known HLA-A and HLA-B restricted CTL epitopes (B. T. M. Korber et al., HIV Molecular Immunology Database 1999 (Theoretical Biology and Biophysics, New Mexico, 1999)) are marked as grey lines in Box A. Box D shows the percentage of patients with a different amino acid to that in the population consensus sequence at each position in most recent HIV-1 RT sequence (n=473). The HLA alleles that are significantly associated with polymorphism are shown above the polymorphic residue in Box B, along with the odds ratio (OR) for the association. The 15 HLA-specific polymorphisms within the 29 known CTL epitopes restricted to the same broad HLA allele are in grey text and the five at flanking residues are in black text. Clustered associations in black text may be within new or putative CTL epitopes. The boxed associations are those that remain significant after correction for total number of residues examined as described in the text. HLA-B*5101 is a subtype of HLA-B5, HLA-B44 is a subtype of HLA-B12 and HLA-A24 is a subtype of HLA-A9. In Box C, negative HLA associations are marked with ORs expressed as the inverse (1/OR), giving a value >1 for odds of not being different to consensus. These are also in grey or black text if within or flanking known CTL epitopes.

FIG. 3: HIV-RT amino acid sequences in all HLA-B5 patients.

The most recent amino acid sequence of HIV-1 RT in all 52 patients in the cohort with serologically defined HLA-B5 (patients 1-52), compared with population consensus sequence. HIV-1 RT sequences are grouped according to the HLA-B subtype of the patient. In all sequences, a dot (.) indicates no difference from consensus. Amino acids different to consensus are shown. Where quasispecies with different amino acids were detected, the most common amino acid is shown, except at position 135 where all detected amino acids in a mixed viral population are shown. All but one of the forty patients (98%) with the HLA-B*5101 subtype have a substitution of the consensus amino acid isoleucine (I) at position 135, most commonly with threonine (T). ¹The sequence without I135x is that of the single HLA-B*5101 patient who had HMRT during acute HIV infection. ²This patient did not have molecular genotyping. ³This patient was an HLA-B*5101/B*5201 heterozygote but was counted only once in the HLA-B*5101 group.

FIG. 4: Map of polymorphism rate at amino acid positions 1-90 of HIV-1 protease and associations with HLA-A and HLA-B alleles.

The known HLA-A and HLA-B restricted CTL epitopes are marked as grey lines in Box A. Box D shows the percentage of patients with a different amino acid to that in the population consensus sequence at each position in most recent HIV-1 protease sequence (n=493). The HLA alleles that are significantly associated with polymorphism are shown above the polymorphic residue in Box B, along with the odds ratio (OR) for the association. The boxed associations are those that remain significant after correction for total number of residues examined as described in the text. In Box C, negative HLA associations are marked with ORs expressed as the inverse (1/OR), giving a value >1 for odds of not being different to consensus.

FIG. 5(a) shows the relationship between the degree of viral adaptation to HLA-restricted responses and the HIV viral load.

FIG. 5(b) shows the frequency distribution of the number of beneficial residues in each six vaccine candidates (SIV, clade A virus, clade C virus, HXB2 virus, our population consensus virus, and our optimal vaccine) matched to each of the potential incoming infecting viruses in a West Australian population. The results indicate that ranking of vaccine candidate efficacy from highest to lowest in this population would be our optimised vaccine, our population consensus, the Clade B HXB2 virus, clade C virus, lade A virus and SIV.

FIG. 6 shows the frequency distribution of the estimated strength of HLA-restricted immune responses that would be induced by each of SIV, lade A virus, clade C virus, HXB2 virus, our population consensus virus sequence, and our optimal vaccine in response to each of the potential incoming viruses in a West Australian population using the viral load results as illustrated in the estimated change in viral load column shown in Table 6. The results indicate that ranking of vaccine candidate efficacy from highest to lowest in this population would be our optimised vaccine, our population consensus, lade C virus, lade A virus, the Clade B HXB2 virus and SIV.

FIG. 7 illustrates a putative HIV protease therapeutic

FIG. 8 illustrates a putative HIV RT therapeutic

### DETAILED DISCLOSURE OF THE INVENTION

General

Those skilled in the art will appreciate that the invention described herein is susceptible to variations and modifications other than those specifically described. It is to be understood that the invention includes all such variation and modifications. The invention also includes all of the steps, features, compositions and compounds referred to or indicated in the specification, individually or collectively and any and all combinations or any two or more of the steps or features.

The present invention is not to be limited in scope by the specific embodiments described herein, which are intended for the purpose of exemplification only. Functionally equivalent products, compositions and methods are clearly within the scope of the invention as described herein.

Sequence identity numbers (SEQ ID NO:) containing nucleotide and amino acid sequence information included in this specification are collected at the end of the description and have been prepared using the programme PatentIn Version 3.0. Each nucleotide or amino acid sequence is identified in the sequence listing by the numeric indicator <210> followed by the sequence identifier (e.g. <210>1, <210>2, etc.). The length, type of sequence and source organism for each nucleotide or amino acid sequence are indicated by information provided in the numeric indicator fields <211>, <212> and <213>, respectively. Nucleotide and amino acid sequences referred to in the specification are defined by the information provided in numeric indicator field <400> followed by the sequence identifier (e.g. <400>1, <400>2, etc.).

The entire disclosures of all publications (including patents, patent applications, journal articles, laboratory manuals, books, or other documents) cited herein are hereby incorporated by reference. No admission is made that any of the references constitute prior art or are part of the common general knowledge of those working in the field to which this invention relates.

As used herein the term “derived” and “derived from” shall be taken to indicate that a specific integer may be obtained from a particular source albeit not necessarily directly from that source.

Throughout this specification, unless the context requires otherwise, the word “comprise”, or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

### DESCRIPTION OF THE PREFERRED EMBODIMENT

The present invention provides methods of analysis, suitable for the identification and determination of bioactive amino acid sequences. It provides method(s) capable of determining the influence of variation in intrinsic host polypeptide or polynucleotide sequence(s) on the selection of particular amino acid sequences in microbial variants. It also provides methods for the analysis of the influence of variation in intrinsic host polypeptide in combination with one or more other variables such therapeutic agents (such as drugs or vaccines) on the selection of particular amino acid sequences in microbial variants. It provides methods for individualisation of a patient's treatment using such information as well as methods for determining patient susceptibility to treatment with a particular drug and offers the potential to tailor drug treatment regimes to individual patient. In a highly preferred form of the invention, a method is provided for identifying HLA allele-specific microorganism sequence polymorphisms that result from HLA restriction of antigen-specific cellular immune responses.

According to one embodiment, the invention provides a method for determining the influence of variation in host genes on selection of microorganisms with protein substitutions, comprising the steps of:
(a) Selecting a population of patients or animals infected with a particular microorganism and typing all individuals of the cohort for at least one selected intrinsic polymorphic marker involved in the host's response to the presence of the microorganism;
(b) Identifying and determining a portion of a polynucleotide sequence and or polypeptide sequence in the microorganism in a sufficient number of individuals from each type identified in step (a) in the cohort;
(c) Determining the consensus (i.e. most frequent) amino acid across the cohort at each residue position of the sequence analysed in step (b);
(d) Comparing the data obtained in step (a) and in step (b) to determine how the host polymorphic sequence(s) in step (a) increase or decrease the probability of a microorganism polymorphism at the first amino acid residue of interest in sequence determined in step (b); and
(e) Repeating step (d) for each amino acid identified in step (b) and comparing the data obtained.

Any univariate or multivariate statistical analysis may be employed in step (d) in the present invention. Preferably, the data obtained is analysed in a multiple variable logistic regression model. For example, the data obtained in step (a) may be employed as the explanatory co-variable and the data obtained in step (b) as the outcome (or response) variable in the model. Where such analysis is performed in this manner a polymorphism may be ascribed a value such as one (1) and no polymorphism may be ascribed an alternate value such as zero (0) as the outcome of interest.

Data from such an analysis will reveal regions of amino acid sequence that are prone to or resistant to variation. The amino acids prone to variation are likely to be involved in external biological interactions involving the analysed protein or they may represent regions of the protein sequence that may accommodate compensatory changes allowing variations in the sequence in other localities. Amino acid residues resistant to change are more likely to have critical structural, catalytic or functional properties. Using the associations between host and microorganisms polymorphisms, it is possible to identify putative regions of the microorganism's sequence that may have been selectively modified to evade influence of the host's immunological response. For example, the identified regions may represent HLA restricted CTL related epitopes, which the microorganism has selectively modified to evade a host CTL response. It should be appreciated that such regions may suggest amino acid sequences that may be valuable for therapeutic design. Alternatively, where a negative association is observed (ie. amino acids resistant to polymorphic variation in the presence of particular host gene polymorphisms) this may represent an amino acid residue that has been selected for by selective pressures to evade protective responses in previous hosts infected with the organism. Such amino acids may be highly significant as they may represent residues of the microorganisms that are appropriate targets for drugs or prophylactic or therapeutic vaccine therapy.

Preferably, the polymorphic sequence selected in step (a) is associated with an infected animals response to the microorganism that it is infected with. By “associated” is meant either directly or indirectly involved in the host's response to microorganism. In one particularly preferred form of the invention the intrinsic host polymorphic marker nucleic acid sequence(s) are those forming the HLA. For example, the HLA type marker may be HLA Class I (A, B, or C) or HLA Class II (DR, DQ). Alternatively, the marker nucleic acid sequence may be more specific to the microorganism in that it encodes a receptor or other protein actively engaged in host-microorganism interaction such as chemokine receptors like CCR5 involved in HIV binding.

Methods for determining intrinsic host marker types and or for identifying polymorphisms in the microorganism sequence will be generally known to those skilled in the field. Such methods may include, but are not limited to, direct DNA sequencing or analyses such as RFLP, SNP, SSO, SSP, variable number of tandem repeat (VNTR), etc. Given the relative ease with which sequencing may be now performed, preferably the sequences are directly sequences.

Methods described herein may be employed to examine selective pressures confronting a wide range of organisms that exhibit pathogenic traits in a host. Such organisms include but are not limited to bacteria, fungi, mycobacterium, viruses and virus-like particles. It should be appreciated that the methods described herein will have particular value in the examination of microorganisms that have adapted to evolve rapidly. Examples of such organisms include HIV and AIDS related viruses, herpes viruses and the hepatitis related viruses such as HCV and HBV.

Where the methods described herein refer to identifying and determining a portion of a polynucleotide and or polypeptide sequence one skilled in the art will understand that the sequences of either may be determined by any means known in the art. If only the polynucleotide sequence is known, the polypeptide sequence may be theoretically determined or directly sequenced as required.

It will be appreciated that the portion of the polynucleotide or polypeptide sequence that may be examined may be a short sequence of say only 20 or 30 amino acids or nucleotides extending to a much larger sequences encompassing a complete gene or protein sequence. Preferably, it will comprise a complete gene or protein sequence.

To effectively examine the influence of selective pressures exerted on a microorganism in the host, the host polymorphic gene sequence selected in step (a) should preferably be a sequence that is either directly or indirectly involved in the interaction between the host and the microorganism. Generally, for internal proteins of the microorganism therapeutic agents directly or indirectly interacting with those proteins or HLA genes are relevant. For proteins expressed on the external surface of the microorganism a wider array of other polymorphic host factors may also be relevant. For example, where the HIV reverse transcriptase (RT) gene (an internal protein of HIV) is being examined HIV reverse transcriptase inhibitor drugs and HLA alleles are most relevant. If, for example, the HIV envelope protein is examined, effects associated with chemokine receptor blockers or fusion inhibitor drugs, HLA alleles, anti-HIV antibody responses, CCR5 and CXCR4 genotype or any other polymorphic genes, encoding products targeting or interacting with envelope proteins may be considered.

To determine whether polymorphisms in the sequences selected in step (b) in the study cohort are distributed randomly or are associated with explanatory covariates as a result of selective pressure, the population consensus sequence is preferably used as a reference sequence and is determined by assigning the most common amino acid in the population at each position. Alternatively, and depending on the analysis being performed, the first sequence obtained in each individual host or a published reference sequence can be used as the reference sequence. Generally the outcome assessed is any change in the amino acid (even a low but detectable level of mutated or variant sequence) from the reference sequence of the microorganism being examined. Alternatively, the analysis may be refined to limit the examination of a specific or characteristic amino acid change at a particular residue (for example a change from M to V at position 184 of HIV reverse transcriptase protein).

The power of the presented method to detect the effect of host gene variants on microorganism polymorphism increases with improved resolution of the host genotyping and increasing amounts of data (the number of individuals with host genotyping and micro organism sequencing). The statistical power to detect the effect of any individual intrinsic polymorphic marker like an HLA allele in these models depended on the frequency of the allele in the population and the frequency of polymorphism at the amino acid position being examined. An initial power calculation may be performed for each position to determine for which alleles there is a reasonable power to detect an association if it existed (for example at least 30% power to detect an OR>2.0 or <0.5). The analysis can then be restricted to the identified alleles alone. This approach reduces the number of statistical comparisons being made and also identifies the allele/site combination for which there was insufficient power to detect an association even if one is present (such as might become apparent with a larger set of data).

If the frequency of the explanatory variable (i.e. the host polymorphisms) is low and the frequency of the outcome (i.e. the microorganism polymorphism) is also low then there will be less power to detect negative associations than to detect positive associations. For example, at a HLA allele frequency of 10.9 and an HIV polymorphism frequency of 4.0%, there is 30% power to detect an odds ratio of 2.0 (ie a positive association) but only 5.6% power to detect an equivalent negative odds ratio of 0.5.

Preferably, only those intrinsic polymorphic markers that have a degree of univariate association with polymorphism (for example with P≦0.1) are examined at each viral residue in subsequent analyses. Preferably, final covariates in the logistic regression models are capable of withstanding a standard forward selection and backwards elimination procedure. Permutation tests based on the logistic models may also be used to determine the exact P-values for associations (see, for example, F. L. Ramsey and D. W. Schafer in The Statistical Sleuth. A course in methods of data analysis, (Duxbury Press, 1997), chapter 2.

Analyses of large sets of genetic data such as these are hampered by statistical difficulties introduced by multiple statistical comparisons and large numbers of potential explanatory variables. These problems may be minimised using any or all of the following methods:
a. Restricting explanatory covariates examined to those with power to show an association;
b. Restricting explanatory covariates examined to those which show some level of association with the outcome (e.g. p>0.1) on univariate analysis;
c. Restricting explanatory covariates examined to those with an adequate number of outcomes (e.g. “mutation”>5);
d. Forward and then backward covariate selection process in the logistic regression model; and
e. Randomly assigning the host genotyping results to other individuals and then run the entire analysis and repeat the process a large number of times (“n”, e.g. 1000) to determine the number (“c”) of statistically significant associations (p<0.05) that may be expected by chance alone for each host allele at each micro organism residue. This information can be used to calculate P values corrected for multiple comparisons using the function 1−(1−P)^{20f} where f is equal to “c” divided by “n” and P is the p value uncorrected for multiple comparisons generated in steps (e).

The associations which remain significant (generally <0.05) after correction for multiple comparisons are more likely to be true associations. The odds ratio of the statistically significant association identified by the logistic regression model give a measure of the likely strength of the biological effect.

The results of all the individual models are desirably plotted together on a map of the amino acid sequence determined in step (c). Polymorphisms specific for a particular intrinsic polymorphic marker may be found to cluster along the sequence.

According to a second embodiment the invention resides in a method for identifying the influence and interaction of variation in host polymorphic marker sequences and a second variable such as a therapeutic drug or vaccine on selection of microorganisms with particular amino acid variants, which method comprises the steps of:
a. selecting a population of patients or animals infected with a microorganism some of which have received the second variable as part of a treatment regime for the microorganism and typing the individuals of the cohort for at least one selected intrinsic host polymorphic marker sequence(s) involved in the host's response to the presence of the microorganism;
b. identifying and determining in a sufficient number of individuals from each type in the cohort part or all of a polynucleotide and or polypeptide sequence in the microorganism that is a potential or known target for the second variable, before and during exposure to the second variable and in similar but untreated individuals at a similar interval;
c. determine whether a change (“mutation”) has occurred at each residue of the sequence examined in step (b) between the time points identified in step (b);
d. comparing the data obtained in step (a) and the effect of presence or absence of exposure to the second variable in treated and untreated sequences and the data obtained in step (c) to determine how the polymorphic sequence(s) in step (a) and exposure to the second variable may affect the probability of mutation of the first amino acid residue of interest in step (c);
e. repeating step (d) for each amino acid in the sequence determined in step (c).

While the intrinsic polymorphic marker may be the only covariate examined in the above method, it should be understood by those of ordinary skill that the defined methods also present a capacity to allow for an examination of other selective pressures that may serve as variables and which exert selective forces on microorganisms driving evolutionary change. Any variable capable of exerting a selective force on a microbial population in a patient may be examined by this method. For example the selective pressure might be the influence of a particular drug or therapeutic agent such as Zidovudine (or AZT) in the case of HIV infection. It may be the influence of a particular antibiotic in the case of a bacterial infection or the presence or absence of another microorganism in the case of a mixed population of organisms in a patient. Alternatively it might be a particular antibody or antibody population or a gene therapy system (eg. antisense related therapy).

Such analyses seek to examine competitive pressures between the host's intrinsic polymorphic marker and the second covariate on variation rates of the sequence selected in step (b). For example, where the host polymorphic marker is an HLA Allele, the microorganism is HIV-1, the sequence selected in step (b) is the reverse transcriptase gene (RT gene) and the selective pressure is cause by a therapeutic agent such as an antiretroviral drug, the HLA allele and antiretroviral drugs may exert competitive synergistic or antagonistic pressures at sites within the viral RT sequence.

By analysing the effects of the intrinsic marker and the therapeutic in the presented method it is possible to identify what influence the antiviral and or the HLA type may have on mutation or variation of DNA nucleotides or amino acid residues of the virus. One of ordinary skill in the field will understand such data, which provides a unique tool to individualise patient treatment regimes. Individualisation of antiretroviral therapy may conceivably be improved by using the methods described herein to identify synergistic or antagonistic interactions between immune pressure and drug pressures. Using this information it may be possible to identify whether the HLA restricted immune responses are exerting selective pressures synergistic or antagonistic to those being exerted by the therapeutic agent or agents. If it is, then the antiretroviral drug regime may be varied for members of the population with a particular HLA genotype and HIV sequence. Thus the method effectively provides a means for identifying the sensitivity or resistance of a particular type of patient to a particular drug regime.

According to preferred form of the second embodiment, the invention resides in a method for determining the influence and interaction of variation in host polymorphic marker sequences and therapeutic drugs on selection of microorganisms with particular amino acid variants, which method comprises the steps of:
(a) Selecting a population of patients or animals infected with a microorganism some of whom have received at least one pharmaceutical(s) intended for the treatment of the presence of the microorganism and typing the individuals of the cohort for at least one selected intrinsic host polymorphic marker sequence(s) involved in the host's response to the presence of the microorganism;
(b) Identifying and determining part or all of a polynucleotide or polypeptide sequence in the microorganism that is a potential target of the pharmaceutical in each treated individual of the cohort before and during exposure to the pharmaceutical and in similar but untreated individuals at a similar interval;
(c) Determining whether a change (“mutation”) has occurred at each residue of the sequence examined in step (b) between the time points identified in step (b);
(d) Comparing the data obtained in step (a) and the effect of presence or absence of exposure to the pharmaceutical between treated and untreated sequences and the data obtained in step (c) to determine how the polymorphic sequences in step (a) and pharmaceutical exposure may affect the mutation of the first amino acid residue of interest in step (c); and
(e) Repeat step (d) for each amino acid in the sequence determined in step (c).

As used herein the mutation relates to the change in an amino acid in an on treatment or post treatment sequence compared to a pre-treatment sequence in each individual. In an alternative form of the analysis the population consensus or a published reference sequence can be used as the reference sequence in which case mutation is defined as a change in an amino acid on treatment or post treatment in each individual compared to the population defined reference sequence.

Data from the above analyses will reveal the impact of competing pressures on the relative mutation of a particular amino acid or a group of amino acids in a sequence. Moreover, such analyses will provide a means to analyse individual interactive pressures on particular polymorphic changes in the microorganism sequence.

As in the previous embodiment any statistical method capable of either univariate or multivariate analysis may be employed in step (d). Preferably however the data is compared in a multivariable logistic regression model. For example, the data obtained in step (a) and from the presence or absence of exposure to the second variable between the two sequences may be used as separate explanatory covariates and the data obtained in step (c) may be used as the outcome variable in the model. Where such an analysis is conducted the outcome may be defined as one value (eg. zero) if the amino acid at the second time point is the same as that at the first time point and another value (eg. one) if the amino acid is different to that at the first time point. In addition or in an alternate form of analysis, the method may be used to examine the impact of HLA alleles on a characteristic anti-retroviral drug resistance change of one amino acid to another by assigning one value (one) to the change and another value (zero) where there is no change. For example, if an examination were conducted to determine the impact, if any, of HLA alleles on the characteristic lamivudine resistance mutation M184V, the presence of a change (V at position 184 of HIV reverse transcriptase) would be assigned one value such as 1 and the absence of a change would be assigned a second value such as 0. By comparing such data it is possible to identify the impact of the antiretroviral drugs and the HLA alleles on that amino acid change. Using such information it may be possible to define particular treatment regimes for patients of a specific HLA type.

Some amino acid changes require more than one (i.e. at least two or three) DNA nucleotide changes. Such amino acid changes suggest particularly strong selective pressure which may be relevant to drug or vaccine design or individualisation of treatment.

Polymorphisms or mutations at one residue of the microorganism may be linked or associated with polymorphism or mutation elsewhere in the microorganism. Changes at other residues in the microorganism can be included as explanatory covariates in the logistic model to identify possible compensatory or secondary polymorphisms or mutations. However, a compensatory mutation or mutations may act as intermediate outcomes and therefore their inclusion as explanatory covariates in a multivariate model may abrogate or hide the true primary explanatory influence of HLA alleles or drugs. Those skilled in the art will appreciate that inclusion of intermediate outcomes as explanatory covariates in the multivariate model may result erroneous interpretation of the findings by those less skilled in the art.

If different individuals in the cohort have been sequenced on a different number of occasions in step (b) then the logistic regression model can be modified using general estimating equation methodology to make the appropriate adjustments to prevent those individuals with more sequences contributing disproportionately to the model compared to individuals with fewer sequences.

In a highly preferred form the invention resides in a method comprising the steps of:
(a) HLA sequencing a large population of hosts infected with HIV;
(b) Sequencing the whole or part of the dominant HIV species in each patient;
(c) Defining the consensus sequence for HIV by determining the most common amino acid residue at each residue position of the virus;
(d) At each organism residue:
   (i) Determining for each individual (patient) whether the HIV amino acid residue of interest is the same (“non mutated) or different (“mutated”) compared to the consensus residue;
   (ii) Performing a multivariate (in this case logistic) regression model with mutated amino acids being assigned a value of (1) or non-mutated amino acids being assigned a value of (O) as the outcome of interest;
   (iii) Examining one or more of the following potential explanatory co-variates in the multivariate model looking for associations with the outcome of interest:
      (1) HLA allele of the individual patient;
      (2) Therapeutic drugs targeting the protein of interest taken by the host (e.g. reverse transcriptase inhibitor anti-retroviral drugs where HIV reverse transcriptase is being examined, protease inhibitors where HIV protease is being examined); and/or
      (3) Mutations at other positions in the host protein; and
   (iv) Interpret the findings.

Having regard to the nature of the methods described herein one of ordinary skill in the field will appreciate that the proposed method(s) of analysis will have wide application for examining protein relationships and for the analysis of bioactive molecules. Some of those uses are illustrated below:
1. To examine the Influence of putative Class I or II and escape or non-escape on either the dynamic equilibrium that determines the quantity of organism measured in the host (eg viral set point).
2. The influence of HLA type on risk of transmission in for example HIV discordant pairs (non-transmission), in HIV concordant pairs (transmission) or in any other type of infection.
3. The influence and interaction of HLA restricted immune pressure, codon usage and other polymorphisms in the organism on mutational pathway induced by therapy—eg whether an L90M or a D30N primary drug resistance mutation in HIV protease is induced by nelfinavir.
4. It provides methods for vaccine antigen selection.
5. It provides a method for examining external proteins (e.g. envelope proteins) for their interaction with HLA restricted immune pressure and or antibodies and or Chemokine receptor usage/switching and or escape from Chemokine receptor blockers or fusion inhibitors.
6. It also provides a method for examination of protein structure/function relationship.
7. It provides a method to individualise anti-microbial therapy. For example the method provides a means to select which of many possible different contemporary standard of care combinations of anti-retroviral therapy should be most effective for the treatment of an individual patient infected with HIV.

According to a further embodiment of the present invention there is provided a method to design therapeutics capable of inducing a specific T-cell response in a patient, that method comprising the steps as described above and then analysing the data to identify polymorphisms arising in a virus population as a result of infection of that population, which polymorphisms are HLA associated.

According to this method the individual is HLA typed and the genes encoding potential microbial protein targets (for example HIV reverse transcriptase and protease) are sequenced. The positive and negative associations between HLA alleles and microbial polymorphisms are determined in a large population of microbial infected individuals. Ideally the population should be the same or similar to the population from which the individual in question was drawn. The microbial amino acid residues that have known associations with the HLA alleles present in the individual in question are then examined.

From such analyses it will be possible to identify specific associations where the polymorphism frequency is such that a change in the amino acid or nucleotide is associated with a particular HLA type and is associates with T-cell escape. Preferable, the frequency of polymorphism selected for analysis is greater than 10%, more preferably greater than 15% and desirably greater than 20%, 25%, 30%, 35%, 40%, 45%, 50%, 55%, or 60%. Such data will reveal sequences of amino acids that potentially encode T-cell epitopes. Such data will also provides a sequence of amino acids that can also then be used in the development of a therapeutic. For example the therapeutic would be designed to encode the amino acid region in which the escape mutation exists, so as to prevent the escape mutation from having its effect. The examples provided herein illustrate how such sequences may be generated from the data obtained by the above method.

According to a further embodiment of the present invention there is provided a method to identify T cell epitopes, that method comprising the steps as described above and then analysing the data to identify the polymorphism frequency arising in a virus population as a result of infection of that population, which polymorphisms are HLA associated.

According to a further embodiment of the present invention there is provided a method to design a vaccine to prevent or delay the emergence of drug resistance in patients treated with a particular drug specific for a micro-organism, wherein the drug affects the replication of the microorganism at the nucleotide or amino acid level, which method comprises the steps of: performing the steps as described above and then analysing the data to identify the polymorphism frequency arising in a virus population in an infected individual which has been treated with an antiretroviral drug, wherein the polymorphism frequency is determined over the nucleotide or amino acid sequence regions where the drug is active in the micro-organism, and then designing one or more therapeutics which facilitate a T-cell response to cells that contain a virus population displaying one or more of the identified polymorphisms.

Where the method is employed to individualise anti-microbial therapy the individual is HLA typed and the genes encoding potential microbial protein targets of anti-microbial therapy (for example HIV reverse transcriptase and protease) are sequenced. The positive and negative associations between HLA alleles and microbial polymorphisms are determined in a large population of microbial infected individuals. Ideally the population should be the same or similar to the population from which the individual in question was drawn. The microbial amino acid residues that have known associations with the HLA alleles present in the individual in question are then examined. Anti-microbial drugs are then selected that: 1) favour the development of mutations at residues that have the population consensus at sites of negative HLA specific associations in the population and at residues that do not have the population consensus at the site of positive HLA specific associations in the population; or 2) resist the development of mutations at residues that have the population consensus at sites of positive HLA specific mutation in the population and at residues that do not have the population consensus at the site of negative HLA specific associations in the population. If more than one anti-microbial therapy is used, it is possible to combine agents that have competing effects at particular residues (i.e. a positive association in the population with one drug and a negative association with the second at the same residue) or proven in-vitro or in-vivo synergistic properties.

Methods to Design a Vaccine

The foregoing methodologies provide a means to identify polymorphic regions that may be used in the development of a therapeutic. Once those regions have been located a therapeutic vaccine is been preferably designed using the following principles:
1. Encode common resistance mutations
2. Encode putative “fitness mutations” where these do not interfere with common key mutations
3. Use whole protein as much as possible but avoid long stretches of wild-type amino acids as response to wild type sequence is relatively undesirable
4. Use the optimised consensus-like sequence described in Example 1 as the backbone (i.e. the amino acid sequence at the residues that are not sites of anti-retroviral resistance mutation). Where possible (e.g. protease) use a backbone known to fold appropriately (e.g. a real isolate) as antigen stability may be better.
5. Where resistance mutations are close together (<4 amino acids) generate separate fragments expressing only a single resistant epitope, as responses to epitopes containing 2 resistance mutations are relatively undesirable
6. For fragments containing a single mutation, encode 7 amino acids on either side to enhance development of CD8 T cell response to encoded mutation and reduce likelihood of response to wild-type sequence
7. However, encode as few as possible separate fragments as responses to amino acids sequences which overlap 2 fragments (irrelevant epitopes) is undesirable
8. Separate fragments which contain same coding sequence as much as possible as lessens potential for recombination during construction Method of making an amino acid sequence

According to another aspect of the present invention there is provided a method of making either an amino acid sequence designed according to the above methods.

A full length amino acid sequence of the instant invention can be prepared using well known recombinant DNA technology methods such as those set forth in Sambrook et al. (Molecular Cloning: A Laboratory Manual, Cold Spring Harbor Laboratory Press, Cold Spring Harbor, N.Y. [1989]) and/or Ausubel et al., eds, (Current Protocols in Molecular Biology, Green Publishers Inc. and Wiley and Sons, N.Y. [1994]).

A gene or cDNA encoding protein or fragment thereof may be obtained for example by PCR amplification of a micro-organism sequence. Improved methods of cloning in vitro amplified nucleic acids are described in Wallace et al., U.S. Pat. No. 5,426,039.

Alternatively, a gene encoding the polypeptide or fragment may be prepared by chemical synthesis using methods well known to the skilled artisan such as those described by Engels et al. (Angew. Chem. Intl. Ed., 28:716-734 [1989]). These methods include, inter alia, the phosphotriester, phosphoramidite, and H-phosphonate methods for nucleic acid synthesis. A preferred method for such chemical synthesis is polymer-supported synthesis using standard phosphoramidite chemistry. Typically, the DNA encoding the polypeptide will be several hundred nucleotides in length. Nucleic acids larger than about 100 nucleotides can be synthesized as several fragments using these methods. The fragments can then be ligated together to form the full length polypeptide. Usually, the DNA fragment encoding the amino terminus of the polypeptide will have an ATG, which encodes a methionine residue. This methionine may or may not be present on the mature form of the polypeptide, depending on whether the polypeptide produced in the host cell is secreted from that cell.

The gene or cDNA so isolated can be inserted into an appropriate expression vector for expression in a host cell. The vector is typically selected to be functional in the particular host cell employed (i.e., the vector is compatible with the host cell machinery such that amplification of the gene and/or expression of the gene can occur). The polypeptide or fragment thereof may be amplified/expressed in prokaryotic, yeast, insect (baculovirus systems) and/or eukaryotic host cells.

The amino acid sequences may then recovered and purified from the cell cultures by methods used heretofore, including ammonium sulfate or ethanol precipitation, acid extraction, anion or cation exchange chromatography, phosphocellulose chromatography, hydrophobic interaction chromatography, affinity chromatography, hydroxyapatite chromatography and lectin chromatography. It is preferred to have low concentrations (approximately 0.1-5 mM) of calcium ion present during purification (Price, et al., J. Biol. Chem., 244:917 (1969)). Protein refolding steps can be used, as necessary, in completing configuration of the mature protein. Finally, high performance liquid chromatography (HPLC) can be employed for final purification steps.

The amino acid sequences of the present invention may be a naturally purified product, or a product of chemical synthetic procedures, or produced by recombinant techniques from a prokaryotic or eukaryotic host (for example, by bacterial, yeast, higher plant, insect and mammalian cells in culture).

Method of Making a Vector Construct Capable of Expressing that Sequence in a Patient, which is Able to Inducing a Specific T-Cell Response

According to another aspect of the present invention there is provided a method of making a vector construct capable of expressing that sequence in a patient, which is able to inducing a specific T-cell response in a patient infected with a micro-organism or at risk of infection with that microorganism.

According to this method gene is isolated and then inserted into a vector construct capable of expressing that sequence in a patient, which is able to inducing a specific T-cell response in a patient.

For example, viral transduction methods may comprise the use of a recombinant DNA or an RNA virus comprising a nucleic acid sequence that drives expression of an amino acid sequence encoding a polymorphism to infect a target cell. A suitable DNA virus for use in the present invention includes but is not limited to an adenovirus (Ad), adeno-associated virus (MV), herpes virus, vaccinia virus or a polio virus. A suitable RNA virus for use in the present invention includes but is not limited to a retrovirus or Sindbis virus. It is to be understood by those skilled in the art that several such DNA and RNA viruses exist that may be suitable for use in the present invention.

Adenoviral vectors have proven especially useful for gene transfer into eukaryotic cells (Strafford-Perricaudet, L., and M. Perricaudet. 1991. Gene transfer into animals: the promise of adenovirus. p. 51-61, In: Human Gene Transfer, Eds, O. Cohen-Haguenauer and M. Boiron, Editions John Libbey Eurotext, France). Adenoviral vectors have been successfully utilized to study eukaryotic gene expression (Levrero, M., et al. 1991. Defective and nondefective adenovirus vectors for expressing foreign genes in vitro and in vivo. Gene 101: 195-202), vaccine development (Graham, F. L., and L. Prevec (1992) Adenovirus-based expression vectors and recombinant vaccines. In Vaccines: New Approaches to Immunological Problems, (Ellis, R. V. Ed.), pp. 363-390. Butterworth-heinemann, Boston), and in animal models (Stratford-Perricaudet, et al. 1992. Widespread long-term gene transfer to mouse skeletal muscles and heart. J. Clin. Invest. 90, 626-630; Rich, et al. 1993. Development and analysis of recombinant adenoviruses for gene therapy of cystic fibrosis. Human Gene Ther. 4, 461-476). The first trial of Ad-mediated gene therapy in human was the transfer of the cystic fibrosis transmembrane conductance regulator (CFTR) gene to lung (Crystal, et al. 1994. Nature Genetics 8, 42-51). Experimental routes for administrating recombinant Ad to different tissues in vivo have included intratracheal instillation (Rosenfeld, et al. 1992. In vivo transfer of the human cystic fibrosis transmembrane conductance regulator gene to the airway epithelium. Cell 68, 143-155) injection into muscle (Quantin, B., et al. 1992. Adenovirus as an expression vector in muscle cells in vivo. Proc. Natl. Acad. Sci. USA 89, 2581-2584), peripheral intravenous injection (Herz, J. and R. D. Gerard. 1993. Adenovirus-mediated transfer of low density lipoprotein receptor gene acutely accelerates cholesterol clearance in normal mice. Proc. Natl. Acad. Sci. USA 90, 2812-2816) and stereotactic inoculation to brain (Le Gal La Salle, et al. 1993. An adenovirus vector for gene transfer into neurons and glia in the brain. Science 259, 988-990). The adenoviral vector, then, is widely available to one skilled in the art and is suitable for use in the present invention.

Adeno-associated virus (AAV) has recently been introduced as a gene transfer system with potential applications in gene therapy. Wild-type AAV demonstrates high-level infectivity, broad host range and specificity in integrating into the host cell genome (Hermonat, P. L., and N. Muzyczka. 1984. Use of adeno-associated virus as a mammalian DNA cloning vector: transduction of neomycin resistance into mammalian tissue culture cells. Proc. Natl. Acad. Sci. USA 81: 6466-6470). Herpes simplex virus type-1 (HSV-1) is attractive as a vector system for use in the nervous system because of its neurotropic property (Geller, A. I., and H. J. Federoff. 1991. The use of HSV-1 vectors to introduce heterologous genes into neurons: implications for gene therapy. In: Human Gene Transfer, Eds, O. Cohen-Haguenauer and M. Boiron, pp. 63-73, Editions John Libbey Eurotext, France; Glorioso, et al. 1995. Herpes simplex virus as a gene-delivey vectors for the central nervous system. In: Viral Vectors-Gene therapy and neuroscience application, Eds, M. G. Kaplitt and A. D. Loewy, pp. 1-23. Academic Press, New York). Vaccinia virus, of the poxvirus family, has also been developed as an expression vector (Smith, G. L., and B. Moss. 1983. Infectious poxvirus vectors have capacity for at least 25,000 base pairs of foreign DNA. Gene 25: 21-28; Moss, B. 1992. Poxyviruses as eukaryotic expression vectors. Semin. Virol. 3: 277-283; Moss, B. 1992. Poxviruses as eukaryotic expression vectors. Semin. Virol. 3: 277-283). Each of the above-described vectors are widely available to one skilled in the art and would be suitable for use in the present invention.

Retroviral vectors are capable of infecting a large percentage of the target cells and integrating into the cell genome (Miller, A. D., and G. J. Rosman. 1989. Improved retroviral vectors for gene therapy and expression. Biotechniques 7: 980-990). Retroviruses were developed as gene transfer vectors relatively earlier than other viruses, and were first used successfully for gene marking and transducing the cDNA of adenosine deaminase (ADA) into human lymphocytes.

“Non-viral” delivery techniques that have been used or proposed for gene therapy include DNA-ligand complexes, adenovirus-ligand-DNA complexes, direct injection of DNA, CaPO.sub.4 precipitation, gene gun techniques, electroporation, and lipofection (Mulligan, R. C. 1993. The basic science of gene therapy. Science 260: 926-932). Any of these methods are widely available to one skilled in the art and would be suitable for use in the present invention. Other suitable methods are available to one skilled in the art, and it is to be understood that the present invention may be accomplished using any of the available methods of transfection. Several such methodologies have been utilized by those skilled in the art with varying success (Mulligan, R. C. 1993. The basic science of gene therapy. Science 260: 926-932). Lipofection may be accomplished by encapsulating an isolated DNA molecule within a liposomal particle and contacting the liposomal particle with the cell membrane of the target cell. Liposomes are self-assembling, colloidal particles in which a lipid bilayer, composed of amphiphilic molecules such as phosphatidyl serine or phosphatidyl choline, encapsulates a portion of the surrounding media such that the lipid bilayer surrounds a hydrophilic interior. Unilammellar or multilammellar liposomes can be constructed such that the interior contains a desired chemical, drug, or, as in the instant invention, an isolated DNA molecule.

Methods of Treatment

In other embodiments the present invention comprises methods for inducing a T lymphocyte response in a mammal against an antigen. The method comprises administering to the mammal either an amino acid sequence designed according to the above methods or a vector construct capable of expressing that sequence in a patient, which is able to inducing a specific T-cell response in a patient infected with a microorganism or at risk of infection with that microorganism.

In yet other embodiments the invention provides methods for treating or preventing a disease that is susceptible to treatment by a T cell response by administering a either an amino acid sequence designed according to the above methods or a vector construct capable of expressing that sequence in a patient, which is able to inducing a specific T-cell response in a patient infected with a micro-organism or at risk of infection with that microorganism.

Another aspect of the present invention is a method of invoking a cellular immune response in an animal by administering a composition comprising a pharmaceutically-acceptable excipient and an amino acid sequence adapted to contain a cellular immune response epitope comprising at least a viral polymorphism associated with a HLA allele type in a patient and an adjuvant. The cellular response may be a CD8+ T cell response, a CD4+ T cell, or both a CD8+ T cell and a CD4+ T cell response.

In an alternate form the present invention provides a method of invoking a cellular immune response in an animal by administering a composition comprising a pharmaceutically-acceptable excipient and an amino acid sequence adapted to contain at least a cellular immune response associated epitope that is highly conserved for a particular HLA type or a vector construct capable of expressing that amino acid sequence in an animal. The animal in which the immune response is invoked may be a mammal. In preferred embodiments the mammal may be a human, which may be either HIV positive or HIV negative.

Another aspect of the present invention is a method of delaying the onset of HIV in an animal exposed to infectious HIV by administering to the animal an inoculation of a pharmaceutically acceptable excipient and either an amino acid sequence designed according to the above methods or a vector construct capable of expressing that sequence in a patient, which is able to inducing a specific T-cell response in a patient infected with a micro-organism or at risk of infection with that micro-organism.

With respect to treatment or prevention of HIV infection in humans, selection of a T-cell inducing amino acid sequence(s) useful in the present invention can be as set forth herein. By selecting one or more amino acid sequences that induce T-cell response to a HIV antigen, a response is capable of being generated that is able to kill (or inhibit) cells which are infected by or otherwise express the native HIV antigens. With respect to treatment or prevention of HIV 1 and 2 in humans, one or more amino acid sequences that induce a T-cell response to a HIV 1 or 2 antigen may be selected. The HIV T-cell-inducing amino acid sequence will usually have at least four, sometimes six, often seven or more residues, or a majority of amino acids of that amino acid sequence that are identical or homologous when compared to the corresponding portion of the naturally occurring HIV sequence. For example, those amino acid sequences which are preferred for stimulating HIV T-cell responses include one or more of the amino acid sequences identified as SEQ ID NO 2 to 10, 11, 13, 15, 17, 19, 21, 23, 25, 27, 29, 31 or 33:

The T-cell inducing amino acid sequences employed in the compositions and methods of the present invention need not be identical to specific amino acid sequences disclosed in aforementioned disclosures, and can be selected by a variety of techniques, for example, according to certain motifs as described above.

In some instances it may be desirable to combine two or more amino acid sequences which contribute to stimulating specific T-cell responses in one or more patients or histocompatibility types. The amino acid sequences in the composition can be identical or different, and together they should provide equivalent or greater biological activity than the parent amino acid sequence(s). For example, using the methods described herein, two or more amino acid sequences may define different or overlapping T-cell epitopes from a particular region, which amino acid sequences can be combined in a “cocktail” to provide enhanced immunogenicity of T-cell responses, and amino acid sequences can be combined with amino acid sequences having different MHC restriction elements. This composition can be used to effectively broaden the immunological coverage provided by therapeutic, vaccine or diagnostic methods and compositions of the invention among a diverse population. P In some embodiments the T-cell inducing amino acid sequences of the invention linked by a spacer molecule, or the T-cell amino acid sequences may be linked without a spacer. When present, the spacer is typically comprised of relatively small, neutral molecules, such as amino acids or amino acid mimetics, which are substantially uncharged under physiological conditions and may have linear or branched side chains. The spacers are typically selected from, e.g., Ala, Gly, or other neutral spacers of nonpolar amino acids or neutral polar amino acids. In certain preferred embodiments herein the neutral spacer is Ala. It will be understood that the optionally present spacer need not be comprised of the same residues and thus may be a hetero- or homo-oligomer. Preferred exemplary spacers are homo-oligomers of Ala. When present, the spacer will usually be at least one or two residues, more usually three to six residues.

The amino acid sequences of the invention can be combined via linkage to form polymers (multimers), or can be formulated in a composition without linkage, as an admixture. Where the same amino acid sequence is linked to itself, thereby forming a homopolymer, a plurality of repeating epitopic units are presented. When the amino acid sequences differ, e.g., a cocktail representing different antigen strains or subtypes, different epitopes within a subtype, different histocompatibility restriction specificities, or amino acid sequences which contain epitopes, heteropolymers with repeating units are provided. In addition to covalent linkages, noncovalent linkages capable of forming intermolecular and intrastructural bonds are also contemplated.

The amino acid sequences of the present invention and pharmaceutical and vaccine compositions thereof are useful for administration to mammals, particularly humans, to treat and/or prevent viral, bacterial, and parasitic infections. As the amino acid sequences are used to stimulate cytotoxic T-lymphocyte responses to infected cells, the compositions can be used to treat or prevent acute and/or chronic infection.

For pharmaceutical compositions, the T-cell amino acid sequences of the invention as described above will be administered to a mammal already suffering from or susceptible to the disease being treated. Those in the incubation phase or the acute phase of disease such as a viral infection, can be treated with the immunogenic amino acid sequences separately or in conjunction with other treatments, as appropriate. In therapeutic applications, compositions are administered to a patient in an amount sufficient to elicit an effective T-cell response to the disease and to at least partially arrest its symptoms and/or complications. An amount adequate to accomplish this is defined as “therapeutically effective dose.” Amounts effective for this use will depend on, e.g., the amino acid sequence composition, the manner of administration, the stage and severity of the disease being treated, the weight and general state of health of the patient, and the judgment of the prescribing physician, but generally range for the initial immunization (that is for therapeutic or prophylactic administration) from about 1.0 μg to about 50 mg, preferably 1 μg to 500 μg, most preferably 1 μg to 250 μg followed by boosting dosages of from about 1.0 μg to 50 mg, preferably 1 μg to 500 μg, and more preferably 1 μg to about 250 μg of amino acid sequence pursuant to a boosting regimen over weeks to months depending upon the patient's response and condition by measuring specific T-cell activity in the patient's blood. It must be kept in mind that the amino acid sequences and compositions of the present invention may generally be employed in serious disease states, that is, life-threatening or potentially life threatening situations. In such cases, in view of the minimization of extraneous substances and the relative nontoxic nature of the amino acid sequences, it is possible and may be felt desirable by the treating physician to administer substantial excesses of these amino acid sequence compositions.

Single or multiple administrations of the compositions can be carried out with dose levels and pattern being selected by the treating physician. In any event, the pharmaceutical formulations should provide a quantity of cytotoxic T-lymphocyte stimulatory amino acid sequences of the invention sufficient to effectively treat the patient.

For therapeutic use, administration should begin at the first sign of disease (e.g., HIV infection), to be followed by boosting doses until at least symptoms are substantially abated and for a period thereafter. In cases of established or chronic disease, such as chronic HIV infection, loading doses followed by boosting doses may be required. The elicitation of an effective T-cell response during early treatment of an acute disease stage will minimize the possibility of subsequent development of chronic disease such HIV carrier stage.

Treatment of an infected mammal with the compositions of the invention may hasten resolution of the disease in acutely afflicted mammals. For those mammals susceptible (or predisposed) to developing chronic disease the compositions of the present invention are particularly useful in methods for preventing the evolution of the disease. Where the susceptible individuals are identified prior to or during infection, for instance, as described herein, the composition can be targeted to them, minimizing need for administration to a larger population.

The amino acid sequence compositions can also be used for the treatment of established disease and to stimulate the immune system to eliminate virus-infected cells. Those with established disease can be identified as testing positive for virus from about 3-6 months after infection. As individuals may develop HIV infection because of an inadequate (or absent) T-cell response during the early phase of their infection, it is important to provide an amount of immuno-potentiating amino acid sequence compositions of the invention in a formulation and mode of administration sufficient to effectively stimulate a T-cell response. Thus, for treatment of established disease, a representative dose is in the range of about 1.0 μg to about 50 mg, preferably 1 μg to 500 μg, most preferably 1 μg to 250 μg followed by boosting dosages of from about 1.0 μg to 50 mg, preferably 1 μg to 500 μg, and more preferably 1 μg to about 250 μg per dose. Administration should continue until at least clinical symptoms or laboratory indicators indicate that the HIV infection has been substantially abated and for a period thereafter. Immunizing doses followed by boosting doses at established intervals, e.g., from one to four weeks, may be required, possibly for a prolonged period of time, as necessary to resolve the infection.

The pharmaceutical compositions for therapeutic treatment are intended for parenteral, topical, oral or local administration. Preferably, the pharmaceutical compositions are administered parenterally, e.g., intravenously, subcutaneously, intradermally, or intramuscularly. Thus, the invention provides compositions for parenteral administration that comprise a solution of the T-cell stimulatory amino acid sequences dissolved or suspended in an acceptable carrier, preferably an aqueous carrier. A variety of aqueous carriers may be used, e.g., water, buffered water, 0.4% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, methanol, and dissolving agents such as DMSO, etc.

The concentration of T-cell stimulatory amino acid sequences of the invention in the pharmaceutical formulations can vary widely, i.e., from less than about 1%, usually at or at least about 10% to as much as 20 to 50% or more by weight, and will be selected primarily by fluid volumes, viscosities, etc., in accordance with the particular mode of administration selected.

Thus, a typical pharmaceutical composition for intravenous infusion could be made up to contain 250 ml of sterile Ringer's solution, and 50 mg of amino acid sequence. Actual methods for preparing parenterally administrable compounds will be known or apparent to those skilled in the art and are described in more detail in for example, Remington's Pharmaceutical Sciences, 17th ed., Mack Publishing Company, Easton, Pa. (1985), which is incorporated herein by reference.

The amino acid sequences of the invention may also be administered via liposomes, which serve to target the amino acid sequences to a particular tissue, such as lymphoid tissue, or targeted selectively to infected cells, as well as increase the half-life of the amino acid sequence composition. Liposomes include emulsions, foams, micelles, insoluble monolayers, liquid crystals, phospholipid dispersions, lamellar layers and the like. In these preparations the amino acid sequence to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired amino acid sequence of the invention can be directed to the site of lymphoid cells, where the liposomes then deliver the selected therapeutic/immunogenic amino acid sequence compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size and stability of the liposomes in the blood stream. A variety of methods are available for preparing liposomes, as described in, e.g., Szoka et al., Ann. Rev. Biophys. Bioeng. 9:467 (1980), U.S. Pat. Nos. 4,235,871, 4,501,728, 4,837,028, and 5,019,369, incorporated herein by reference. For targeting to the immune cells, a ligand to be incorporated into the liposome can include, e.g., antibodies or fragments thereof specific for cell surface determinants of the desired immune system cells. A liposome suspension containing a amino acid sequence may be administered intravenously, locally, topically, etc. in a dose which varies according to, inter alia, the manner of administration, the amino acid sequence being delivered, and the stage of the disease being treated.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more amino acid sequence compositions of the invention, and more preferably at a concentration of 25%-75%.

For aerosol administration, the T-cell stimulatory amino acid sequence compositions are preferably supplied in finely divided form along with a surfactant and propellant. Typical percentages of amino acid sequences are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included, as desired, as with, e.g., lecithin for intranasal delivery.

In another aspect the present invention is directed to therapeutic that contain as an active ingredient an immunogenically effective amount of a composition of T-cell stimulating amino acid sequences as described herein. The amino acid sequence(s) may be introduced into a mammalian host, including humans, linked to its own carrier or as a homopolymer or heteropolymer of active amino acid sequence units. Such a polymer has the advantage of increased immunological reaction and, where different amino acid sequences are used to make up the polymer, the additional ability to induce antibodies and/or cytotoxic T cells that react with different antigenic determinants of the virus. Useful carriers are well known in the art, and include, e.g., thyroglobulin, albumins such as human serum albumin, tetanus toxoid, polyamino acids such as poly(D-lysine:D-glutamic acid), influenza protein and the like. The therapeutic can also contain a physiologically tolerable (acceptable) diluent such as water, phosphate buffered saline, or saline, and further typically include an adjuvant. Adjuvants such as incomplete Freund's adjuvant, aluminum phosphate, aluminum hydroxide, alum, or MONTANIDE® (Seppic, Paris, France; oil-based adjuvant with mannide oleate) are materials well known in the art. Upon immunization with a amino acid sequence composition as described herein, via injection, aerosol, oral, transdermal or other route, the immune system of the host responds to the therapeutic by producing large amounts of T-cell specific for the disease associated antigen, and the host becomes at least partially immune to the disease, or resistant to developing disease.

Therapeutic compositions containing the amino acid sequences of the invention are administered to a patient susceptible to or otherwise at risk of disease, e.g., viral infection, to enhance the patient's own immune response capabilities. Such an amount is defined to be a “immunogenically effective dose.” In this use, the precise amounts depend on the patient's state of health, age, the mode of administration, the nature of the formulation, etc. The amino acid sequences are administered to individuals of an appropriate HLA type, e.g., for therapeutic compositions of the following amino acid sequences, these will be administered to the identified HLA typed individuals.

| | |
|---|---|
| (i) | FLDGIDKAQEEHEKYHSNWRAM and HLA-B*4402 |
| (ii) | GKWSKSSMVGWPAVRERMRRAEP and HLA-C*0701 |
| (iii) | AQEEEEVGFPVRPQVPLRPMTYK and HLA-B*0702 |
| (iv) | SFRFGEETTTPSQKQEPIDKENY and HLA-B*4402 |
| (v) | RIGCQHSRIGIIRQRRARNGASR and HLA-DRBI-0701 |
| (vi) | KTIHTDNGSNFTSTTVKAACWWA and HLA-C*0501 |
| (vii) | TGADDTVLEEMNLPGRWKPKMIG and HLA-DRB1-1302 |
| (viii) | GEETTTPSQKQEPIDKENYPLAS and HLA-A*2402 |
| (ix) | WPVKTIHTDNGSNFTSTTVKAAC and HLA-B*4402 |
| (x) | MQRGNFRNQRKTVKCFNCGK and HLA-B*1801 |

A number of different animal model systems for HIV infection have been employed (Kindt et al., 1992). Non-human primates such as chimpanzees and pig-tailed macaques can be infected by HIV-1. Although CD4+ cells are not depleted in these systems, the animals are detectably infected by the virus and are useful in determining the efficacy of HIV therapeutics. Small animal models include chimeric models that involve the transplantation of human tissue into immunodeficient mice. One such system is the hu-PBL-SCID mouse developed by Mosier et al. (1988). Another is the SCID-hu mouse developed by McCune et al. (1988). Of the two mouse models, the SCID-hu mouse is typically preferred because HIV infection in these animals is more similar to that in humans. SCID-hu mice implanted with human intestine have been shown to be an in vivo model of mucosal transmission of HIV (Gibbons et al., 1997). Methods of constructing mammals with human immune systems are described in U.S. Pat. Nos. 5,652,373, 5,698,767, and 5,709,843.

The animals will be inoculated with a therapeutic of the present invention and later challenged with a dose of infectious virus. Efficacy of the therapeutic may be determined by methods known by those of skill in the art. Generally, a variety of parameters associated with HIV infection may be tested and a comparison may be made between vaccinated and non-vaccinated animals. Such parameters include viremia, detection of integrated HIV in blood cells, loss of CD4+ cells, production of HIV particles by PBMC, etc. The therapeutic will be considered effective if there is a significant reduction of signs of HIV infection in the vaccinated versus the non-vaccinated groups.

Of course, the inventor contemplates the application of the present invention as a therapeutic to HIV in humans. The inventors contemplate that testing of the present invention, as a therapeutic in humans will follow standard techniques and guidelines known by those of skill in the art. One important aspect of human application is the production of an effective immune response to the therapeutic. Although various ex vivo tests may be performed, such as measuring anti-HIV cellular responses, the ultimate test is the ability of the therapeutic to at least ameliorate infection by HIV or to significantly prolong the onset of AIDS in individuals receiving the therapeutic. The monitoring of the efficacy of HIV therapeutics in humans is well known to those of skill in the art and the inventor does not contemplate that the present invention would require the development of new methods of testing the efficacy of an HIV therapeutic.

The amino acid sequences may also find use as diagnostic reagents. For example, an amino acid sequence of the invention may be used to determine the susceptibility of a particular individual to a treatment regimen which employs the amino acid sequence or related amino acid sequences, and thus may be helpful in modifying an existing treatment protocol or in determining a prognosis for an affected individual. In addition, the amino acid sequences may also be used to predict which individuals will be at substantially protected from developing HIV infection.

Diagnostic Methods

According to a further embodiment of the present invention there is provided a method to subclassify, prognosticate and monitor infectious diseases.

Diagnostic and prognostic methods will generally be conducted using a biological sample obtained from a patient, which contains the microorganism. A “sample” refers to a sample of tissue or fluid suspected of containing the microorganism or a portion (eg amino acid sequence or nucleotide sequence) from an individual including, but not limited to, e.g., plasma, serum, spinal fluid, lymph fluid, the and samples of in vitro cell culture constituents.

According to the diagnostic and prognostic methods of the present invention, alteration of the amino acid sequence of the microorganism may be detected using anyone of the methods described herein. In addition, the diagnostic and prognostic methods can be performed to detect the frequency or rate of change of the amino acid sequence of the microorganism.

As used herein, the terms “diagnosis” or “prognosis,” as used in the context of the invention, are used to indicate 1) the classification of microorganisms displaying escape mutations, 2) the determination of the severity of the escape mutations, or 3) the monitoring of the disease progression, prior to, during and after treatment.

To detect the alteration of a wild-type microorganism nucleotide or amino acid sequence in a tissue, it is helpful to isolate the microorganism from a patient. Means for enriching microorganism preparations are known in the art and will depend on the type of organism being isolated.

A rapid preliminary analysis to detect polymorphisms in DNA sequences can be performed by looking at a series of Southern or northern blots of nucleotide material cut with one or more restriction enzymes, preferably with a large number of restriction enzymes. Northern or Southern blots displaying hybridising fragments indicate a possible mutation. If restriction enzymes that produce very large restriction fragments are used, then pulsed field gel electrophoresis (PFGE) may also be employed.

Detection of point mutations may also be accomplished by molecular cloning of the microorganism sequence and sequencing the allele(s) using techniques well known in the art. Alternatively, the gene sequences can be amplified directly from a nucleotide sequence preparation, using known techniques.

Some other useful diagnostic techniques for detecting the presence of polymorphisms to the gene include, but are not limited to: 1) allele-specific PCR; 2) single stranded conformation analysis (SSCA); 3) denaturing gradient gel electrophoresis (DGGE); 4) RNase protection assays; 5) the use of proteins which recognize nucleotide mismatches, such as the E. coli mutS protein; 6) allele-specific oligonucleotides (ASOs); and 7) fluorescent in situ hybridisation (FISH).

Alteration of mutated microorganism genes can also be detected by screening for alteration of a wild-type microorganism protein. Such alterations can be determined by amino acid sequence analysis in accordance with conventional techniques. More preferably, antibodies (polyclonal or monoclonal) may be used to detect differences in, or the absence of mutated microorganism proteins or peptides. The antibodies may be prepared as discussed below.

Antibodies specific for products of mutant alleles can be used to detect mutant microorganism amino acid sequence. Such immunological assays can be done in any convenient format known in the art. These include Western blots, immunohistochemical assays and ELISA assays. Any means for detecting an altered amino acid sequences can be used to detect alteration of a wild-type amino acid sequence.

In a preferred embodiment of the invention, antibodies will immunoprecipitate mutated amino acid sequence from solution as well as react with mutated amino acid sequences on Western or immunoblots of polyacrylamide gels.

Preferred embodiments relating to methods for detecting mutated amino acid sequences include enzyme linked immunosorbent assays (ELISA), radioimmunoassays (RIA), immunoradiometric assays (IRMA) and immunoenzymatic assays (IEMA), including sandwich assays using monoclonal and/or polyclonal antibodies.

Antibody Preparation Methods

An antibody of the present invention is typically produced by immunizing a mammal with an inoculum containing an amino acid sequences of this invention and thereby induce in the mammal antibody molecules having immunospecificity for immunizing amino acid sequence. The antibody molecules are then collected from the mammal and isolated to the extent desired by well known techniques such as, for example, by using DEAE Sephadex or Protein G to obtain the IgG fraction.

Exemplary antibody molecules for use in the diagnostic methods and systems of the present invention are intact immunoglobulin molecules, substantially intact immunoglobulin molecules and those portions of an immunoglobulin molecule that contain the paratope, including those portions known in the art as Fab, Fab′, F(ab′)₂ and F(v). Fab and F(ab′)₂ portions of antibodies are prepared by the proteolytic reaction of papain and pepsin, respectively, on substantially intact antibodies by methods that are well known. See for example, U.S. Pat. No. 4,342,566. Fab′ antibody portions are also well known and are produced from F(ab′)₂ portions followed by reduction of the disulfide bonds linking the two heavy reduction of the disulfide bonds linking the two heavy chain portions as with mercaptoethanol, and followed by alkylation of the resulting protein mercaptan with a reagent such as iodoacetamide. An antibody containing intact antibody molecules are preferred, and are utilized as illustrative herein.

The preparation of antibodies against a polymorphism containing amino acid sequence is well known in the art. See Staudt et al., J. Exp. Med., 157:687-704 (1983), or the teachings of Sutcliffe, J. G., as described in U.S. Pat. No. 4,900,811, the teaching of which are hereby incorporated by reference. Briefly, to produce a polymorphism containing amino acid sequence antibody composition of this invention, a laboratory mammal is inoculated with an immunologically effective amount of a polymorphism containing amino acid sequence of this invention typically as present in a vaccine of the present invention. The anti-amino acid sequence antibody molecules thereby induced are then collected from the mammal and those immunospecific for both the polymorphism containing amino acid sequence are isolated to the extent desired by well known techniques such as, for example, by immunoaffinity chromatography.

To enhance the specificity of the antibody, the antibodies are preferably purified by immunoaffinity chromatography using solid phase-affixed immunizing polypeptide. The antibody is contacted with the solid phase-affixed immunizing polypeptide for a period of time sufficient for the polypeptide to immunoreact with the antibody molecules to form a solid phase-affixed immunocomplex. The bound antibodies are separated from the complex by standard techniques.

For amino acid sequences that contain fewer than about 35 amino acid residues, it is preferable to use the peptide bound to a carrier for the purpose of inducing the production of antibodies. One or more additional amino acid residues can be added to the amino- or carboxy-termini of the polypeptide to assist in binding the polypeptide to a carrier. Cysteine residues added at the amino- or carboxy-termini of the polypeptide have been found to be particularly useful for forming conjugates via disulfide bonds. However, other methods well known in the art for preparing conjugates can also be used. The techniques of polypeptide conjugation or coupling through activated functional groups presently known in the art are particularly applicable. See, for example, Aurameas, et al., Scand. J. Immunol., Vol. 8, Suppl. 7:7-23 (1978) and U.S. Pat. Nos. 4,493,795, 3,791,932 and 3,839,153. In addition, a site-directed coupling reaction can be carried out so that any loss of activity due to polypeptide orientation after coupling can be minimized. See, for example, Rodwell et al., Biotech., 3:889-894 (1985), and U.S. Pat. No. 4,671,958. Exemplary additional linking procedures include the use of Michael addition reaction products, di-aldehydes such as glutaraldehyde, Klipstein, et al., J. Infect. Dis., 147:318-326 (1983) and the like, or the use of carbodiimide technology as in the use of a water-soluble carbodiimide to form amide links to the carrier. Alternatively, the heterobifunctional cross-linker SPDP (N-succinimidyl-3-(2-pyridyidithio) proprionate)) can be used to conjugate peptides, in which a carboxy-terminal cysteine has been introduced.

Useful carriers are well known in the art, and are generally proteins themselves. Exemplary of such carriers are keyhole limpet hemocyanin (KLH), edestin, thyroglobulin, albumins such as bovine serum albumin (BSA) or human serum albumin (HSA), red blood cells such as sheep erythrocytes (SRBC), tetanus toxoid, cholera toxoid as well as polyamino acids such as poly D-lysine:D-glutamic acid, and the like. The choice of carrier is more dependent upon the ultimate use of the inoculum and is based upon criteria not particularly involved in the present invention. For example, a carrier that does not generate an untoward reaction in the particular animal to be inoculated should be selected.

The present inoculum contains an effective, immunogenic amount of an amino acid sequence as described herein, typically as a conjugate linked to a carrier. The effective amount of an amino acid sequence as described herein per unit dose sufficient to induce an immune response to the immunizing polypeptide depends, among other things, on the species of animal inoculated, the body weight of the animal and the chosen inoculation regimen is well known in the art. Inocula typically contain amino acid sequence concentrations of about 10 micrograms to about 500 milligrams per inoculation (dose), preferably about 50 micrograms to about 50 milligrams per dose. The term “unit dose” as it pertains to the inocula refers to physically discrete units suitable as unitary dosages for animals, each unit containing a predetermined quantity of active material calculated to produce the desired immunogenic effect in association with the required diluent; i.e., carrier, or vehicle. The specifications for the novel unit dose of an inoculum of this invention are dictated by and are directly dependent on (a) the unique characteristics of the active material and the particular immunologic effect to be achieved, and (b) the limitations inherent in the art of compounding such active material for immunologic use in animals, as disclosed in detail herein, these being features of the present invention.

Inocula are typically prepared from the dried solid amino acid sequence-conjugate by dispersing the amino acid sequence-conjugate in a physiologically tolerable (acceptable) diluent such as water, saline or phosphate-buffered saline to form an aqueous composition. Inocula can also include an adjuvant as part of the diluent. Adjuvants such as complete Freund's adjuvant (CFA), incomplete Freund's adjuvant (IFA) and alum are materials well known in the art, and are available commercially from several sources.

The antibody so produced can be used, inter alia, in the diagnostic methods and systems of the present invention to detect an amino acid sequence of the present invention in a body fluid sample. A typical example of such an antibody would be a monoclonal antibody.

A monoclonal antibody is typically composed of antibodies produced by clones of a single cell called a hybridoma that secretes (produces) only one kind of antibody molecule. The hybridoma cell is formed by fusing an antibody-producing cell and a myeloma or other self-perpetuating cell line. The preparation of such antibodies was first described by Kohler and Milstein, Nature, 256:495497 (1975), the description of which is incorporated by reference. The hybridoma supernates so prepared can be screened for the presence of antibody molecules that immunoreact with a polymorphism containing amino acid sequence.

Kits

The present invention contemplates a kit comprising specific probes for detection of an amino acid sequence that contains a polymorphism of interest where such a probe can be functionalised antibody protein, polyclonal antibody, monoclonal antibody, or antigen binding fragment of such proteins. Preferably, amino acid sequence is substantially identical to a sequence selected from SEQ ID NOS. 1-33.

### BEST MODE(S) FOR CARRYING OUT THE INVENTION

Further features of the present invention are more fully described in the following non-limiting Examples. It is to be understood, however, that this detailed description is included solely for the purposes of exemplifying the present invention. It should not be understood in any way as a restriction on the broad description of the invention as set out above.

Methods of molecular biology that are not explicitly described in the following examples are reported in the literature and are known by those skilled in the art. General texts that described conventional molecular biology, microbiology, and recombinant DNA techniques within the skill of the art, included, for example: Sambrook et al., Molecular Cloning: A Laboratory Manual, Second Edition, Cold Spring Harbor Laboratory Press, Cold Spring Harbor, N.Y. (1989); Glover ed., DNA Cloning: A Practical Approach, Volumes I and II, MRL Press, Ltd., Oxford, U.K. (1985); and Ausubel, F., Brent, R., Kingston, R. E., Moore, D. D., Seidman, J. G., Smith, J. A., Struhl, K. Current protocols in molecular biology. Greene Publishing Associates/Wiley Intersciences, New York.

### EXAMPLE 1

An Examination of HIV-1 Reverse Transcriptase (RT)

The following Examples illustrate the invention in the context of an examination of HIV-1 Reverse Transcriptase (RT). HIV-1 reverse transcriptase (RT) is highly expressed in virions and immunogenic in the early response to HIV-1. It will be appreciated by those skilled in the field that HIV-1 RT may be substituted for another suitable HIV protein or the sequences selected for examination may be derived from another virus or organism.

Data collection: Relationships between HIV-1 RT sequences in 473 participants of a Western Australian (WA) HIV Cohort Study and their HLA-A, -B and -DRB1 genotypes were examined. The HLA-A and -B alleles present in individuals included A1, A2, A3, A9, A10, A11, A19, A28, A31, A36, B5, B7, B8, B12, B13, B14, B15, B16, B17, B18, B21, B22, B27, B35, B37, B40, B41, B42, B55, B56, B58, B60 and B61.

The vast majority of patients in the cohort reside in or near the capital of Western Australia, Perth, which is one of the most geographically isolated cities in the world. New HIV-1 infections are most frequently acquired from sources within Western Australia (53.3%) or other states in Australia (24.3%), and less commonly from Asia (8.2%), Africa (5.1%), Europe (4.9%), North America (3.4%) or South America (0.8%). Participants have certain demographic, clinical and laboratory data collected routinely, including HLA class I serological typing and HLA class II sequence based typing. HIV-1 RT proviral DNA sequencing is performed at first presentation (prior to any antiretroviral treatment in 185 cases) and serially whilst on RT inhibitor therapy. This study encompasses data collected over approximately 2210 patient-years of observation.

The WA Cohort Study was established in 1983 as a prospective observational cohort study of HIV infected patients. From 1983 to 1998, the study captured data from 80% of all HIV-infected cases and all notified AIDS cases in the state of Western Australia. Comprehensive demographic and clinical data was and is collected at outpatient and in-patient visits by medical staff and entered into an electronic database. Start and stop dates of all antiretroviral treatments are recorded. Routine laboratory test results are automatically downloaded from the laboratory directly into the cohort database. Data from a maximum of 473 cohort subjects with HLA and viral sequence data were analysed in logistic regression models.

HLA genotyping: All HLA-A and HLA-B broad alleles were typed by microcytotoxicity assay using standard NIH technique. For this study, 51 HLA-B5 individuals and 57 HLA-B35 individuals had HLA-B sequence amplified using primers to the first intronic dimorphism as previously described (see for example N. Cereb and S. Y. Yang, Tissue Antigens 50, 74-76 (1997)) and products were sequenced by automated sequencing. HLA-DRB1 alleles were typed by sequencing using previously reported methods (see for example, D. Sayer et al., Tissue Antigens 57, 46-54 (2001)).

HIV-1 RT sequencing: HIV-1 DNA was extracted from buffy coats (QIAMP DNA blood mini kit; Qiagen, Hilden, Germany) and codons 20 to 227 of RT was amplified by polymerase chain reaction. A nested second round PCR was done and the PCR product was purified with Bresatec_purification columns and sequenced in both forward and reverse directions with a 373 ABI DNA Sequencer. Raw sequence was manually edited using software packages Factura and MT Navigator (PE Biosystems).

Quantitative HIV RNA assay: The viral load assay used until November 1999 was the HIV Amplicor™ (Roche, Branchburg, USA, lower limit of detection 400 copies/mL). The Roche Amplicor HIV monitor Version 1.5, Ultrasensitive, lower limit of detection 50 copies/mL was used thereafter. Viral load assays were routinely performed at least three monthly in all patients.

Statistical analysis: Using the WA HIV Cohort Study database to facilitate analyses based on Fishers exact tests and logistic regression models standard formulae were used for power calculations (see for example J. H. Zar, in Biostatistical Analysis, Bette Kurtz, Ed. (Prentice-Hall International, New Jersey, 1984), chap. 22.11).

Individual covariates were then assessed separately for association with polymorphism at the amino acid position under consideration using Fisher's exact test, and only those with univariate P-values≦0.1 were included in further analyses. If the number of covariates selected by this method exceeded 10% of the patient numbers a forward stepwise procedure based on standard logistic regression was used to reduce the number to 10% and standard backwards elimination used until all covariates had a P-value≦0.1.

For example, covariates were assessed separately for association with I135 using Fishers exact test, and only those with univariate P-values≦0.1 were included in further analyses. The removed alleles were A1, A2, A3, A9, A11, A19, A28, B7, B8, B13, B14, B15, B16, B21, B22, B27 and B35.

Since the number of covariates selected at position I135 was less than 10% of the number of patients, no forward selection was needed. A standard backwards elimination was then carried out at position I135. The covariant with the largest P-value was removed and the logistic model refitted. This was repeated until all covariates had a P-value less than 0.1, thus removing HLA alleles B12, B17 and B40.

To accommodate relatively small samples in some of the logistic regressions, exact P-values were based on randomisation tests rather than the usual large sample approximations (see for example F. L. Ramsey and D. W. Schafer, in The statistical sleuth. A course in methods of data analysis, (Duxbury Press, 1997), chap. 2). In this procedure covariate sets were randomly permuted amongst the patients and the standard test values for association with polymorphism calculated for each permutation. This procedure generated 1000 random permutations for each model and based the P-value on the appropriate percentage of test values more extreme than that pertaining to the actual data. P-values≦0.05 were considered to be significant using this method.

For Example, at Position I135, Alleles HLA-A10 and -B18 were Removed, Leaving HLA-B5 as the Significant Association with I135.

Analyses were conducted to determine the probability of finding by chance at least fifteen significant positive associations within corresponding known CTL epitopes. If significant associations were occurring randomly across residues, the probability that an HLA association would occur within the known CTL epitope restricted to that allele equates to the relative proportion of all residues falling within the epitope. The total number of significant associations within known epitopes is then a sum of non-identical binomial variables, whose distribution can be evaluated via simulation, for example. Only 4.27 significant positive associations within known epitopes were expected based on the random hypothesis compared with the 15 observed (approximate P-value<0.001).

Correction factors for multiple comparisons were generated as described later and corrected exact P-values were determined by the function: 1−(1−P)^{x} where x=correction factor. The overall P-value for all associations at all positions was obtained by considering the extremeness of the sum of the individual tests at each position relative to the values of this sum obtained from the randomisation data sets.

For the Cox proportional hazards models of viral load, HLA associations had to have at least four individuals representing HLA allele versus non-HLA allele, with polymorphisms and without to be included (n=106). The viral load measured closest to first pre-treatment HIV-1 RT sequencing was used.

Polymorphism in HIV-1 RT Amino Acid Sequence is Constrained by the Functional Importance of Residues

To determine whether polymorphisms in HIV-1 RT sequences in the study population were distributed randomly or occurred at preferred sites, the population consensus sequence was used as a reference sequence and was determined by assigning the most common amino acid at each position from 20 to 227 (numbering system as in reference B. T. M. Korber et al., HIV Molecular Immunology Database 1999 (Theoretical Biology and Biophysics, New Mexico, 1999)) of all first HIV-1 RT amino acid sequences prior to any antiretroviral therapy (n=185). This population consensus sequence matched the lade B reference sequence HIV-1 HXB2 (L. Ratner et al., Nature 313, 277-284 (1985)) at all positions in RT except 122 (lysine instead of glutamate) and 214 (phenylalanine instead of leucine). The percentages of patients with a different amino acid in their own first pre-treatment HIV-1 RT sequence to that of consensus sequence was calculated for each residue. The relationship between this polymorphism rate and the functional characteristics (stability, functional, catalytic or external) known for amino acids between positions 95 to 202 in HIV-1 RT was examined.

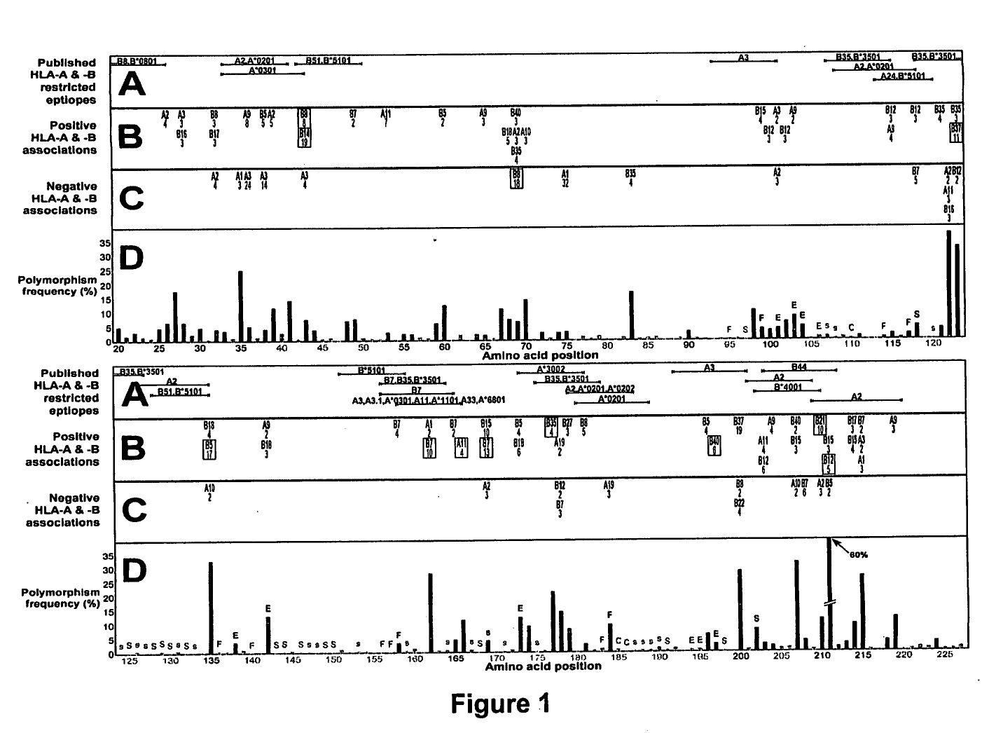

The rate of polymorphism at single residues was highly variable, ranging from 0% to 60% and appeared to correlate with the expected viral tolerability of change at that site (FIG. 1). For example, the polymorphism rates at the three critical catalytic residues in HIV-1 RT (0.53%), stability residues (n=37, 1.06%) and functional residues (n=11, 3.05%) were lower than at external residues (n=10, 5.95%) (P-0.0009, Wilcoxon).

Polymorphism of Residues within and Proximate to Known and Putative CTL Epitopes in HIV-1 RT are HLA Class I Allele Specific

As antigen specific CTL responses are HLA class I restricted, polymorphisms in HIV-1 RT that were the result of CTL escape mutation were examined to determine whether they would be HLA class I allele-specific across the population and would be in residues within or proximate to CTL epitopes. The relationship between HLA-A and HLA-B broad alleles (as explanatory covariates) and polymorphism in HIV-1 RT (as the outcome or response variable) in multivariate logistic regression models was therefore examined. The most recent HIV-1 RT sequence in each patient was used in these analyses (n=473). Single amino acid residues in HIV-1 RT were examined in separate models. An individual model at one residue determined the statistical significance of association(s) between the covariates (HLA alleles) and the outcome (polymorphism at that residue only) and gave odds ratios (ORs) for associations.

The statistical power to detect the effect of any individual HLA allele in these models depended on the frequency of the allele in the population and the frequency of polymorphism at the amino acid position being examined. An initial power calculation was performed for each position to determine for which alleles there was a reasonable power to detect an association if it existed (at least 30% power to detect an OR>2.0 or <0.5). Only those HLA alleles that had a univariate association with polymorphism with P≦0.1 were examined at each viral residue (one to ten HLA alleles, mean 3.15 at 72 positions) in subsequent analyses. Final covariates in the logistic regression models also withstood a standard forward selection and backwards elimination procedure. Permutation tests based on the logistic models were used to determine the exact P-values for associations (F. L. Ramsey and D. W. Schafer, in The statistical sleuth. A course in methods of data analysis, (Duxbury Press, 1997), chapter 2).

HLA alleles with less than 30% power were removed. The removed alleles at position 135 were A31, A36, B42, B55, B56, B58 and B61. It is important to note that there was less power to detect negative associations than positive associations. For example, at the mean HLA frequency of 10.9 and mean polymorphism rate of 4.0%, there was 30% power to detect an OR of 2.0 (i.e. a positive association) but only 5.6% power to detect an equivalent negative OR of 0.5.

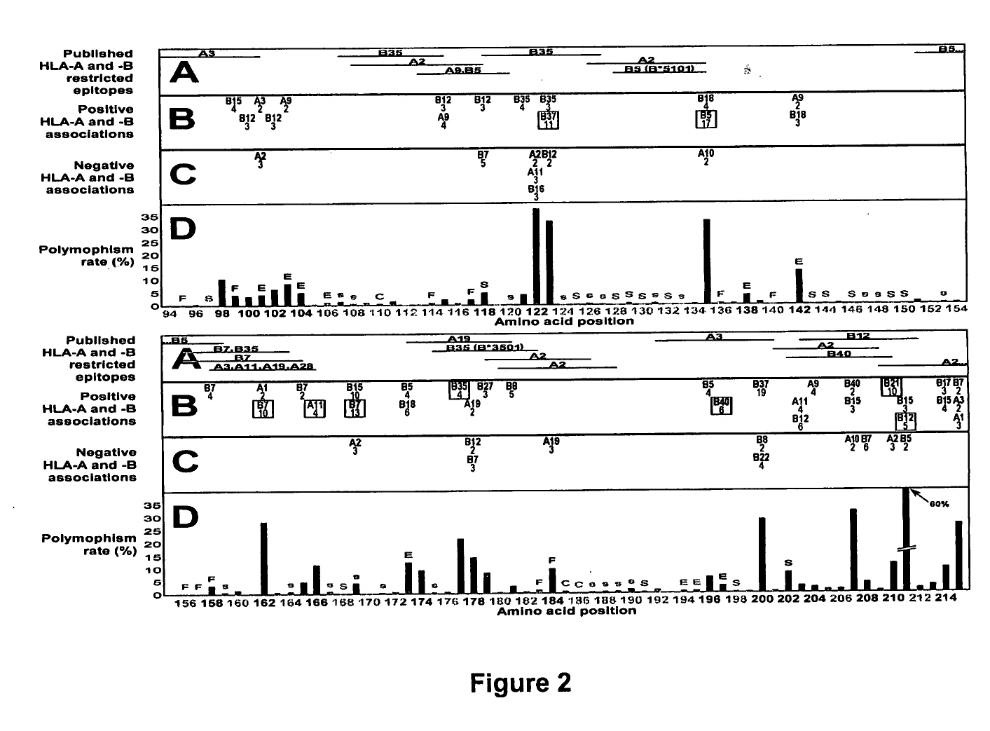

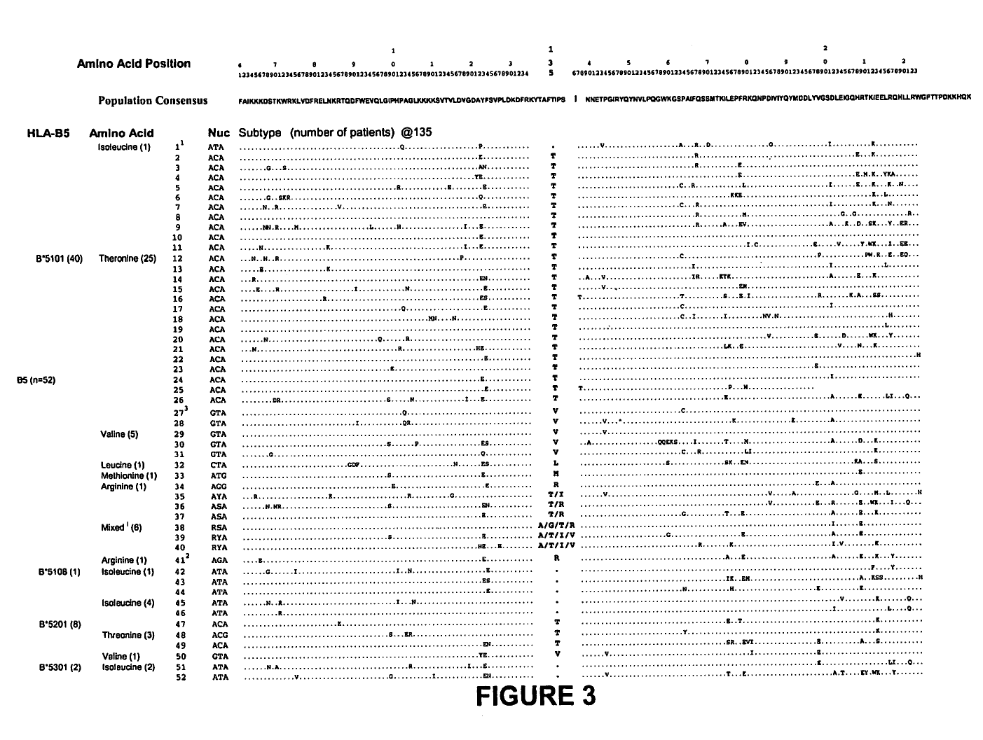

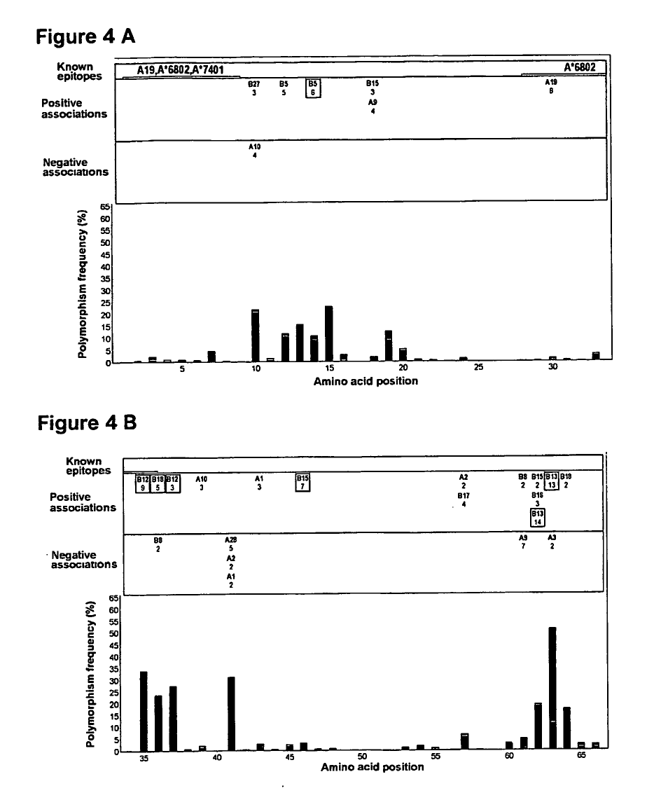

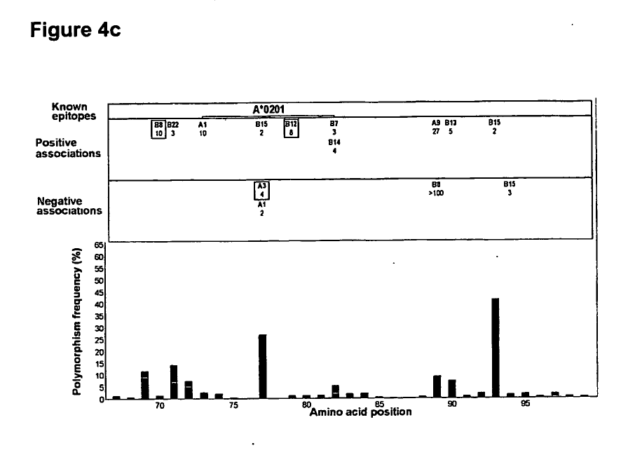

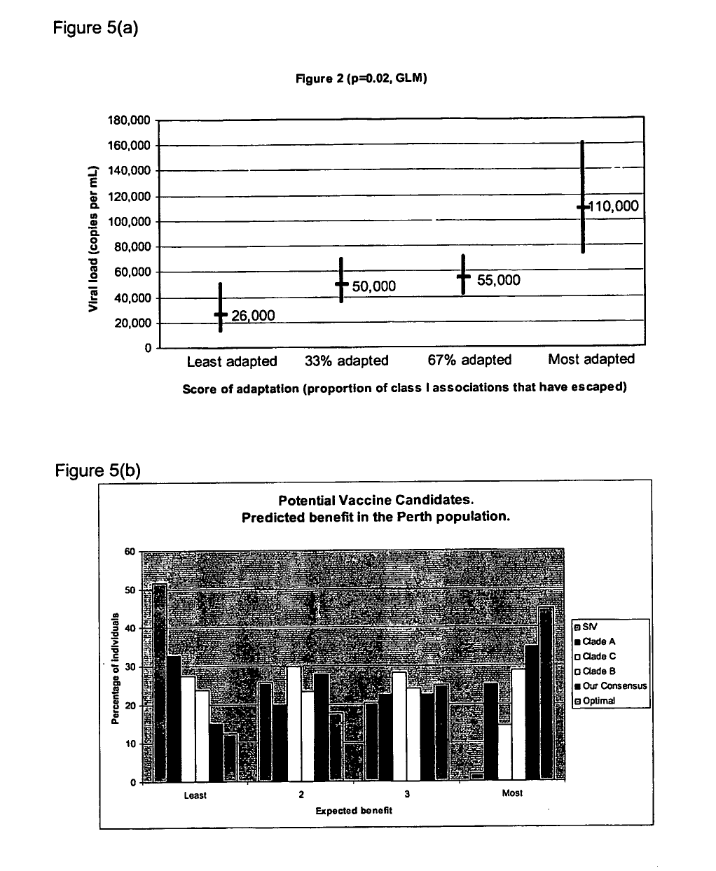

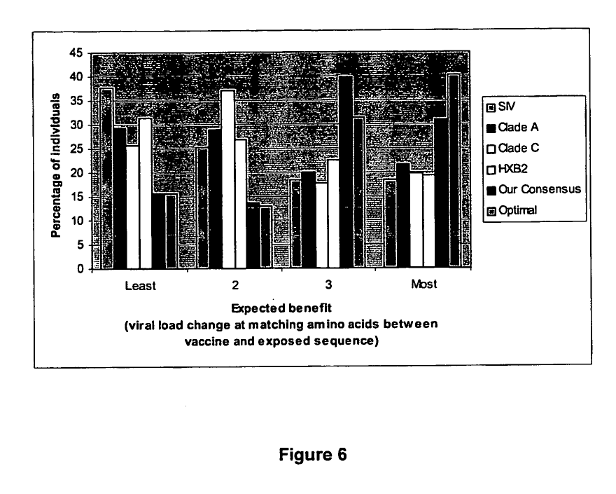

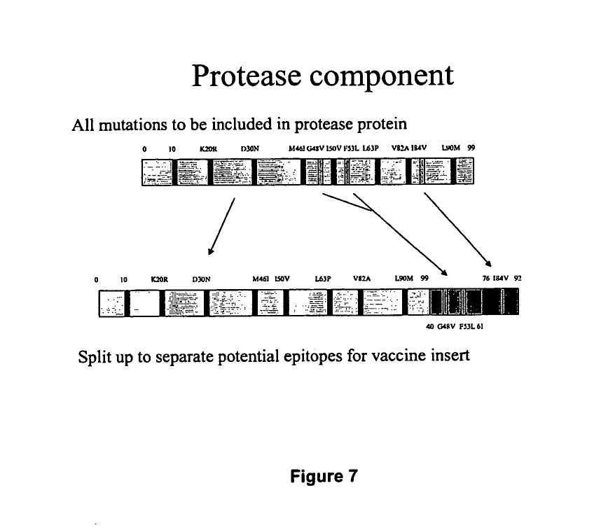

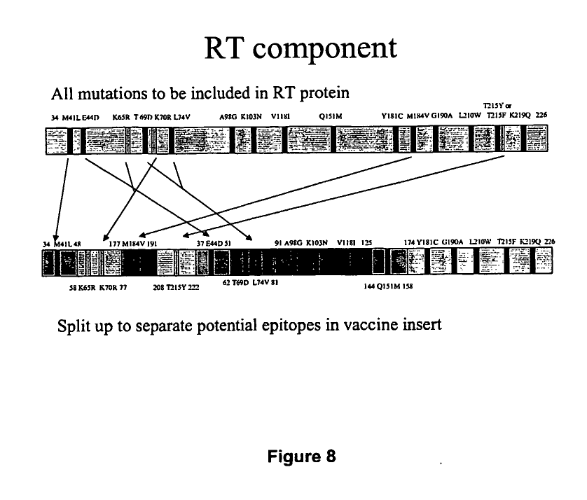

The results of all the individual models were plotted together on a map of HIV-1 RT amino acid sequence from position 20 to 227 (FIG. 2). There were 64 positive associations (ie OR>1) between polymorphisms of single residues in HIV-1 RT and specific HLA-A or -B alleles (P≦0.05 in all cases) (FIG. 2, Box B). Polymorphisms specific for a particular HLA allele clustered along the sequence. For example, HLA-B7 was associated with polymorphism at positions 158 (OR=4), 162 (OR=10), 165 (OR=2) and 169 (OR=13), which are all within or flanking the known HLA-B7 restricted CTL epitope RT(156-165) (C. M. Hay et al., J Virol 73, 5509-5519 (1999); L. Menendez-Arias, A. Mas, E. Domingo, Viral Immunol 11, 167-181 (1998); C. Brander and B. D. Walker, in HIV molecular immunology database, B. T. M. Korber et al., Eds. New Mexico, (1997)). There was also clustering of associations for HLA-B12 (at positions 100 and 102, 115 and 118, 203 and 211), HLA-B35 (121 and 123), HLA-B18 (at 135 and 142), and HLA-B15 (at 207, 211 and 214).

Fifteen HLA class I allele-associated polymorphisms (FIG. 2, Box B, shown in grey text) occurred at residues within the 29 CTL epitopes that are characterised, published and known to be restricted to those alleles. Four of these residues (101,135, 165 and 166) were at primary anchor positions within CTL epitopes (HLA-A3 (C. Brander and P. J. R. Goulder, in HIV Molecular Immunology 2000, B. T. M. Korber et al., Eds. (Theoretical Biology and Biophysics, New Mexico, 2000), chap. Part 1. Review Articles), HLA-B51 (L. Menendez-Arias, A. Mas, E. Domingo, Viral Immunol 11, 167-181 (1998); N. V. Sipsas et al., J Clin Invest 99, 752-762 (1997))/HLA-B*5101 (H. Tomiyama et al., Hum Immunol 60, 177-186 (1999)), HLA-B7 (C. M. Hay et al., J Virol 73, 5509-5519 (1999); L. Menendez-Arias, A. Mas, E. Domingo, Viral Immunol 11, 167-181 (1998); C. Brander and B. D. Walker, in HIV molecular immunology database, B. T. M. Korber et al., Eds. New Mexico, (1997)) and HLA-A11 (Q. J. Zhang, R. Gavioli, G. Klein, M. G. Masucci, Proc Natl. Acad. Sci U.S.A 90, 2217-2221 (1993)) restricted respectively) where mutation could abrogate binding to the HLA molecule. The remaining 11 associations were at non-primary anchor positions of published CTL epitopes. There were a further five HLA allele-specific polymorphic residues that flanked CTL epitopes restricted to the same HLA alleles (FIG. 2, shown in Black text). The residues at positions 26 and 28 that flank known HLA-A2 and HLA-A3 restricted epitopes were predicted proteosome cleavage sites (C. Kuttler et al., J Mol Biol 298, 417429 (2000)). If significant positive associations occurred randomly across residues only 4.18 would have been expected to fall within corresponding known CTL epitopes. The observed number of 15 was significantly higher than this (P<0.0004). Furthermore, an excess of associations over that expected was seen for ten of the 11 HLA specificities with epitopes in this segment of HIV-1 RT.

A final set of analyses was conducted to identify which of these significant HLA associations would remain significant after a correction for the effective number of independent comparisons made over the entire analysis. HLA genotypes were randomly reassigned amongst individuals and the previously described analysis was run 1000 times to determine the number of false positive associations expected by chance alone for each HLA allele. The average number of P-values≦0.05 obtained was multiplied by 20 (ie 1/0.05) to estimate the effective number of independent tests carried out as a correction factor for multiple comparisons for each HLA allele. Correction factors ranged from 5.0 (HLA-B37) to 92.2 (HLA-B7) for positive associations and 0.8 to 42.8 for negative associations. There were 14 associations that still had a P≦0.05 following this correction (FIG. 2, HLA associations in boxes).

The randomisation data sets were also used to generate an overall test of significance, taking multiple comparisons into account, of all HLA associations at all positions across all models. This test had a P-value of <0.001.

Molecular HLA Sub-Typing can Increase Strength of Association Between Polymorphism and HLA Alleles.

Serologically defined HLA class I alleles have subtypes, defined by high resolution DNA sequence based typing, that have amino acid sequence differences in the peptide binding regions that influence epitope binding. For these alleles, it would be expected that CTL escape mutation would be more closely associated with the molecular subtype than with the broad HLA allele. As examples, two strong associations with broad HLA alleles with well-represented splits, at sites within known CTL epitopes, and where the HLA restriction of the epitope at the molecular level was known were examined. Polymorphism at position 135 (I135x, where I is the consensus amino acid isoleucine and x is any other amino acid) associated with presence of HLA-B5 was the strongest positive HLA association at a residue within a published epitope (OR=17, P<0.001). D177x, within an epitope specifically restricted to the HLA-B*3501, was associated with HLA-B35 (OR=4, P<0.001) (FIG. 2).

I135x is Associated with HLA-B*5101

Isoleucine is the amino acid at position 135 of the consensus HIV-1 RT sequence. It is the eighth amino acid and anchor residue of a known 8mer HLA-B5 (*5101) restricted CTL epitope, RT(128-135 IIIB). Six of the other seven amino acid residues of the epitope are critical stability residues for the RT protein and are relatively invariant in the cohort (FIG. 1, FIG. 2). Of all 52 HLA-B5 positive patients, 44 (85%) had a substitution of isoleucine at position 135. Of the 421 non-HLA-B5 individuals, only 123 (29%) had this change (P<0.0001, Fisher's exact test).

DNA sequencing to subtype all 52 individuals in the cohort with the HLA-B5 allele was undertaken (FIG. 3). One HLA-B5 patient did not have sufficient DNA sample to perform high resolution HLA typing. Forty of the remaining 51 HLA-B5 patients were of the HLA-B*5101 subtype. All but one of these 40 HLA-B*5101 patients (98%) had I135x (I135T in 25 cases, I135V in 5 cases, I135L/M/R or mixed species in the remaining 9 cases). In contrast, only 127 of the 432 (29%) non-HLA-B*5101 patients in the cohort had I135x (P<0.0001, Fisher's exact test). For the most common substitution, from isoleucine to threonine, the predicted half time of dissociation score for the mutant epitope (TAFTIPST) is 11 compared with 440 for the consensus sequence (TAFTIPSI), indicating that binding to the HLA molecule in vivo is abrogated. This substitution has been shown to necessitate a hundred-fold increase in the peptide concentration required to sensitise target cells for 50% lysis (SD₅₀) by CTLs in vitro (N. V. Sipsas et al., J Clin Invest 99, 752-762 (1997)). The less common isoleucine to valine substitution at position 135 has been associated with a ten-fold increase in SD₅₀ compared with consensus epitope (N. V. Sipsas et al., J Clin Invest 99, 752-762 (1997)).

The single HLA-B*5101 patient who was not different to consensus at position 135 was a patient who had highly active antiretroviral therapy (HAART) administered during acute HIV seroconversion. The patient had presented within days of virus transmission with plasma HIV RNA concentration (viral load) of 6.5 log copies/mL and a negative HIV antibody test. He had no symptoms of seroconversion illness. After HMRT was started, viral load progressively decreased to undetectable levels over the next six months, and has remained undetectable on treatment for a further ten months until the present time.

The one patient with the HLA-B*5108 subtype, and four of eight patients with the HLA-B*5201 subtype did not have I135x, suggesting that these subtypes may not bind the RT(128-135 IIIB) epitope. Both subtypes differ from HLA-B*5101 by only two amino acids (HLA-B*5108 at positions 152 and 156, HLA-B*5201 at positions 63 and 67, of HLA amino acid sequence) (IMGT/HLA sequence database; http://www.ebi.ac.uk/imgt/hla). The remaining two patients were shown to be HLA-B*5301 by sequencing (FIG. 3).

D177x is Associated with HLA-B*3501

The HLA-B35 subtype HLA-B*3501 only differs from HLA-B*3502, -B*3503, -B*3504 by one or two amino acids in the peptide binding region and yet the different epitope specificities of these subtypes have a striking effect on risk of clinical progression of HIV-1 infection. The epitope RT(175-183) binds to HLA-B*3501 and contains a binding motif that is distinct to that predicted for other HLA-B35 subtypes (http://www.uni-teubingen.de/uni/kxi/). Of 57 HLA-B35 positive individuals in the study population, 26 (46%) had D177x compared with 84 of 416 (20%) non-HLA-B35 individuals (P<0.0001, Fisher's exact test). However, there were 19 of 33 (58%) HLA-B*3501 patients that had D177x compared with 86 of the 440 (20%) non-HLA-B*3501 patients (P<0.0001, Fisher's exact test). Thus, the univariate relative risk of polymorphism increased from 2.7 to 4.7 after the molecular subtype of HLA-B35 was considered. This analysis was repeated for other HLA-B35 associated polymorphisms in HIV-1 RT, I69x, D121x and D123x and in all cases, the association was strengthened by considering molecular subtypes of HLA-B35.

HLA-Specific Polymorphisms in HIV-1 RT are Selected Over Time

To determine whether selection of HLA-specific polymorphisms over time was demonstrable, the amount of HLA-specific variation present in the most recent HIV-1 RT sequence with the first sequence for all individuals was examined. For 61 of 64 HLA-specific polymorphisms, the number of individuals with a specific amino acid polymorphism increased over time and under observation. In 52 of these cases, the increase was significantly greater in those with the HLA allele associated with the polymorphism, compared with all others without the allele (P-0.0008, sign test) as shown in Table 1.

TABLE 1

| Polymorphism | Number (n) | P-value (sign test) |
|---|---|---|
| HLA-specific polymorphisms | 64 | P < 0.0001 |
| HLA-specific polymorphisms that | 61 | P < 0.0001 |
| increase from first to last HIV-1 RT | | |
| sequences | | |
| HLA-specific polymorphisms that | 52 | P < 0.0001 |
| increase from first to last HIV-1 RT | | |
| sequences in those with the | | |
| corresponding allele compared with all | | |
| others | | |

HLA-Specific Polymorphisms in HIV-1 RT are Associated with Secondary Changes at Other Positions.

Primary CTL escape mutation in an HIV-1 p24 epitope has been shown to induce possible compensatory mutations in the virus. To determine whether the secondary or compensatory changes accompanying primary (putative) CTL escape mutation were evident at a population level, polymorphisms were included at all ‘other’ positions in HIV-1 RT, along with HLA alleles, as covariates in all multivariate logistic regression models. All but two of the 64 positive HLA-specific polymorphisms were also associated with one or more polymorphisms at other positions.

Negative Associations Between HIV-1 RT Polymorphisms and HLA Alleles.

In the multiple logistic regression models described earlier, there were 25 residues at which polymorphism was HLA-specific but with an OR<1, indicating a ‘negative’ association. For example, change from consensus amino acid at positions 32, 101, 122, 169, and 210 of HIV-1 RT was negatively associated with presence of HLA-A2 (P≦0.05 in all cases). This means that HLA-A2 individuals were significantly less likely to vary from the consensus at these sites compared with all non-HLA-A2 individuals in the cohort. The negative ORs were inversed (1/OR) to give a value >1 for the odds of not having a polymorphism (FIG. 2, Box C). HLA-A2 is the most common HLA-A allele in our cohort and had five of the 25 negative associations (compared with three of the 64 positive associations). Similarly, individuals with HLA-B7 were more likely to have the consensus amino acid at positions 118, 178 and 208 compared with non-HLA-B7 individuals. According to this analysis there was less power to detect negative associations than positive associations. For example, at the mean HLA frequency of 10.9 and mean polymorphism rate of 4.0%, there was 30% power to detect an OR of 2.0 (ie a positive association) but only 5.6% power to detect an equivalent negative OR of 0.5.

HLA-Specific Polymorphisms in HIV-1 RT are Associated with Higher Pre-Treatment Viral Load.

As HIV-1 viral load has been shown to be inversely proportional to HIV-specific CTL responses, studies were undertaken to determine whether the presence of putative CTL escape mutations was associated with increased viral load. Individual HLA-specific polymorphisms were selected for examination. A polymorphism at an anchor residue was considered. HLA-A11 associated K166x is at the anchor position of an HLA-A11 epitope RT(158-166 LAI) and HLA-A11 groups with and without the polymorphism had sufficient numbers for comparison. To exclude effects of antiretroviral therapy, only patients with HIV-1 RT sequence and viral load results prior to treatment were analysed. The closest pre-treatment viral load measurement taken after the HIV-1 RT sequencing, was compared between all groups. In HLA-A11 individuals (n=19), the median pre-treatment viral load was 5.54+/−0.46 log cps/mL plasma (median+/−SD) in those with K166x (n=4) compared with 4.31+/−0.82 log cps/mL, in those without K166x (n=15, P=0.045, Wilcoxon). Median viral load in HLA-A11 individuals without K166x was not significantly different from that of all non-HLA-A11 individuals (data not shown).

A second putative CTL escape mutation within a CTL epitope but not at a primary anchor position showed a similar effect. The median pre-treatment viral load in HLA-B7 patients with S162x (n=18) was significantly higher (5.41+/−1.04 log cps/mL) than in those without S162x (n=15, 4.57+/−0.83 log cps/mL, P=0.046, Wilcoxon). For both HLA-A11 and HLA-B7 groups, the mean CD4 T cell count and percentage of individuals with AIDS at baseline was not significantly different between those with and those without these putative CTL escape mutations.

A global analysis of factors influencing viral load at a population level was then conducted. A Cox proportional hazards model was carried out in which pre-treatment viral load was the outcome and all HLA alleles and HLA-specific polymorphisms were discrete covariates. When HLA alleles and polymorphisms were included as interaction terms (i.e. a polymorphism and it's positively associated HLA allele, or consensus amino acid and the negatively associated HLA allele) the overall significance value of the model improved. The former model had a log likelihood of −32.0765 with 40 degrees of freedom and the latter model had a log likelihood of −15.4165 with 25 degrees of freedom. The improvement in the model was calculated using a chi square distribution with a value of two times the difference in log likelihood values with degrees of freedom (33.32−χ(15), giving a P-value of 0.004). This suggested that the presence in individuals of viral CTL escape mutations as putatively identified in these analyses, explained the viral load variability in the population to a greater extent than either HLA alleles or viral polymorphisms per se.

HLA-DRB1 Allele Specific Polymorphism in HIV-1 RT-Evidence of Viral Escape from Anti-HIV CD4 T Helper Cell Responses?

We repeated logistic regression models of polymorphism incorporating HLA-DRB1 broad alleles as covariates, along with HLA-A and -B alleles and polymorphisms at other positions. Only patients in the cohort with DRB1 alleles defined by DNA sequence based typing were included in this analysis (n-294). There were 13 sites of polymorphism between positions 20 and 227 that were significantly associated with HLA-DRB1 alleles. Only five T helper cell epitopes have been mapped within this segment of HIV-1 RT (A. S. de Groot et al., J of Infectious Diseases 164, 1058-1065 (1991); S. H. van der Burg et al., J Immunol 162, 152-160 (1999); F. Manca et al., J of Acq. Imm. Def. Syn. & Hum. R 9, 227-237 (1995); F. Manca et al., Eur J Immunol 25, 1217-1223 (1995)) and only one, RT(171-190), has been assigned HLA-DRB1 allele(s) specificity (S. H. van der Burg et al., J Immunol 162, 152-160 (1999)). Four of the five known CD4 T helper cell epitopes encompassed sites of HLA-DRB1 allele-specific polymorphism found in the models described herein. These analyses did not detect an HLA-DRB1 association within RT(171-190). There were 10 HLA-DRB1 associated polymorphisms that were not within known T helper cell epitopes.

Discussion

According to these analyses, HIV-1 RT sequence is relatively conserved among isolates however, even in a stable, geographically isolated population of HIV-1 infected persons there is sequence diversity of HIV-1 RT. The population consensus sequence was used in this study as the presumptive wild-type sequence best adapted to the population as a whole and was almost identical to the lade B reference sequence HXB2-RT. Yet, within the study population, variation from this consensus sequence was evident even in a segment of HIV-1 RT. Findings presented herein suggest that this diversity is the net result of at least two competing evolutionary pressures selecting for or against change at each amino acid. Foremost is the need to maintain functional integrity of the virus. Within the bounds of this fundamental constraint, a strong predictor of viral polymorphism appears to be host HLA.

There were 64, often clustered, polymorphisms in HIV-1 RT associated with specific HLA-A or HLA-B alleles. Polymorphisms occurred at sites that were within or proximate to published CTL epitopes, and correlated with the HLA alleles to which these epitopes are known to be restricted. This correlation was itself highly statistically significant and several associations still remained significant after rigorous correction for multiple comparisons across the whole analysis. The detailed features of specific examples, such as HLA-B*5101 associated I135x, were highly suggestive of CTL escape mutation affecting HLA-peptide binding. Polymorphisms at non-primary anchor residues of CTL epitopes, such as HLA-B*3501 associated D177x, HLA-B7 associated S162x and others may confer a survival advantage to the virus by disrupting T cell receptor-peptide recognition, epitope processing from precursor protein or by inducing antagonistic CTL responses. The five HLA-specific polymorphisms at residues flanking CTL epitopes may indicate viral escape by disruption of proteosome peptide cleavage. This form of escape has been particularly difficult to identify by standard techniques that use only the epitope peptide to measure CTL responses. HLA-specific polymorphisms increased over time, were associated with secondary changes at other positions and were predictive of viral load at a population level. The effect of single residue changes on viral load is especially striking given that there may be a polyclonal immune response against epitopes in other HIV-1 genes and other independent influences on viral load such as CCR5 polymorphism. Taken together, these data suggest that the HLA-specific polymorphisms identified herein in HIV-1 RT represent the net effects of in-vivo CTL escape mutation in individuals. By implication, those polymorphisms not within published CTL epitopes may indicate where new or putative CTL epitopes are located. The HLA associations that are very strong (with high OR), and which are clustered or remain significant after correction for multiple comparisons (FIG. 2 shown in boxes) are those most likely to represent viral escape mutations in CTL epitopes that are yet to be defined.

CTL escape mutation has been well characterised in individuals with HLA-B8 (most commonly), HLA-B44, HLA-B27, HLA-A11 and HLA-A3, who may have been more escape-prone because of narrow range, oligoclonal CTL responses. These data suggest that CTL escape mutation is common and widespread, selected by responses restricted to a much wider range of HLA alleles than has been studied in individual cases. Though many HLA-specific polymorphisms increased over time in this study, some were present in first pre-treatment HIV-1 RT sequence and could reflect viral founder effects, have been variants selected at transmission or during the early CTL response of acute infection (FIG. 1). The single HLA-B*5101 patient without I135x was distinguished by use of HAART in acute infection whilst highly viremic. This patient presented in the first days of infection with no symptoms, suggesting he had not yet mounted a CTL response. Presumably, the immune selection pressure was reduced or eliminated, arguing that I135x is selected during the acute CTL response, rather than selected at transmission or in chronic infection in HLA-B*5101 individuals. Protection from CTL escape variants may contribute to the effect of HMRT in acute HIV infection leading to stronger chronic inhibitory CTL responses which, to date, has been largely attributed to preservation of HIV-1 specific CD4 T cell help.

HLA alleles were also associated with lack of polymorphism at certain residues, including at residues without functional constraint (FIG. 2) and these associations contributed independently in a comprehensive model of viral load. Unlike positive immune selection causing demonstrable escape over time in individuals, negative immune selection favours preservation of wild-type virus in vivo and so could only be evident at a population level. It is possible that consensus or wild-type virus is primordially adapted to the CTL responses that have most often been encountered (that is, those restricted to the most common or evolutionary conserved HLA alleles in the host population). For HIV-1, this may account, at least in part, for HIV-1 lade differences. Population adaptation could also explain why selection of escape polymorphisms in CTL epitopes restricted to the common allele HLA-A*0201 was not demonstrated in studies that have argued against an important role for immune escape and even why surprisingly few HLA-A2 and HLA-A1 restricted epitopes have been mapped in HIV-1. Furthermore, studies of HIV-1 exposed seronegative individuals suggest that CTL responses can alter viral infectivity and susceptibility to established primary HIV-1 infection. The HLA class I alleles associated with natural HIV-1 resistance or susceptibility appear to differ between racially distinct populations. To some extent this may reflect differences in the HLA alleles that are common in different populations and the degree to which a ‘population-adapted’ consensus virus can adapt to the individual.

Demonstration herein of 13 HLA-DRB1 specific polymorphisms in HIV-1-RT (adjusted for HLA-A and HLA-B associations and secondary polymorphisms) may lend support to the possibility of CD4 T helper escape mutation in human HIV-1 infection. Relatively few T helper cell epitopes in HIV-1 RT are published and their HLA-class II restrictions are not defined, so it is difficult to assess whether these results are consistent with T helper selection of escape mutation. However, HLA class II restricted CD4 T helper responses have a central role in HIV-1 control and there are several reported associations between HLA class II alleles and HIV disease susceptibility and progression including after HMRT.

The population-based approaches in this study reveal how both positive and negative selection forces compete at single residues to drive primordial and current viral evolution in vivo. These results are especially notable considering the factors that reduce the likelihood of observing significant HLA associations in such analyses. Firstly, the power to detect associations is not constant for all HLA allele/viral residue combinations. Large numbers of individuals would be needed to observe any polymorphism at residues under immune pressure to mutate but with strong functional constraint, or any associations with HLA alleles that are rare. The use of formal power calculations identifies those HLA associations that cannot be excluded and would need larger data sets to be examined. Secondly, the molecular subtype of an HLA allele predicts its binding properties in vivo, as shown by the enhancement of associations between HLA-B5 and I135x, and HLA-B35 and D177x by high resolution HLA typing. Other alleles with multiple splits of similar frequency (e.g. HLA-A10 or HLA-A19) may have had associations that were not detected because only broad alleles were considered. Furthermore, molecular splits that have opposing effects at the same viral residue would negate any association with the broad allele. Finally, published epitopes are more likely to be in conserved regions, as studies tend to use laboratory reference strains as target antigens and conserved regions are more likely to have measurable immune responses in vivo. This approach, in contrast, preferentially detects putative immune epitopes in variable regions, making it complementary to standard epitope mapping methods. Insufficient patient numbers, lack of molecular based HLA typing and lack of known epitopes in conserved regions could all account for the immune epitopes in which ‘expected’ HLA-specific polymorphisms were not detected, and could mean that the strength (OR) of the demonstrated associations were underestimated in some cases.

The generation of chance associations as a result of comparisons made with multiple covariates (HLA alleles) and at multiple residues potentially hampers such analyses, though power calculations and other screening procedures considerably restrict the number of alleles and positions that are examined. The degree to which P-values generated within multivariate logistic regression models are corrected for the number of residues examined will then depend on the size of the gene(s) that has been arbitrarily chosen for study. With such correction, the approach will lose power to detect associations in direct proportion to the size of the gene region selected, decreasing false positive associations (higher specificity) but at the cost of losing true positive associations (lower sensitivity). These analyses of HIV-1 RT provided a gradation of P-values uncorrected for multiple comparisons, reflecting a gradation in strength of associations. Independent biological validation, rather than statistical means, will best determine what p-value cut-offs are optimal for either sensitivity or specificity. If correction is to be made (for high specificity) the randomisation procedure undertaken allows the number of effective independent comparisons in the entire analysis to be estimated. Those HLA associations with P-values that withstand this rigorous correction have been highlighted by these methods (FIG. 2, associations in boxes). These highly robust associations represent the starting point to map new epitopes in HIV-1 RT.

In terms of the known associations between certain HLA and HIV-1 disease progression, HLA allele frequencies influence adaptation of ‘wildtype’ HIV-1 at a population level. However, in-vivo evolution proceeds within individuals of diverse HLA. This analysis shows that it is the presence of HLA alleles with their corresponding HLA-specific viral polymorphisms (or consensus) that is more predictive of viral load than the HLA alleles alone. It has also been suggested that it is the breadth of CTL responses that determines the risk of viral escape and hence, clinical progression. Narrow monospecific responses, as seen in HLA-B*5701 long term non-progressors, can be protective but may also increase risk of viral escape in individuals with the deleterious HLA allele, HLA-B8. Increasing heterozygosity of the three HLA class I loci, which would predict broader polyclonal responses, has been shown to predict slow progression to AIDS. Successful viral CTL escape mutation depends on having low functional barriers to mutation at the appropriate residues, so it may be the balance struck between the breadth of host epitope-specific CTL responses and viral functional constraint at those epitopes that is important. Hence narrow CTL responses could be protective if directed against conserved epitopes, but not protective or harmful if directed against epitopes susceptible to variation. The ability to map both the range of putative epitopes and the observed polymorphism of the epitope in a population for many HLA alleles at once is thus very useful. Future analyses of HIV-1 RT should also incorporate reverse transcriptase inhibitors as covariates in the models to examine the interaction between drug-induced primary or compensatory mutation and HLA-associated primary or secondary polymorphism. If immune pressures and antiretroviral drugs compete at sites within viral sequence, a greater or lesser tendency to drug resistance and response may be seen in patients depending on their HLA genotype. Individualisation of antiretroviral therapy may be improved if synergistic or antagonistic interactions between immune pressure and drug pressure are better understood. Just as these methods have identified the location of putative immune epitopes in HIV-1 RT, candidate epitopes in other HIV-1 proteins or proteins from other microorganisms could be screened for in the same way and then confirmed using standard assays of epitope-specific immune responses in vitro or in vivo. In HIV envelope, effects associated with anti-HIV antibody responses, CCR5 and CXCR4 genotype and any other polymorphisms of genes encoding products targeting envelope proteins may also be considered.

### EXAMPLE 2

### Polymorphism in Both HIV-1 RT and Protease Amino Acid Sequence

In this study HIV-1 protease is examined using the methods described above. In particular the method examines whether, in both HIV-1 RT and protease, host CTL pressure and drug pressure may compete or synergise at specific sites, which then influence drug resistance pathways in ways unique to the individual of given HLA type.

Bulk HIV-1 RT and protease pro-viral DNA sequences obtained from 550 individuals with HIV-1 infection were analysed. Single amino acid positions were examined at a time. The consensus amino acid for each position was determined and compared against the amino acids present in each individual's autologous viral sequence at the corresponding position. A multivariate analysis for a single residue (for example, residue 184 of HIV-1 RT, methionine in consensus) was carried out in which the outcome of interest was the presence or absence of a specified polymorphism (Ml 84V) or alternatively, any variation from consensus (M184x). The statistical significance of association(s) between this outcome and covariates such as the antiretroviral drugs used by the individuals and/or their HLA types, were then determined. Using model selection steps as previously described, this process was repeated for every residue making up the full HIV-1 RT and protease proteins.

Study population: The study population was drawn from The Western Australian (WA) HIV Cohort Study which has been described elsewhere. Start and stop dates of all antiretroviral treatments are recorded. HLA-A and HLA-B genotyping has been routinely performed at first presentation since 1983. HIV-1 RT proviral DNA sequencing has been requested at first presentation (prior to treatment where possible) and during routine clinical management of antiretroviral therapy since 1995. HIV-1 protease sequencing was commenced in 1997. The total cohort in this study comprised 550 individuals. All had at least one HIV-1 RT sequence recorded and 419 individuals had protease sequence available for analysis.

Statistical methods: All analyses were performed as described above. The population consensus sequence for HIV-1 RT(20-227) and protease (1-99), with standard HXB2 numbering and alignment, was used as the reference sequence in all analyses. The population consensus sequence matched the clade B reference sequence HIV-1 HXB2 at all positions in HIV-1 RT except 122 (lysine instead of glutamate) and 214 (phenylalanine instead of leucine). In HIV-1 protease, consensus sequence differed at position 37 (asparagine instead of serine) and 63 (proline instead of lysine).

Power calculations were conducted to limit analyses to only those positions, drugs and HLA alleles for which there was at least 30% power to detect associations with OR>2 (positive associations) or <0.5 (negative associations) with p-value<0.05. Individual covariates were then assessed for univariate association with mutation/substitution, and discarded if p-values were >0.1 and then subjected to forward selection and backwards elimination procedures. Exact p-values were determined for each association. Finally, a randomisation or bootstrapping procedure was carried out to determine a correction factor for final (HLA) associations to adjust for multiple comparisons.

HLA genotyping: All HLA-A and -B broad alleles were typed by microcytotoxicity assay using standard NIH technique.

HIV-1 RT and protease sequencing: HIV-1 DNA was extracted from buffy coats (QIAMP DNA blood mini kit; Qiagen, Hilden, Germany) and codons 20 to 227 of RT was amplified by polymerase chain reaction. A nested second round PCR was done and the PCR product was purified with_Bresatec purification columns and sequenced in both forward and reverse directions with a 373 ABI DNA Sequencer. Raw sequence was manually edited using software packages Factura and MT Navigator (PE Biosystems).

Selection of Antiretroviral Drug Resistance Mutations in HIV-1 Sequence at a Population Level.

Only well characterised drug resistance mutations were selected for this examination. Among the 273 individuals in the cohort with pre-treatment HIV-1 RT sequences available, 12 (4.4%) contained HIV-1 RT primary and/or secondary mutations resistance mutations. Of 168 individuals with pre-treatment protease sequences available, 49 (29.2%) had protease primary resistance mutations. For those individuals with known seroconversion date (n=182), the mean time from seroconversion to time of first pre-treatment sequence was 5.7 years.

The pooled sequences of the whole cohort were then examined. 288 (52.4%) of these individuals had either past or current treatment with antiretroviral drugs, including NRTIs in 52.0%, NNRTIs in 8.2% and PIs in 16.4%. For each logistic regression model carried out for one position at a time, only the specific amino acid substitution characteristic of drug resistance was considered as the outcome. All sequential sequences for each individual were analysed, spanning a mean period of 1.9 years per person. The earliest presence of a resistance mutation was recorded as a positive outcome, all subsequent sequences were discarded and all drug exposures prior to the outcome were entered as covariates. The outcome was recorded as negative if mutation had not developed in any sequence.

Primary and/or secondary drug resistance mutations were detected in 33.6% of subjects in post treatment HIV-1 RT sequences. The mutations detected with sufficient frequency to be examined in the logistic regression analyses included M41L, D67N, K70R, L74V, K103N, Y181C/I, M184V, G190A/S, L210W, T215Y and K219Q/E, whilst K65R, 75, V108I, Q151M and P225H were only rarely or not detected (<4.0% of sequences) and therefore had little power to be examined. For all the resistance mutations examined, the drug(s) associated with selection of the mutation at a population level corresponded to those known to select for the mutation from other studies (Table 2). For example, use of lamivudine was associated with the development of M184V with an OR of 19 (p<0.001). Use of zalcitabine independently increased risk of developing M184V (OR=3, p=0.004). Positive associations between L74V or M184V and use of abacavir were not detected in the study population. There was inadequate statistical power to detect associations between use of delavirdine and mutations as this agent was rarely used.

TABLE 2
The amino acid substitutions in HIV-1 RT examined in models,
with their published causative antiretroviral agent(s) and those
associated with these substitutions at a population level in this
study. OR-odds ratio, ZDV-zidovudine, ddI-didanosine,
3TC-lamivudine, d4T-stavudine, ABC-abacavir, NRTI-nucleoside
analogue reverse transcriptase inhibitor, NNRTI-non-nucleoside
analogue reverse transcriptase inhibitor.

| Amino acid | | | | |
|---|---|---|---|---|
| substitutions | Published | Drug association(s) | | |
| examined in | primary drug | detected at a population | | |
| HIV-1 RT | association(s) | level in study cohort | OR | P-value |
| M41L | thymidine | ZDV | 3 | <0.001 |
| | NRTI | | | |
| D67N | ZDV? | ZDV | 10 | <0.001 |
| K70R | thymidine | ZDV | 2 | <0.001 |
| | NRTI | | | |
| L74V | ddI | ddI | 8 | <0.001 |
| | ABC | | | |
| K103N | NNRTI | nevirapine | 6 | <0.001 |
| | | efavirenz | 6 | <0.001 |
| Y181C/I | nevirapine | nevirapine | 9 | <0.001 |
| | delavirdine | | | |
| M184V | 3TC | 3TC | 19 | <0.001 |
| | ddC | ddC | 3 | 0.004 |
| | ABC | | | |
| G190A/S | nevirapine | nevirapine | 11 | <0.001 |
| L210W | ZDV | ZDV | 2 | 0.016 |
| T215 Y | thymidine | ZDV | 4 | <0.001 |
| | NRTI | | | |
| K219Q/E | ZDV | ZDV | 4 | <0.001 |

There were primary Pi resistance mutations (D30N, M46I/L, G48V, V82A/T/F, L90M) detected in 24.1% and secondary PI resistance mutations (L10I, I54V/L, A71V/T, 73, V77I, I84V, N88S) in 30.3% of individuals with post-treatment protease sequencing. All but two (D30N and nelfinavir, G48V and saquinavir) of the expected the associations between individual PIs and primary PI resistance mutations were evident in the study population (Table 3). There was inadequate statistical power to detect associations between use of amprenavir or lopinavir and mutations.

Selection of CTL Escape Mutations in HIV-1 Sequence at a Population Level

The models as described above were repeated for all amino acids in HIV-1 RT and protease and added the HLA-A and -B (broad) serotypes of all individuals as covariates, along with drug exposures. At those positions that were known primary or secondary drug resistance mutation sites, the characteristic drug resistance amino acid substitution was specified as the outcome. At all other positions, any non-consensus amino acid was the outcome.

TABLE 3
Amino acid substitutions in HIV-1 protease examined.
PI-protease inhibitor

| Amino acid | | | | |
|---|---|---|---|---|
| substitutions | Published | | | |
| examined in | primary drug | Drug association(s) | | |
| HIV-1 protease | association(s) | detected in study cohort | OR | P-value |
| L10I/R | secondary | indinavir | 2 | 0.005 |
| | broad PI | saquinavir | 3 | <0.001 |
| D30N | nelfinavir | ND | | |
| M46I/L | primary | indinavir | 3 | 0.006 |
| | indinavir | | | |
| G48V | primary | ND | | |
| | saquinavir | | | |
| I54V/L | indinavir | indinavir | 5 | <0.001 |
| A71V/T | secondary | indinavir | 2 | 0.017 |
| | broad PI | saquinavir | 3 | <0.001 |
| 73 | secondary | indinavir | 4 | 0.002 |
| | broad PI | saquinavir | 10 | <0.001 |
| V77I | secondary | indinavir | 2 | 0.026 |
| | broad PI | | | |
| V82A/T/F | indinavir | indinavir | 3 | 0.01 |
| | ritonavir | ritonavir | 2 | 0.03 |
| I84V | indinavir | indinavir | 6 | <0.001 |
| N88S | nelfinavir | nelfinavir | 11 | <0.001 |
| L90M | saquinavir | saquinavir | 2 | 0.012 |
| | nelfinavir | nelfinavir | 9 | <0.001 |

TABLE 4
Characteristic HLA-specific amino acid substitutions in HIV-1 RT for
those HLA alleles with strongest associations in models. %-percentage of
individuals of HLA type that have the substitution in their viral sequence.

| | Site(s) of allele | CTL epitope | Most common |
|---|---|---|---|
| | associated | (if known) | amino acid |
| | polymorphism | containing/flanking | substitution(s) |
| HLA allele | in HIV-1 RT | polymorphism | (%) |
| A2 | 39 | 32-41 | T39 |
| A11 | 53 | | E53 |
| | 166 | 158-166 LAI | K166 |
| | | L. Menendez-Arias, A. Mas, E. Domingo, Viral Immunol 11, | |
| | | 167-181 (1998). Q. J. Zhang. R. Gavioli. G. Klein. M. G. Masuccl, | |
| | | Proc Natl.Acad.Sci U.S.A 90. 2217-2221 (1993). | |
| | | L. Wagner et al., Nature 391, 908-911 (1998). S. C. Threlkeld | |
| | | et al., J Immunol 159, 1648-1657 (1997). | |
| A28 | 32 | | K32 |
| B5 | 135 | 128-135 IIIB | I135T/V |
| | | L. Menendez-Arias, A. Mas, E. Domingo, Viral Immunol 11, | reduced HLA |
| | | 167-181 (1998). N. V. Sipsas et at., J Clin Invest 99, 752-762 | binding in-vitro |
| | | (1997). H. Tomiyama et al., Hum Immunol 60, 177-186 | shown |
| | | (1999). | |
| B7 | 158 | 156-165 | A158 |
| | 165 | C. M. Hay et al., J Virol 73, 5509-5519 (1999). L. Menendaz-Arias, | T165 |
| | 169 | A. Mas, E. Domingo, Viral Immunol 11, | E169 |
| | | 167-181 (1998). C. Brander and B. D. Walker, in HIV | |
| | | molecular immunology database. B. T. M. Korber et al., | |
| | | Eds, New Mexico, 1997). | |
| B8 | 32 | 20-26 | K32 |
| B12 | 203 | 203-212 | E203 |
| | 211 | (HLA-B44) | R211 |
| B15 | 207 | | Q207 |
| B17 | 214 | | F214 |
| B18 | 68 | | S68 |
| | 135 | | I135 |
| | 138 | | E138 |
| | 142 | | I142 |
| B35 | 121 | 118-127 | D121 |
| | 177 | 175-185 | D177 |
| | | H. Shiga et al., AIDS 10, 1075-1083 (1996). | |
| B37 | 200 | | T200 |
| B40 | 197 | 192-201 | Q197 |
| | | (HLA-B60) | |
| | 207 | 207-216 | Q207 |
| | | (HLA-B60) | |

HIV-1 RT

All of the 63 polymorphisms positively (OR>1) associated with specific HLA-A or HLA-B allele(s) in these models (p≦0.05 in all cases) were plotted on a map of HIV-1 RT in relation to the overall rate of polymorphism at each residue and known CTL epitopes (FIG. 2). For 16 of these HLA-specific polymorphisms associations, the polymorphisms were located within or flanking CTL epitopes with corresponding HLA restriction, in keeping with CTL escape mutation and there appeared to be clustering of 14 associations along the sequence. HLA-associated polymorphisms were evident at four primary and nine non-primary anchor positions within the CTL epitopes and three were flanking CTL epitopes with corresponding HLA restriction. The characteristic amino acid substitutions present in those with the HLA alleles that had the strongest associations were then determined (Table 4). There were 32 negative HLA associations (OR<1) also evident-indicating that polymorphism, or change away from consensus was significantly less likely in the presence of these HLA alleles versus all others.

HIV-1 Protease

There were 48 HLA allele-specific polymorphisms in HIV-1 protease detected by the models (FIG. 4). There were clustered polymorphisms for 8 HLA alleles, including those associated with HLA-B5 at positions 12, 13, 14 and 16. There were HLA associated polymorphisms within and flanking the only two published CTL epitopes, though none corresponded to the predicted HLA restriction of the epitopes (based on binding motifs). The strongest HLA associations and their characteristic amino acid substitutions present in the cohort are shown in Table 5. There were 23 negative HLA associations detected.

TABLE 5
Characteristic HLA-specific amino acid substitutions in HIV-1 protease
for those HLA alleles with strongest associations in models.

| | Site(s) of allele associated | |
|---|---|---|
| | Polymorphism in HIV-1 | Most common amino acid |
| HLA allele | protease | substitution (%) |
| B5 | 12 | S (19.7%) |
| B7 | 10 | I (16.2%) |
| B12 | 35 | D (67.5%) |
| | 37 | S (27.9%) |
| B13 | 62 | V (9.5%) |
| B15 | 46 | I (7.5%) |
| | 90 | M (8.0%) |
| | 93 | L (51.6%) |
| B37 | 35 | D (54.6%) |
| | 37 | D (57.3%) |
| B40 | 13 | V (22.4%) |

Interactions Between Host HLA and Antiretroviral Drug Resistance Mutation

There were four antiretroviral drug resistance mutations in HIV-1 RT (M41L, K70R, T210W and T215Y/F) and seven in protease (L10I/R, M46I/L, A71V/T, 73, V77I, V82A/T/F and L90M) at which HLA alleles independently increased the probability of the mutation (FIGS. 2 and 4, Box B). For example, the odds of developing M41L were markedly increased in individuals carrying HLA-A28 compared with all other HLA-A or -B alleles (OR=41, p<0.001). To examine this observation in more detail, we analysed all individuals in the total cohort who had zidovudine exposure and HIV-1 RT sequencing at any time after treatment (n=265). The prevalence of HLA-A28 in this set of individuals (8.0%) was comparable to that of the total cohort (8.3%). However, the HLA-A28 allele was over-represented in the 58 zidovudine treated individuals with M41L (12.1%) compared with those 207 individuals who did not develop this substitution (7.7%, RR=1.69, p=0.30, Fisher's exact test). A similar analysis was carried out on all individuals who had nelfinavir treatment and HIV-1 protease sequencing (n=133). The presence of HLA-B13, associated with L90M in the logistic regression model (OR=13, p<0.001, FIG. 4), was present in 40.0% of individuals with L90M compared with 18.7% without L90M after taking nelfinavir (RR=2.96, p=0.12, Fisher's exact test).

HLA alleles reduced the odds of two primary RT inhibitor resistance polymorphisms, K103N (HLA-A19, 1/OR=4, p=0.04) and M184V (HLA-B16, 1/OR=4, p=0.03) and one secondary PI resistance mutation L10I/R/V (HLA-A10, 1/OR=4, p=0.024)(FIGS. 2 and 4, Box C), raising the possibility of antagonistic selection pressures in individuals with these specific HLA alleles treated with drugs that induce these mutations.

Discussion

The findings of this study support a highly dynamic, host-specific model of HIV-1 adaptation in-vivo, in which host CTL responses and antiretroviral therapy act as continuous, competing or parallel interacting evolutionary forces at the level of single viral residues.

The distribution of common, known drug resistance mutations in the study cohort were comparable to that found in other large and small observational studies, including those in drug naïve individuals. Almost all known primary and most secondary drug resistance mutations were evident as drug-associated polymorphisms across the population and in all these cases, the drug association corresponded to the known causative antiretroviral agents. The expected associations between D30N and nelfinavir and G48V and saquinavir were not detected, though there was (at least 30%) power to detect significant drug associations with OR>2 for both mutations. Notably, G48V has been reported most frequently in-vivo in patients taking high dose saquinavir monotherapy, which has almost never been used in this study cohort. In most cases, saquinavir has been used together with ritonavir. Failure to detect known drug-associated polymorphisms using a population-based approach may be due to a lack of statistical power if use of the drug or virological failure on the drug is rare in the population, or if the mutation is predominantly selected in-vitro but not in-vivo. This method may prove useful for future novel antiretroviral drugs as a systematic way to characterise the most frequent, in-vivo drug resistance mutations induced by the drugs, even if the putative resistance sites in-vitro are not known.

In the same models that confirmed the expected selection effects of antiretroviral drugs, sequence diversity of several viral residues across the population was substantially influenced by the HLA characteristics of individual hosts. Previously, several HLA allele-specific polymorphisms in HIV-1 RT have been shown to correspond to known or likely sites of CTL escape, be more specific for fine HLA subtypes compared with broad serotypes, increase in frequency over time and predict higher plasma viral load. The models of HIV-1 RT sequence diversity have been further refined in this study by the adjustment for drug induced changes, leaving a core set of 22 polymorphisms that we present as putative CTL escape mutations (Table 4). To date, CTL escape mutation in HIV-1 protease gene has not been proven experimentally and only two CTL epitopes are currently published. However, Protease (RPLVTIKI; positions 8 to 15) is a predicted CTL epitope based on the HLA-B5 binding motif and we found strong associations between HLA-B5 and a cluster of polymorphisms at positions 12, 13, 14 and 16 (FIG. 4). The considerable natural polymorphism of the protease gene has been noted in several studies and it is possible that at least some of this is CTL-driven (FIG. 4, Table 5). The selected polymorphisms in HIV-1 RT and protease shown in Tables 4 and 5 had one or all of the following key characteristics; their statistical association with a HLA allele was very strong and remained significant (p<0.05) after adjustment for drug associated changes, polymorphisms at other positions (i.e. possible secondary mutations) and/or multiple comparisons, they fell within known CTL epitopes with a corresponding HLA restriction or were clustered with other polymorphisms associated with the same HLA allele. In all cases, there was either one or two predominant amino acid substitution(s) in the individuals carrying the HLA allele and the allele-associated polymorphism, as would be expected for a functional mutation selected by the CTL response. In the case of I135T/V, this substitution has been shown by others to abrogate HLA binding to the viral epitope in-vitro. Thus, just as drug resistance mutations are considered ‘characteristic’ or signatures of exposure to a particular antiretroviral drug, these amino acid substitutions were characteristic for particular HLA alleles, and were evident in drug treated individuals.

Potent antiretroviral therapy with sustained suppression of HIV-1 replication has been shown to coincide with a diminution of anti-HIV CTL responses, suggesting that CTL escape is less likely to occur. The studies that have documented CTL escape to fixation over time in individuals have all been in the untreated. In this study cohort, individuals were more likely to have HIV-1 RT and/or protease sequencing performed during virological failure, rather than when successfully virologically controlled. Though we cannot determine the time at which each HLA-specific polymorphism typically first appears, the demonstration of independent HLA and drug associated effects on viral sequence implies that CTL may still exert selection pressure during or after a period of antiretroviral drug therapy in some individuals.

There are a few viral residues where CTL pressure and drug pressure appeared to compete or concur in driving to either change or not change from the wildtype amino acid. This raises the intriguing possibility that anti-HIV CTL responses could be an explanation for discordance of in-vitro/in-vivo drug resistance patterns, discordance of genotypic and phenotypic resistance and variable rates of emergence of drug resistance mutations in different individuals. Interactions between CTL pressure and drug pressure are therefore germane to many aspects of contemporary treatment strategy, such as comparisons of different antiretroviral regimens, structured treatment interruptions (STIs) and different timing of treatment initiation. It is increasingly acknowledged that the design and interpretation of studies on these issues is limited by an incomplete understanding of what determines biological variability in disease between individuals. Our findings to date argue for HLA typing and viral genotyping to inform the design of future clinical studies. For example, STIs would not be expected to enhance HIV specific CTL responses in individuals who have already escaped from those responses in-vivo. Being able to prospectively identify individuals with or without the key escape mutations for their HLA, would enable STIs to be administered to those most likely to benefit from them. Similarly, studies of individualised drug choice and treatment timing could be informed by this data. In the same way that baseline and periodic post-treatment RT and protease resistance genotyping has now become the standard of care for optimisation of drug treatment, viral genotyping for critical escape mutations may greatly enhance individualisation of antiretroviral treatment in the future.

### EXAMPLE 3

Evidence of HIV-1 Adaptation to HLA-Restricted Immune Responses at a Population Level

Polymorphism Rate and Functional Constraint in HIV-1 RT

The relationship between polymorphism rate at single residues in HIV-1 RT and the known functional characteristics of the residues was examined (1). The polymorphism rates at the critical catalytic residues in HIV-1 RT (n=3, 0.53%), stability residues (n=37, 1.06%) and functional residues (n=11, 3.05%) were lower than at external residues (n=10, 5.95%) (P=0.0009, Wilcoxon).

Statistical methods Power calculations, covariate selection procedures and randomisation procedures are described in detail below.

Steps in the analysis at a single amino acid—an example using position 135 of HIV-1 RT

Any substitution of population sequence consensus amino acid (isoleucine) at position 135 of HIV-1 RT, ie I135x was set as the outcome/response variable. The starting covariates/explanatory variables were all HLA-A and -B alleles present in all individuals (n=473): A1, A2, A3, A9, A10, A11, A19, A28, A31, A36, B5, B7, B8, B12, B13, B14, B15, B16, B17, B18, B21, B22, B27, B35, B37, B40, B41, B42, B55, B56, B58, B60, B61. Serologically defined broad alleles were considered, rather than subtypes defined by high resolution DNA sequence based typing, so that data on all individuals in the cohort could be included. Furthermore, for several published CTL epitopes in HIV-1 RT, the HLA restriction of the epitope to the level of high resolution typing is not known.

Step 1—Power Calculations

Formal power calculations effectively exclude at the outset any HLA allele/position combinations for which there is insufficient statistical power (because of rarity of polymorphism, rarity of HLA allele or both) to be realistically examined for association. This considerably restricts the number of covariates and therefore the number of comparisons made within models. Power calculations also formally identify which HLA associations cannot be excluded by our analysis and would need examination in a larger dataset. Standard formulae are used for power calculations (2). The numbers of patients with each HLA allele and with I135x are used to calculate the power to detect an association with an odds ratio (OR) of 2 (positive association) or 0.5 (negative association). HLA alleles with less than 30% power are removed. The removed alleles at position 135 are A31, A36, B42, B55, B56, B58 and B61. It is important to note that we had less power to detect negative associations than positive associations. For example, at the mean HLA frequency of 10.9 and mean polymorphism rate of 4.0%, we had 30% power to detect an OR of 2.0 (ie a positive association) but only 5.6% power to detect an equivalent negative OR of 0.5.

Step 2

The numbers of individuals with and without each HLA allele, and with and without I135x are calculated. In order to remove covariates that may lead to an unstable logistic regression model, HLA alleles are eliminated if there are fewer than five individuals in any of the comparison groups. The removed alleles at position 135 are HLA-B37, B41 and B60.

Step 3

Covariates were then assessed separately for association with I135x using Fisher's exact test, and only those with univariate P-values≦0.1 are included in further analyses. The removed alleles are A1, A2, A3, A9, A11, A19, A28, B7, B8, B13, B14, B15, B16, B21, B22, B27 and B35.

Step 4—Forward Selection

If the number of covariates remaining exceeds 10% of the number of individuals, forward selection using logistic regression is used to choose the covariates that are to remain in the analysis. Covariates are selected sequentially based on the smallest P-value for an added covariate until the number equals 10% of the number of patients. At position 135, the number of covariates was less than 10% of the number of patients so no selection was needed.

Step 5—Backwards Elimination

A standard backwards elimination procedure is then carried out. Logistic regression models are fitted for the remaining covariates. If any of the P-values for the covariates is greater then 0.1, after accounting for the other included covariates, then the covariate with the largest P-value is removed and the logistic model refitted. This is repeated until all covariates have a P-value less than 0.1. At position 135, this removes HLA alleles B12, B17 and B40.

Step 6—Exact P-Values

To accommodate relatively small samples, “exact” P-values are based on randomisation tests rather than the usual large sample approximations (3). In this procedure, the final covariate sets are randomly permuted amongst individuals and the standard test statistics for association with I135x calculated for each permutation. 1000 random permutations are generated for each model and the P-value is based on the appropriate percentage of test values more extreme than that pertaining to the actual data. The proportion of times that a covariate has a test statistic in the random datasets exceeding that from the actual data is calculated for each covariate. This proportion gives a randomisation (exact) P-value. Covariates with exact P-values greater than 0.05 are removed sequentially and those with P-values less than 0.05 are considered significant. At position 135, this removes the alleles HLA-A10 and -B18, leaving HLA-B5 as the significant association with I135x.

Correction for Multiple Comparisons

In order to highlight the significant HLA associations whose P-values withstand correction for the number of comparisons made across the whole analysis (ie a very low P-value cut-off for higher specificity but lower sensitivity), correction factors were generated for each HLA allele. Positive and negative associations were considered separately. 1000 randomised datasets were created from the original dataset as described above. The entire selection process including the preliminary model reduction procedures was then carried out for each amino acid residue and the total number of significant associations for each HLA allele across all positions was calculated. For example, for HLA-A2 there were, on average, 1.827 positive HLA-A2 associations across all residues per random dataset. This number was divided by 0.05 to a multiple comparisons correction factor (x) for HLA-A2. This correction factor is the estimated equivalent number of “independent” tests carried out. The correction factor was applied to the P-values calculated in the actual data using Bonferroni adjustment [i.e. p*=1−(1−p)^{x}, where p is the P-value from the model using the actual data, x is the correction factor and p* is the corrected P-value].

Overall P-Value for Actual Vs Randomised Data

The overall P-value for all associations at all positions was obtained by considering the extremeness of the sum of the individual tests at each position relative to the values of this sum obtained from the randomisation data sets. The sum of all test statistics for all models for all alleles using the actual data was calculated. The same was done for the randomised datasets. For none of the 1000 random datasets was this number greater than the actual data, giving an overall P-value of < 1/1000 or <0.001.

Significance of Associations within ‘Known’ CTL Epitopes

We conducted analyses to determine the probability of finding by chance at least 15 significant positive associations within ‘corresponding’ known CTL epitopes (ie restricted to the same HLA allele). If significant HLA associations were occurring randomly across residues, the probability that an HLA association would occur within the known CTL epitope restricted to that allele equates to the relative proportion of all residues falling within the epitope. The total number of significant associations within known epitopes is then a sum of non-identical binomial variables, whose distribution can be evaluated via simulation, for example. Only 4.27 significant positive associations within known epitopes were expected based on the random hypothesis compared with the 15 observed. The approximate P-value for this is <0.001.

### EXAMPLE 4

### Confirmation of the Identification of CTL Epitopes

Using the methods described herein the inventors have been able to identify various CTL epitopes. Since the filing of the provisional application and the filing of the complete application other groups have independently reported a number of these epitopes e.g An HLA-A11 restricted CTL epitope has been described between positions 117 and 126 of HIV reverse transcriptase (B. Sriwanthana et al., Hum Retroviruses 17, 719-34 (2001)). The provisional application identified an HLA-A11 association at position 122 of HIV reverse transcriptase. Also the following associations were also identified within subsequently published CTL epitopes: HLA-A3 at 101 within an HLA-A3 restricted CTL epitope RT (93-101; C. Brander and P. Goulder, in HIV Molecular Immunology Database. B. T. M. Korber et al., Eds. New Mexico, 2001); HLA-A19(30) at 178 within an HLA-A*3002 epitope (173-181; C. Brander and P. Goulder, in HIV Molecular Immunology Database. B. T. M. Korber et al., Eds. New Mexico, 2001; and P. Goulder et al., J. Virol 75(3), 133947 (2001)) and HLA-B40 at 207 within an HLA-B*4001 restricted CTL epitope (202-210; C. Brander and P. Goulder, in HIV Molecular Immunology Database. B. T. M. Korber et al., Eds. New Mexico, 2001).

### EXAMPLE 5

### Therapeutic Development

HIV and ancestral retroviruses have evolved under intense selective pressure from HLA (or MHC) restricted immune responses. HIV has highly dynamic and error prone replication and evidence of this HLA restricted selective pressure can be seen in individual patients and at a population level. Of 473 Western Australian patients studied, no two patients had the same HIV Reverse Transcriptase amino acid sequence. Polymorphisms were most evident at sites of least functional or structural constraint and frequently were associated with particular host HLA Class I alleles. Patients who had escape mutations at these HLA-associated viral polymorphisms had a higher HIV viral load. This information indicates which HIV peptides (epitopes) stimulate the strongest protective immune response against the virus after infection. Those same epitopes should afford the strongest protection if given in a vaccine before exposure to the virus.

The protection afforded by a preventative HIV vaccine will depend on the breadth and strength of the HLA restricted immune responses elicited by the therapeutic and the extent to which the infecting HIV sequence has escaped those responses. The objective is (1.) for the therapeutic to induce the maximum number and strength of HLA-restricted CTL responses and (2.) to have the maximum number of identical matches between therapeutic epitopes and incoming viral epitopes (or for the viral epitopes to at least be similar enough to the therapeutic epitope to still be recognized by the therapeutic induced CTL response).

The traditional approach has been to try to include conserved epitopes—stretches of viral proteins that are eight to 12 amino acids long that are invariably present in all HIV variants. However, studies presented herein indicate that the virus and its ancestors have evolved under intense selective pressure from HLA-restricted immune responses and therefore tend not to have conserved epitopes recognized by common HLA types.

A preliminary analysis of the first 80 patients with full-length sequencing has revealed HLA specific associations in all the proteins and escape at these residues correlated with a higher pre-treatment viral load. The strongest associations and their relationship to HIV viral load are shown in Table 6. FIG. 5 shows the relationship between the degree of viral adaptation to HLA-restricted responses and the viral load. The number and strength of HLA-restricted associations and the degree to which these explain the variability in pre-treatment viral load will increase as data on a larger number of patients becomes available.

TABLE 6

| | Amino | | | | Estimated | | |
|---|---|---|---|---|---|---|---|
| | acid | | Odds | | change in | Consensus | Non/escaped |
| Protein | position | HLA | ratio | P-value | viral load | amino acid | amino acid |
| Integrase | 11 | B*4402 | 166.02 | <0.0001 | 1.39 | Glutamate | Aspartate |
| Nef | 14 | C*0701 | 6.78 | 0.0001 | 0.31 | Proline | Serine |
| p6 | 34 | A*2402 | 52.59 | 0.0002 | −0.02 | Glutamate | Aspartate |
| Nef | 71 | B*0702 | 19.40 | 0.0002 | 0.28 | Arginine | Lysine |
| p6 | 25 | B*4402 | 66.34 | 0.0003 | 0.91 | Serine | Proline |
| Integrase | 119 | DRB1- | 429.45 | 0.0004 | −1.10 | Serine | Arginine |
| | | 0101 | | | | | |
| Vpr | 84 | DRB1- | 0.03 | 0.0005 | −0.45 | Threonine | Isoleucine |
| | | 0701 | | | | | |
| Integrase | 122 | C*0501 | 17.24 | 0.0005 | 0.63 | Threonine | Isoleucine |
| Integrase | 119 | DRB1- | 144.67 | 0.0005 | −0.12 | Serine | Glycine |
| | | 0701 | | | | | |
| Protease | 37 | DRB1- | 19.98 | 0.0006 | 0.23 | Asparagine | Serine |
| | | 1302 | | | | | |
| Integrase | 17 | B*4001 | 8.00 | 0.0008 | −0.31 | Serine | Asparagine |
| p6 | 29 | A*2402 | 9.38 | 0.0008 | 0.43 | Glutamate | Glycine |
| Integrase | 119 | B*4402 | 273.63 | 0.0009 | 0.53 | Serine | Proline |
| p7 | 9 | B*1801 | 30.54 | 0.0010 | 0.20 | Glutamine | Proline |

FIG. 5 shows the relationship between the degree of viral adaptation to HLA-restricted responses and the viral load.

A simulation was undertaken to determine the likely efficacy of different preventative vaccine candidates assuming an HIV negative target population with the same HLA diversity as the HIV positive Western Australian cohort was exposed to the same range of viral diversity observed in the Western Australian HIV positive cohort. In other words a hypothetical population of 249 HIV negative patients with the identical HLA types as the 249 HIV positive Western Australian patients was examined. The possibility of the first HIV negative patient being exposed to the virus sequenced in the first HIV infected patient was considered, then the virus in the second HIV positive patient and so on until all 80 viral sequences had been considered. This process was repeated for the second hypothetical HIV negative patient and so on until all 249 HIV negative subjects had been considered.

In the first analysis shown on FIG. 6 the inventors calculated for each potential therapeutic candidate how many beneficial amino acid residues were present in the therapeutic (i.e. a consensus at a positive HLA association and a match between the therapeutic and the incoming virus, or second most common residue at a negative HLA association and a match between this second most common residue and the incoming virus). The optimized vaccination sequence shown below used the population consensus at all residues except those with predominant negative HLA associations in which case the second most common residue in the population was used.

The optimal therapeutic sequence: (Genes are underlined. Proteins for which these genes encode are in italics. Gag, pol and envelope encode several proteins. Other genes encode just one protein with the same name as the gene.)
(i) Gag (p17, p24, p2, p7, p1, p6) (SEQ ID NO: 2)

Having regard to the foregoing analysis the following Gag (p17, p24, p2, p7, p1, p6) amino acid sequence has been elucidated which is expected to provide optimal CTL induced therapeutic protection to the cohort examined in the present studies:

(SEQ ID NO: 3)

(ii) Pol (integrase, reverse transcriptase,
integrase)
MGARASVLSGGELDRWEKIRLRPGGKKKYKLKHIVWASRELERFAVNPGL
LETSEGCRQILGQLQPSLQTGSEELKSLYNTVATLYCVHQRIEVKDTKEA
LDKIEEEQNKSKKKAQQAAADTGNSSQVSQNYPIVQNLQGQMVHQAISPR
TLNAWVKVVEEKAFSPEVIPMFSALSEGATPQDLNTMLNTVGGHQAAMQM
LKETINEEAAEWDRLHPVHAGPIAPGQMREPRGSDIAGTTSTLQEQIGWM
TNNPPIPVGEIYKRWIILGLNKIVRMYSPTSILDIRQGPKEPFRDYVDRF
YKTLRAEQASQEVKNWMTETLLVQNANPDCKTILKALGPAATLEEMMTAC
QGVGGPGHKARVLAEAMSQVTNSATIMMQRGNFRNQRKTVKCFNCGKEGH
IARNCRAPRKKGCWKCGKEGHQMKDCTERQANFLGKIWPSHKGRPGNFLQ
SRPEPTAPPEESFRFGEETTTPSQKQEPIDKELYPLASLRSLFGNDPSSQ

Having regard to the foregoing analysis the following Pol (integrase, reverse transcriptase, integrase) amino acid sequence has been elucidated which is expected to provide optimal CTL induced therapeutic protection to the cohort examined in the present studies:

(SEQ ID NO: 4)

(iii) vif
FFRENLAFPQGKAREFSSEQTRANSPTRRELQVWGEDNNSTSEAGADRQG
TVSFSFPQITLWQRPLVTIKIGGQLKEALLDTGADDTVLEEMNLPGRWKP
KMIGGIGGFIKVRQYDQIIIEICGHKAIGTVLVGPTPVNIIGRNLLTQLG
CTLNFPISPIETVPVKLKPGMDGPKVKQWPLTEEKIKALVEICTEMEKEG
KISKIGPENPYNTPVFAIKKKDSTKWRKLVDFRELNKRTQDFWEVQLGIP
HPAGLKKKKSVTVLDVGDAYFSVPLDKDFRKYTAFTIPSINNETPGIRYQ
YNVLPQGWKGSPAIFQSSMTKILEPFRKQNPDIVIYQYMDDLYVGSDLEI
GQHRTKIEELRQHLLKWGFTTPDKKHQKEPPFLWMGYELHPDKWTVQPIV
LPEKDSWTVNDIQKLVGKLNWASQIYAGIKVRQLCKLLRGTKALTEVIPL
TEEAELELAENREILKEPVHGVYYDPSKDLIAEIQKQGQGQWTYQIYQEP
FKNLKTGKYARMRGAHTNDVKQLTEAVQKIATESIVIWGKTPKFKLPIQK
ETWEAWWTEYWQATWIPEWEFVNTPPLVKLWYQLEKEPIVGAETFYVDGA
ANRETKLGKAGYVTDRGRQKVVSLTDTTNQKTELQAIHLALQDSGLEVNI
VTDSQYALGIIQAQPDKSESELVSQIIEQLIKKEKVYLAWVPAHKGIGGN
EQVDKLVSAGIRKVLFLDGIDKAQEEHEKYHSNWRAMASDFNLPPVVAKE
IVASCDKCQLKGEAMHGQVDCSPGIWQLDCTHLEGKIILVAVHVASGYIE
AEVIPAETGQETAYFLLKLAGRWPVKTIHTDNGSNFTSTTVKAACWWAGI
KQEFGIPYNPQSQGVVESMNKELKKIIGQVRDQAEHLKTAVQMAVFIHNF
KRKGGIGGYSAGERIVDIIATDIQTKELQKQITKIQNFRVYYRDSRDPLW
KGPAKLLWKGEGAVVIQDNSDIKVVPRRKAKIIRDYGKQMAGDDCVASRQ
DED

Having regard to the foregoing analysis the following vif amino acid sequence has been elucidated which is expected to provide optimal CTL induced therapeutic protection to the cohort examined in the present studies:

(SEQ ID NO: 5)

(iv) vpr
MENRWQVMIVWQVDRMRIRTWKSLVKHHMYISKKAKGWFYRHHYESTHPR
ISSEVHIPLGDAKLVITTYWGLHTGERDWHLGQGVSIEWRKRRYSTQVDP
DLADQLIHLYYFDCFSESAIRNAILGHIVSPRCEYQAGHNKVGSLQYLAL
AALITPKKIKPPLPSVTKLTEDRWNKPQKTKGHRGSHTMNGH

Having regard to the foregoing analysis the following vpr amino acid sequence has been elucidated which is expected to provide optimal CTL induced therapeutic protection to the cohort examined in the present studies:

(SEQ ID NO: 6)

(v) tat
MEQAPEDQGPQREPYNEWTLELLEELKSEAVRHFPRIWLHGLGQHIYETY
GDTWAGVEAIIRILQQLLFIHFRIGCQHSRIGITRQRRARNGASRS

Having regard to the foregoing analysis the following tat amino acid sequence has been elucidated which is expected to provide optimal CTL induced therapeutic protection to the cohort examined in the present studies:

(SEQ ID NO: 7)

(vi) rev
MEPVDPRLEPWKHPGSQPKTACTNCYCKKCCFHCQVCFIKKGLGISYGRK
KRRQRRRAPQDSQTHQVSLSKQPASQPRGDPTGPKESKKKVERETETDPV
D

Having regard to the foregoing analysis the following rev amino acid sequence has been elucidated which is expected to provide optimal CTL induced therapeutic protection to the cohort examined in the present studies:

(SEQ ID NO: 8)

(vii) Vpu
MAGRSGDSDEELLKTVRLIKFLYQSNPPPSPEGTRQARRNRRRRWRERQR
QIRSISGWILSTYLGRPAEPVPLQLPPLERLTLDCNEDCGTSGTQGVGSP
QILVESPAVLESGTKE*

Having regard to the foregoing analysis the following vpu amino acid sequence has been elucidated which is expected to provide optimal CTL induced therapeutic protection to the cohort examined in the present studies:

(SEQ ID NO:9)

(viii) envelope (gp120, gp41)
MQPLEILAIVALVVAAIIAIVVWTIVFIEYRKILRQRKIDRLIDRIRERA
EDSGNESEGEESALVEMGVEMGHHAPWDVDDL

Having regard to the foregoing analysis the following envelope (gp120, gp41) amino acid sequence has been elucidated which is expected to provide optimal CTL induced therapeutic protection to the cohort examined in the present studies:

(SEQ ID NO: 10)

(ix) nef
MRVKGNNQHLWKWGWKWGTMLLGMLMICSATEKLWVTVYYGVPVWKEATT
TLFCASDAKAYDTEVHNVWATHACVPTDPNPQEVVLENVTENFNMWKNNM
VEQMHEDIISLWDQSLKPCVKLTPLCVTLNCTDLNNDTNTNNTSGSNNME
KGEIKNCSFNITTSIRDKMQKEYALFYKLDVVPIDNDNTSYRLISCNTSV
ITQACPKVSFEPIPIHYCAPAGFAILKCNDKKFNGTGPCTNVSTVQCTHG
IRPVVSTQLLLNGSLAEEEVVIRSENFTNNAKTIIVQLNESVEINCTRPN
NNTRKSISIHIGPGRAFYATGEIGDIRQAHCNISRAEWNNTLKQIVKKLR
EQFGKNKTIVFNQSSGGDPEIVMHSFNCGGEFFYCNTTQLFNSTWNNSTW
NTEESNNTEGNETITLPCRIKQIINMWQEVGKAMYAPPIRGQIRCSSNIT
GLLLTRDGGNNNNKTETFRPGGGDMRDNWRSELYKYKWKIEPLGVAPTKA
KRRVVQREKRAVGIGAMFLGFLGAAGSTMGAASITLTVQARQLLSGIVQQ
QNNLLRAIEAQQHLLQLTVWGIKQLQARVLAVERYLKDQQLLGIWGCSGK
LICTTAVPWNTSWSNKSLNKIWDNMTWMEWEKEINNYTGIIYNLIEESQN
QQEKNEQELLELDKWASLWNWFDISKWLWYIKIFIMIVGGLIGLRIVFAV
LSIVNRVRQGYSPLSFQTHLPTPRGPDRPEGIEEEGGERDRDRSSRLVDG
FLAIIWDDLRSLCLFSYHRLRDLLLIVTRIVELLGRRGWEILKYWWNLLQ
YWSQELKNSAVSLLNATAIAVAEGTDRIIEVVQRACRAILHIPRRIRQGV
ERALL

Having regard to the foregoing analysis the following nef amino acid sequence has been elucidated which is expected to provide optimal CTL induced therapeutic protection to the cohort examined in the present studies:

MGGKWSKSSMVGWPAVRERMRRAEPAADGVGAVSRDLEKHGAITSSNTAA
TNADCAWLEAQEEEEVGFPVRPQVPLRPMTYKGALDLSFFLKEKGGLEGL
IYSQKRQDILDLWVYHTQGYFPDWQNYTPGPGIRYPLTFGWCFKLVPVEP
EKVEEANEGENNSLLHPMSQHGMDDPEREVLMWKFDSRLAFRHMARELHP
EYYKDC

In the second analysis shown in FIG. 6 an estimated strength of the HLA-restricted immune response was calculated that would be induced by each therapeutic in response to each of the potential incoming viruses using the viral load results as illustrated in the estimated change in viral load column shown in Table 6.

Generally the use of consensus sequence for the study population reduced but did not eliminate the problem posed by the viral diversity and inclusion of the maximum number of HLA-A, B or C specific viral polymorphisms (particularly those associated with large viral load increases on escape) is predicted to improve HLA-restricted responses.

Therapeutic design can be undertaken as demonstrated here in the Western Australian population using whole length sequencing to determine the optimal parts of the virus to include in the therapeutic. Once the therapeutic has been designed these analyses can be repeated in the target population for vaccination (e.g. the a U.S., African or European population) but this time only the part of the virus included in the therapeutic need be sequenced in the target population to estimate efficacy of the vaccine in that population (i.e. with different viral and HLA diversity)

### EXAMPLE 6

### Preparation of Therapeutics

Employing the above mentioned modelling to estimate the therapeutic efficacy of a potential vaccine candidate in a particular target population a single optimal amino acid sequence for the target HIV infected Western Australian population was determined. In this case the HLA type and challenge virus is known for each patient and one therefore only considers the HIV infected population and can optimise the number of non-escaped HLA-specific residues in the therapeutic (i.e. consensus at positive associations and second most common residue at negative associations). From the use of these techniques the above mentioned sequences (ie proteins Gag (p17, p24, p2, p7, p1, p6) (SEQ ID NO:2), Pol (integrase, reverse transcriptse, integrase) (SEQ ID NO: 3), vif (SEQ ID NO: 4), vpr (SEQ ID NO: 5), tat (SEQ ID NO: 6), rev (SEQ ID NO: 7), vpu (SEQ ID NO: 8), envelope (gp120, gp41) (SEQ ID NO: 9), and nef (SEQ ID NO: 10)) were selected in the prevention of HIV infection in this and like populations.

1. A therapeutic to treat HIV specific immune responses

At the commencement of treatment a blood sample is taken from each patient for use in HIV sequencing and HLA typing to determine which residues and hence virus populations have already escaped from HLA-restricted immune response using the HLA-viral polymorphism associations derived from our population based analysis. The methods for carrying out this analysis are described above.

Although vaccination is best individualized to those residues and hence virus populations that have not yet escaped, for a single population based vaccine, a vaccine optimized with consensus residues at positive associations at pre-treatment sequences and the second most common residue at residues with predominant negative associations to common alleles is used. According to this example, the patient is vaccinated by a process of introducing one or more vectors into the patient, which are adapted to express the optimized protein sequence of the vaccine. While the vector may express all of the proteins Gag (p17, p24, p2, p7, p1, p6) (SEQ ID NO:2), Pol (integrase, reverse transcriptse, integrase) (SEQ ID NO: 3), vif (SEQ ID NO: 4), vpr (SEQ ID NO: 5), tat (SEQ ID NO: 6), rev (SEQ ID NO: 7), vpu (SEQ ID NO: 8), envelope (gp120, gp41) (SEQ ID NO: 9), and nef (SEQ ID NO: 10), preferentially the vaccine only comprising the proteins: Gag (p17, p24, p2, p7, p1, p6) (SEQ ID NO: 2), Pol (integrase, reverse transcriptse, integrase) (SEQ ID NO: 3), and nef (SEQ ID NO: 10).

Delivery of the vaccine to the patient is achieved using a fowlpox vector (or any other vector suitable for deliver of a protein sequence to a patient). This is achieved by well known and standard techniques which include isolation of a nucleotide sequence that encodes the proteins that are used in the vaccine. The nucleotide sequence is then inserted into the vector (eg fowlpox) and then delivered to a patient at levels and in a manner that leads to protein expression within the patient.

If the HIV sequence selected for use in the vaccine does not encode the specific sequence mentioned that sequence may be modified using well known and well understood techniques in molecular biology (see Ausubel, F., Brent, R., Kingston, R. E., Moore, D. D., Seidman, J. G., Smith, J. A., Struhl, K. Current protocols in molecular biology. Greene Publishing Associates/Wiley Intersciences, New York., the text of which is incorporated herein by reference) including site directed mutagenesis techniques as an example.

2. A vaccine to maintain HIV specific immune responses as HIV antigen wanes during effective highly active antiretroviral therapy.

According to this methodology at the commencement of treatment a blood sample is taken from each patient for use in HIV sequencing and HLA typing to determine which residues and hence virus populations have already escaped from HLA-restricted immune response using the HLA-viral polymorphism associations derived from our population based analysis. The methods for carrying out this analysis are described above.

The patient is then placed on HMRT to inhibit HIV replication decreasing the availability of HIV antigen to sustain HIV antigen specific immune responses. The protocols in the HMRT treatment used depend on the patient to be treated. Physicians will adopt an appropriate protocol based on the level of infection in a patient, the health of the patient etc.

Over the course of HMRT therapy regular monitoring of viral loads is carried out to measure the effect of treatment. Once viral load has waned sufficiently the patient is then placed on a vaccination protocol in accordance with the previous example which leads to delivery of the fowlpox vectors to the patient, which encode one or more of the proteins employed in the optimized vaccine as identified by the above methodology. Desirably the therapeutic delivered to the patient will encode at least pol, gag and nef proteins as herein described, however it will be appreciated that the precise constitution of the therapeutic may vary depending on the precise needs of the treating physician.

3 A vaccine to prevent or delay the emergence of anti-retroviral drug resistance mutations in patients on highly active antiretroviral therapy.

Combination antiretroviral therapy (ART) has resulted in a 60% reduction in mortality from HIV-1 and provided great hope for those infected. However the development of drug resistance is a major hurdle in the long-term benefit it can provide both in the developed and developing world. Resistance to HIV medications following treatment is now common, with studies in the USA and Ivory Coast demonstrating over 50% of treated patients harbouring some resistance to HIV.

Vaccination aims to prevent the onset of disease states and has provided incalculable benefit to entire communities and humanity as a whole. The role of vaccination in those already infected with a particular disease is only currently being evaluated, especially in relation to HIV-1. A vaccine that could prevent or delay the development of drug resistance in those already infected with HIV-1 could provide significant benefit for the millions of people living with this disease.

The clinical benefit of therapeutic vaccines in HIV infected patients has been disappointing to date potentially because the patient has already been exposed to the vaccine antigens and the vaccines epitopes are to a variable extent escaped from HLA-restricted immune responses. Antiretroviral resistance mutations are detrimental to the patient but in this case the patient has not yet been exposed to the antigen. Use of a sufficiently immunogenic vaccine such as the DNA/Fowlpox prime/boost vaccine should provide high level T cell immunogenicity. A therapeutic vaccine has been designed using the following principles:
1. Encode common resistance mutations
2. Encode putative “fitness mutations” where these do not interfere with common key mutations
3. Use whole protein as much as possible but avoid long stretches of wild-type amino acids as response to wild type sequence is relatively undesirable
4. Use the optimised consensus-like sequence described in Example 1 as the backbone (i.e. the amino acid sequence at the residues that are not sites of anti-retroviral resistance mutation). Where possible (e.g. protease) use a backbone known to fold appropriately (e.g. a real isolate) as antigen stability may be better.
5. Where resistance mutations are close together (<4 amino acids) generate separate fragments expressing only a single resistant epitope, as responses to epitopes containing 2 resistance mutations are relatively undesirable
6. For fragments containing a single mutation, encode 7 amino acids on either side to enhance development of CD8 T cell response to encoded mutation and reduce likelihood of response to wild-type sequence
7. However, encode as few as possible separate fragments as responses to amino acids sequences which overlap 2 fragments (irrelevant epitopes) is undesirable
8. Separate fragments which contain same coding sequence as much as possible as lessens potential for recombination during construction Using these principles the following therapeutic sequences have been developed (as illustrated in FIGS. 7 and 8):
Protease vaccine: Having regard to the foregoing analysis the following protease amino acid sequence has been elucidated which is expected to provide optimal CTL induced therapeutic protection to the cohort examined in the present studies:

Optimal CTL and Drug Vaccine

(SEQ ID NO: 11)

PQITLWQRPIVTIKIGGQLREALLDTGADNTVLEEMNLPGRWKPKIIGGV
GGFIKVRQYDQIPIEICGHKAIGTVLVGPTPANIIGRNLMTQIGCTLNFG
RWKPKMIVGIGGLIKVRQYDQLVGPTPVNVIGRNLLTQ

Same Peptide with Population Consensus Amino Acids

(SEQ ID NO: 12)

PQITLWQRPLVTIKIGGQLKEALLDTGADDTVLEEMNLPGRWKPKMIGGI
GGFIKVRQYDQIPIEICGHKAIGTVLVGPTPVNIIGRNLLTQIGCTLNFG
RWKPKMIGGIGGFIKVRQYDQLVGPTPVNIIGRNLLTQ

RT vaccine: Having regard to the foregoing analysis the following RT amino acid sequence has been elucidated which is expected to provide optimal CTL induced therapeutic protection to the cohort examined in the present studies:

Optimal CTL and Drug Vaccine

(SEQ ID NO: 13)

LVEICTELEKEGKISTPVFAIKRKDSTRWRKLVDFDIVIYQYVDDLYVGS
HLLKWGFYTPDKKHQICTEMEKDGKISKIGAIKKKDSDKWRKVVDFRELN
QLGIPHPGGLKKNKSVTVLDVGDAYFSIPLDKDFRYQYNVLPMGWKGSPA
QNPDIVICQYMDDLYVASDLEIGQHRTKIEELRQHLWKWGFFTPDQKHQK
EPP

Same Peptide with Population Consensus Amino Acids

(SEQ ID NO: 14)

LVEICTEMEKEGKISTPVFAIKKKDSTKWRKLVDFDIVIYQYMDDLYVGS
HLLKWGFTTPDKKHQICTEMEKEGKISKIGAIKKKDSTKWRKLVDFRELN
QLGIPHPAGLKKKKSVTVLDVGDAYFSVPLDKDFRYQYNVLPQGWKGSPA
QNPDIVIYQYMDDLYVGSDLEIGQHRTKIEELRQHLLKWGFTTPDKKHQK
EPP

The objective is for the therapeutic construct to match the new epitope created when the anti-retroviral drug resistance mutation emerges.

Ideally the autologous virus in each patient would be sequenced and an identical virus in all respects apart from the introduction of characteristic drug mutations be used in the therapeutic construct (i.e. a vaccine individualized to each patient). However, such an approach would be labor intensive and impractical at this time (each vaccine has to be separately tested and licensed). The therapeutic modeling similar but not identical to approach described above could be used to determine a single optimal amino acid sequence for the target HIV infected Western Australian population. In this case the HLA type and challenge virus is known for each patient and we therefore only consider the HIV infected population and optimize the number of non-escaped HLA-specific residues in the vaccine (i.e consensus at positive associations and second most common residue at negative associations)

According to this example, the patient is vaccinated by a process of introducing one or more vectors into the patient, which are adapted to express the optimized protein sequence of the vaccine.

Reactions and manipulations involving nucleic acid techniques, unless stated otherwise, were performed as generally described in Sambrook et al., 1989, Molecular Cloning: A Laboratory Manual, Cold Spring Harbor Laboratory Press, and methodology.

A fowlpox vector is first constructed containing the cDNA encoding the protease and RT amino acid sequences mentioned above. Insertion of the cDNA sequence encoding the aforementioned amino acid sequences should be carried out in a manner to ensure that the sequences will be expressed when introduced into a patient. The vector may also contained all expression elements necessary to achieve the desired transcription of the sequences. Other beneficial characteristics can also be contained within the vectors such as mechanisms for recovery of the nucleic acids in a different form.

The constructed vector is then introduced into cells by any one of a variety of known methods within the art. Methods for transformation can be found in Sambrook et al., Molecular Cloning: A Laboratory Manual, Cold Springs Harbor Laboratory, New York (1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995) and Gilboa, et al. (1986) and include, for example, stable or transient transfection, lipofection, electroporation and infection with recombinant viral vectors.

### EXAMPLE 7

### Additional Specific Examples of Therapeutic Amino Acid Sequences Used in the Treatment of HIV Infection

Following the protocols set out in Example 1 and 2 the following amino acid sequences were revealed, which provide a means for specific treatment of HIV infected individuals, which have the specific HLA associations mentioned.

(SEQ ID NO: 15)

(i) FLDGIDKAQEEHEKYHSNWRAM
and HLA-B*4402

A change in amino acid residue from the consensus amino acid of glutamate (E) at position 11 of the protein integrase occurs more often than expected by chance in individuals with HLA-B*4402 than in patients without this HLA allele (odds ratio=166, P-value<0.0001; after adjustment for other HLA alleles). Furthermore, HLA-B*4402 positive individuals with an amino acid other than glutamate at position 11 of integrase have increased viral load compared to those HLA-B*4402 positive patients with a glutamate at this position. Hence a therapeutic that included the consensus amino acid of glutamate at position 11 would offer protection to HLA-B*4402 positive patients compared to the most common other amino acid seen in these patients at this position of an aspartate (D). Therefore, the amino acid sequence FLDGIDKAQEEHEKYHSNWRAM (SEQ ID NO: 15) should provide protection to HLA-B*4402 positive patients if included in a therapeutic while the sequence FLDGIDKAQEDHEKYHSNWRAM (SEQ ID NO: 16) should provide less, if any protection. The amino acid sequence FLDGIDKAQEEHEKYHSNWRAM (SEQ ID NO: 15) is expected to contain/an HLA-B*4402 restricted CTL epitope.

(SEQ ID NO: 17)

(ii) GKWSKSSMVGWPAVRERMRRAEP
and HLA-C*0701

A change in amino acid residue from the consensus amino acid of proline (P) at position 14 of the protein nef occurs more often than expected by chance in individuals with HLA-C*0701 than in patients without this HLA allele (odds ratio=6.8, P-value=0.0001; after adjustment for other HLA alleles). Furthermore, HLA-C*0701 positive individuals with an amino acid other than proline at position 14 of nef have increased viral load compared to those HLA-C*0701 positive patients with a proline at this position. Hence a therapeutic that included the consensus amino acid of proline at position 14 would offer protection to HLA-C*0701 positive patients compared to the most common other amino acid seen in these patients at this position of a serine (S). Therefore, the amino acid sequence GKWSKSSMVGWPAVRERMRRAEP (SEQ ID NO: 17) should provide protection to HLA-C*0701 positive patients if included in a therapeutic while the sequence GKWSKSSMVGWSAVRERMRRAEP (SEQ ID NO: 18) should provide less, if any protection. The amino acid sequence GKWSKSSMVGWPAVRERMRRAEP (SEQ ID NO: 17) is expected to contain an HLA-C*0701 restricted CTL epitope.

(SEQ ID NO: 19)

(iii) AQEEEEVGFPVRPQVPLRPMTYK
and HLA-B*0702

A change in amino acid residue from the consensus amino acid of arginine (R) at position 71 of the protein nef occurs more often than expected by chance in individuals with HLA-B*0702 than in patients without this HLA allele (odds ratio=19.4, P-value=0.0002; after adjustment for other HLA alleles). Furthermore, HLA-B*0702. positive individuals with an amino acid other than arginine at position 71 of nef have increased viral load compared to those HLA-B*0702 positive patients with a arginine at this position. Hence a therapeutic that included the consensus amino acid of arginine at position 71 would offer protection to HLA-B*0702 positive patients compared to the most common other amino acid seen in these patients at this position of a lysine (K). Therefore, the amino acid sequence AQEEEEVGFPVRPQVPLRPMTYK (SEQ ID NO: 19) should provide protection to HLA-B*0702 positive patients if included in a therapeutic while the sequence AQEEEEVGFPVKPQVPLRPMTYK (SEQ ID NO: 20) should provide less, if any protection. The amino acid sequence AQEEEEVGFPVRPQVPLRPMTYK (SEQ ID NO: 19) is expected to contain an HLA-B*0702 restricted CTL epitope.

(SEQ ID NO: 21)

(iv) SFRFGEETTTPSQKQEPIDKENY
and HLA-B*4402

A change in amino acid residue from the consensus amino acid of serine (S) at position 25 of the protein p6 occurs more often than expected by chance in individuals with HLA-B*4402 than in patients without this HLA allele (odds ratio=66.3, P-value=0.0003; after adjustment for other HLA alleles). Furthermore, HLA-B*4402 positive individuals with an amino acid other than serine at position 25 of p6 have increased viral load compared to those HLA-B*4402 positive patients with a serine at this position. Hence a therapeutic that included the consensus amino acid of serine at position 25 would offer protection to HLA-B*4402 positive patients compared to the most common other amino acid seen in these patients at this position of a proline (P). Therefore, the amino acid sequence SFRFGEETTTPSQKQEPIDKENY (SEQ ID NO: 21) should provide protection to HLA-B*4402 positive patients if included in a therapeutic while the sequence SFRFGEETTTPPQKQEPIDKENY (SEQ ID NO: 22) should provide less, if any protection. The amino acid sequence SFRFGEETTTPSQKQEPIDKENY (SEQ ID NO: 21) is expected to contain an HLA-B*4402 restricted CTL epitope.

(SEQ ID NO: 23)

(v) RIGCQHSRIGIIRQRRARNGASR
and HLA-DRB1-0701

A change in amino acid residue from the consensus amino acid of threonine (T) at position 84 of the protein vpr occurs less often than expected by chance in individuals with HLA-DRB1-0701 than in patients without this HLA allele (odds ratio=0.03, P-value=0.0005; after adjustment for other HLA alleles). Furthermore, HLA-DRB1-0701 positive individuals with an amino acid other than threonine at position 84 of vpr have decreased viral load compared to those HLA-DRB1-0701 positive patients with a threonine at this position. Hence a therapeutic that included the most common amino acid other than the consensus amino acid found in patients with HLA-DRB1-0701 of an isoleucine (I) at position 84 would offer protection to HLA-DRB1-0701 positive patients compared to the consensus amino acid of a threonine. Therefore, the amino acid sequence RIGCQHSRIGITRQRRARNGASR (SEQ ID NO: 23) should provide protection to HLA-DRB1-0701 positive patients if included in a therapeutic while the sequence RIGCQHSRIGITRQRRARNGASR (SEQ ID NO: 24) should provide less, if any protection. The amino acid sequence RIGCQHSRIGIIRQRRARNGASR (SEQ ID NO: 23) is expected to contain an HLA-DRB1-0701 restricted CTL epitope.

(SEQ ID NO: 25)

(vi) KTIHTDNGSNFTSTTVKAACWWA
and HLA-C*0501

A change in amino acid residue from the consensus amino acid of threonine (T) at position 122 of the protein integrase occurs more often than expected by chance in individuals with HLA-C*0501 than in patients without this HLA allele (odds ratio=17.2, P-value=0.0005; after adjustment for other HLA alleles). Furthermore, HLA-C*0501 positive individuals with an amino acid other than threonine at position 122 of integrase have increased viral load compared to those HLA-C*0501 positive patients with a threonine at this position. Hence a therapeutic that included the consensus amino acid of threonine at position 122 would offer protection to HLA-C*0501 positive patients compared to the most common other amino acid seen in these patients at this position of a isoleucine (I). Therefore, the amino acid sequence KTIHTDNGSNFTSTTVKAACWWA (SEQ ID NO: 25) should provide protection to HLA-C*0501 positive patients if included in a therapeutic while the sequence KTIHTDNGSNFISTTVKAACWWA (SEQ ID NO: 26) should provide less, if any protection. The amino acid sequence KTIHTDNGSNFTSTTVKAACWWA (SEQ ID NO: 25) is expected to contain an HLA-C*0501 restricted CTL epitope.

(SEQ ID NO: 27)

(vii) TGADDTVLEEMNLPGRWKPKMIG and HLA-DRB1-1302

A change in amino acid residue from the consensus amino acid of asparagine (N) at position 37 of the protein protease occurs more often than expected by chance in individuals with HLA-DRB1-1302 than in patients without this HLA allele (odds ratio=20.0, P-value=0.0006; after adjustment for other HLA alleles). Furthermore, HLA-DRB1-1302 positive individuals with an amino acid other than asparagine at position 37 of protease have increased viral load compared to those HLA-DRB1-1302 positive patients with an asparagine at this position. Hence a therapeutic that included the consensus amino acid of asparagine at position 37 would offer protection to HLA-DRB1-1302 positive patients compared to the most common other amino acid seen in these patients at this position of a serine (S). Therefore, the amino acid sequence TGADDTVLEEMNLPGRWKPKMIG (SEQ ID NO: 27) should provide protection to HLA-DRB1-1302 positive patients if included in a therapeutic while the sequence TGADDTVLEEMSLPGRWKPKMIG (SEQ ID NO: 28) should provide less, if any protection. The amino acid sequence TGADDTVLEEMNLPGRWKPKMIG (SEQ ID NO: 27) is expected to contain an HLA-C*0701 restricted CTL epitope.

(SEQ ID NO: 29)

(viii) GEETTTPSQKQEPIDKENYPLAS and HLA-A*2402

A change in amino acid residue from the consensus amino acid of glutamate (E) at position 29 of the protein p6 occurs more often than expected by chance in individuals with HLA-A*2402 than in patients without this HLA allele (odds ratio=9.4, P-value=0.0008; after adjustment for other HLA alleles). Furthermore, HLA-A*2402 positive individuals with an amino acid other than glutamate at position 29 of p6 have increased viral load compared to those HLA-A*2402 positive patients with a glutamate at this position. Hence a therapeutic that included the consensus amino acid of glutamate at position 29 would offer protection to HLA-A*2402 positive patients compared to the most common other amino acid seen in these patients at this position of a glycine (G). Therefore, the amino acid sequence GEETTTPSQKQEPIDKENYPLAS (SEQ ID NO: 29) should provide protection to HLA-A*2402 positive patients if included in a therapeutic while the sequence GEETTTPSQKQGPIDKENYPLAS (SEQ ID NO: 30) should provide less, if any protection. The amino acid sequence GEETTTPSQKQEPIDKENYPLAS (SEQ ID NO: 29) is expected to contain an HLA-A*2402 restricted CTL epitope.

(SEQ ID NO: 31)

(ix) WPVKTIHTDNGSNFTSTTVKAAC and HLA-B*4402

A change in amino acid residue from the consensus amino acid of serine (S) at position 119 of the protein integrase occurs more often than expected by chance in individuals with HLA-B*4402 than in patients without this HLA allele (odds ratio=273.6, P-value=0.0009; after adjustment for other HLA alleles). Furthermore, HLA-B*4402 positive individuals with an amino acid other than serine at position 119 of integrease have increased viral load compared to those HLA-B*4402 positive patients with a serine at this position. Hence a therapeutic that included the consensus amino acid of serine at position 119 would offer protection to HLA-B*4402 positive patients compared to the most common other amino acid seen in these patients at this position of a proline (P).

Therefore, the amino acid sequence WPVKTIHTDNGSNFTSTTVKAAC (SEQ ID NO: 31) should provide protection to HLA-B*4402 positive patients if included in a therapeutic while the sequence WPVKTIHTDNGPNFTSTTVKAAC (SEQ ID NO: 32) should provide less, if any protection. The amino acid sequence WPVKTIHTDNGSNFTSTTVKAAC (SEQ ID NO: 31) is expected to contain an HLA-B*4402 restricted CTL epitope.

(SEQ ID NO: 33)

(x) MQRGNFRNQRKTVKCFNCGK and HLA-B*1801

A change in amino acid residue from the consensus amino acid of glutamine (Q) at position 9 of the protein p7 occurs more often than expected by chance in individuals with HLA-B*1801 than in patients without this HLA allele (odds ratio=30.5, P-value=0.0010; after adjustment for other HLA alleles). Furthermore, HLA-B*1801 positive individuals with an amino acid other than glutamine at position 9 of p7 have increased viral load compared to those HLA-B*1801 positive patients with a glutamine at this position. Hence a therapeutic that included the consensus amino acid of glutamine at position 9 would offer protection to HLA-B*1801 positive patients compared to the most common other amino acid seen in these patients at this position of a proline (P). Therefore, the amino acid sequence MQRGNFRNQRKTVKCFNCGK (SEQ ID NO: 33) would provide protection to HLA-B*1801 positive patients if included in a therapeutic while the sequence MQRGNFRNPRKTVKCFNCGK (SEQ ID NO: 34) should provide less, if any protection. The amino acid sequence MQRGNFRNQRKTVKCFNCGK (SEQ ID NO: 33) is expected to contain an HLA-B*1801 restricted CTL epitope.

By following the procedures disclosed herein a therapeutic composition of matter comprising one or more of the above sequences can be prepared and is expected to be useful for treating HIV infected individuals with the identified specific HLA association.

Identified amino acid sequences can be obtained either commercially or prepared following well known techniques known in the field of protein chemistry and which are eluded to herein.

### EXAMPLE 8

Clinical Trial for HIV Vaccine—Evaluation of CD8 and CD4 T-Cell Responses Directed Against Mutated Epitopes in HIV-1 Positive Individuals with Drug Resistant Virus

This example describes a protocol to facilitate an HIV vaccine clinical trial. The various elements of conducting a clinical trial, including patient treatment and monitoring, will be known to those of skill in the art in light of the present disclosure. Generally, the clinical study of the therapeutic described herein should consist of the administration of one or more of the polypeptides herein described, to human subjects to evaluate safety and cellular, antibody, humoral and other clinical responses. The following information is being presented as a general guideline for use in HIV vaccine clinical trials. Information regarding design of clinical trials can also be obtained in the American Foundation for AIDS Research's HIV Experimental Vaccine Directory, Vol 1, No. 2, June 1998.

The subject must be healthy as defined by a normal physical exam and normal laboratory parameters as defined by the WHO for participants in clinical studies. Subjects must be able to understand and sign an informed consent. Subjects must also have a normal total white blood cell count, lymphocyte, granulocyte and platelet count as well hemoglobin and hematocrit. Subjects must has normal values of the following parameters: urinalysis; BUN; creatinine; bilirubin; SGOT; SGPT; alkaline phosphatase; calcium; glucose; CPK; CD4+ cell count; and normal serum immunoglobulin profile.

The following are exclusion criteria: HIV-seropositive status; Active drug or alcohol abuse; inability to give an informed consent; medication which may affect immune function with the exception of low dose of nonprescription-strength NSAIDS, aspirin, or acetaminophen for acute conditions such as headache or trauma; any condition which in the opinion of the principal investigator, might interfere with completion of the study or evaluation of the results.

The study will be double blind randomized. The placebo will be the vaccine solution without the inactivated viral particles. Subjects will be assigned randomly to one of the vaccine routes described above.

Dose Range: Doses dose is in the range of about 1.0 μg to about 50 mg, followed by boosting dosages of from about 1.0 μg to 50 mg, will be studied for clinical safety and immunogenicity.

Administration: For each dose to be tested, the schedule may consist of administration of a dose on days 0, 30, 60, and a booster dose at 180 days.

Route of administration will be intramuscular. Additional routes of administration may include: subcutaneous; oral; intrarectal; intravaginal; intranasal/intramuscular, intrarectal/intramuscular; intranasal/subcutaneous; intrarectal/subcutaneous.

Number of Subjects Per Route of Administration: There will be 12 subjects per route of administration per dose level. Of the 12 subjects 8 will receive the vaccine and 4 will receive a solution without inactivated viral particles.

The endpoint for clinical safety is no evidence of alteration of the clinical, immunological or laboratory parameters. The endpoint for immunological efficacy is seroconversion with production of an effective cellular, humoral and antibody response against HIV. The effective immunological cellular response can be studied by using cytotoxic T lymphocytes responses against different clades of HIV.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### EXAMPLE 9

### Diagnostic Use for Evaluating HIV Adaptation to HLA-Restricted Immune Responses in HIV Infected Patients with Specific HLA Types

The information obtained from the aforementioned population based analyses and as illustrated in FIGS. 1 to 4 and Table 6 can be used to determine the specific amino acid residues to be sequenced in a patient depending on their HLA type to evaluate the extent to which their HIV virus has escaped HLA-restricted immune responses. This information may be used to individualize and guide the timing and type of treatment to be used. In general treatment should aim to prevent further HIV escape from or adaptation to HLA-restricted immune responses.

According to this example the sequences identified in Example 6 are synthesised using standard protein synthesis techniques known in the art. Such techniques are described in Sambrook et al., Molecular Cloning: A Laboratory Manual, Second Edition, Cold Spring Harbor Laboratory Press, Cold Spring Harbor, New York (1989); Ausubel, F., Brent, R., Kingston, R. E., Moore, D. D., Seidman, J. G., Smith, J. A., Struhl, K. Current protocols in molecular biology. Greene Publishing Associates/Wiley Intersciences, New York.

Once the proteins have been sequences they are then conveniently used generate antibodies according to the methodology described first in Kohler and Milstein, Nature, 256:495-497 (1975).

Antibodies prepared by the above methodology are then employed in an ELISA assay as described in Chapter 11 of Ausubel, the disclosure is herein incorporated by reference.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method for determining the influence of variation in host genes on selection of microorganisms with protein substitutions, comprising the steps of:
(a) selecting a population of patients or animals infected with a particular microorganism and typing all individuals of the cohort for at least one selected intrinsic polymorphic marker involved in the host's response to the presence of the microorganism;
(b) identifying and determining at least part of a polynucleotide and or polypeptide sequence in the microorganism in a sufficient number of individuals from each type identified in step (a) in the cohort;
(c) determining the consensus (i.e. most frequent) amino acid across the cohort at each residue position of the sequence analysed in step (b);
(d) comparing the data obtained in step (a) and in step (b) to determine how the host polymorphic sequence (s) in step (a) increase or decrease the probability of a microorganism polymorphism at the first amino acid residue of interest in sequence determined in step (b); and
(e) repeating step (d) for each amino acid identified in step (b) and comparing the data obtained.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. A method for identifying the influence and interaction of variation in host polymorphic marker sequences and a second variable such as a therapeutic drug or vaccine on selection of micro-organisms with particular amino acid variants, which method comprises the steps of:
(a) selecting a population of patients or animals infected with a micro-organism some of which have received the second variable as part of a treatment regime for the micro-organism and typing the individuals of the cohort for at least one selected intrinsic host polymorphic marker sequence (s) involved in the host's response to the presence of the micro-organism;
(b) identifying and determining in a sufficient number of individuals from each type in the cohort part or all of a polynucleotide and or polypeptide sequence in the micro-organism that is a potential or known target for the second variable, before and during exposure to the second variable and in similar but untreated individuals at a similar interval
(c) determine whether a change (“mutation”) has occurred at each residue of the sequence examined in step (b) between the time points identified in step (b);
(d) comparing the data obtained in step (a) and the effect of presence or absence of exposure to the second variable in treated and untreated sequences and the data obtained in step (c) to determine how the polymorphic sequence (s) in step (a) and exposure to the second variable may affect the probability of mutation of the first amino acid residue of interest in step (c);
(e) repeating step (d) for each amino acid in the sequence determined in step (c).

14. A method for determining the influence and interaction of variation in host polymorphic marker sequences and therapeutic drugs on selection of microorganisms with particular amino acid variants, which method comprises the steps of:
(a) selecting a population of patients or animals infected with a microorganism some of whom have received at least one pharmaceutical (s) intended for the treatment of the presence of the microorganism and typing the individuals of the cohort for at least one selected intrinsic host polymorphic marker sequence (s) involved in the host's response to the presence of the microorganism;
(b) identifying and determining part or all of a polynucleotide or polypeptide sequence in the microorganism that is a potential target of the pharmaceutical in each treated individual of the cohort before and during exposure to the pharmaceutical and in similar but untreated individuals at a similar interval;
(c) determining whether a change (“mutation”) has occurred at each residue of the sequence examined in step (b) between the time points identified in step (b);
(d) comparing the data obtained in step (a) and the effect of presence or absence of exposure to the pharmaceutical between treated and untreated sequences and the data obtained in step (c) to determine how the polymorphic sequences in step (a) and pharmaceutical exposure may affect the mutation of the first amino acid residue of interest in step (c); and
(e) repeat step (d) for each amino acid in the sequence determined in step (c).

15. A method comprising the steps of:
a. HLA sequencing a population of hosts infected with HIV;
b. sequencing the whole or part of the dominant HIV species in each patient;
c. defining the consensus sequence for HIV by determining the most common amino acid residue at each residue position of the virus;
d. at each organism residue:
(i) determine for each individual (patient) whether the [HIV] amino acid residue of interest is the same (“non mutated) or different (“mutated”) compared to the consensus residue;
(ii) perform a multivariate regression analysis with mutated amino acids being assigned a value of (1) or non-mutated amino acids being assigned a value of (0) as the outcome of interest; and
(iii) Examine for suitable potential explanatory co-variate in the multivariate model looking for associations with the outcome of interest.

16. (canceled)

17. (canceled)

18. (canceled)

19. (canceled)

20. A method to design therapeutics capable of inducing a specific T-cell response in a patient, that method comprising the steps of:
(a) carrying out the method of claim 1 as described above; and
(b) analysing the data to identify polymorphisms arising in a virus population as a result of infection of that population, which polymorphisms are HLA associated; and
(c) preparing a therapeutic which includes the polymorphism identified in step (b).

21. A method to identify T cell epitopes, that method comprising the steps:
(a) carrying out the method of claim 1 as described above; and
(b) analysing the data to identify the polymorphism frequency arising in a virus population as a result of infection of that population, which polymorphisms are HLA associated.

22. A method for designing a vaccine to prevent or delay the emergence of drug resistance in patients treated with a particular drug specific for a micro-organism, wherein the drug affects the replication of the microorganism at the nucleotide or amino acid level, which method comprises the steps of:
(a) carrying out the method of claim 1 as described above; and
(b) analysing the data to identify the polymorphism frequency arising in a virus population in an infected individual which has been treated with an antiretroviral drug, wherein the polymorphism frequency is determined over the nucleotide or amino acid sequence regions where the drug is active in the micro-organism; and (c) designing one or more therapeutics which facilitate a T-cell response to cells that contain a virus population displaying one or more of the identified polymorphisms.

23. A method to design therapeutics according to claim 20, wherein the polypeptide sequence employed in the method is selected from the group consisting of SEQ ID NO: 2 to 10, 11, 13, 15, 17, 19, 21, 23, 25, 27, 29, 31 or 33.

24. A method to identify T cell epitopes according to claim 21, wherein the polypeptide sequence employed in the method is selected from the group consisting of SEQ ID NO: 2 to 10, 11, 13, 15, 17, 19, 21, 23, 25, 27, 29, 31 or 33.

25. A method for designing a vaccine to prevent or delay the emergence of drug resistance in patients according to claim 22, wherein the polypeptide sequence employed in the method is selected from the group consisting of SEQ ID NO: 2 to 10, 11, 13, 15, 17, 19, 21, 23, 25, 27, 29, 31 or 33.

26. A therapeutic prepared according to the method of claim 20.

27. A method according to claim 20 wherein the therapeutic prepared according to step (c) includes a vector construct capable of expressing an amino acid sequence in a patient, said sequence displaying the polymorphism identified in step (b) in a manner capable of inducing or eliciting a T-cell response in said patient.

28. A method according to claim 22 wherein the therapeutic designed according to step (c) includes a vector construct capable of expressing an amino acid sequence in a patient, said sequence displaying the polymorphism identified in step (b) in a manner capable of inducing or eliciting a T-cell response in said patient.

29. A polypeptide sequence of SEQ ID NO:13.

30. A therapeutic composition comprising amino acid SEQ ID NO:13.

31. A vector construct capable of expressing an amino acid sequence in a patient comprising a nucleotide sequence capable of expressing an amino acid sequence comprising SEQ ID NO:13.

32. A method for identifying at least an amino acid in an amino acid sequence in a microorganism that is resistant to or prone to variation induced by at least a polymorphic marker sequence in an individual, said method comprising the steps of:
(a) selecting a population of individuals infected with a microorganism and identifying a polymorphic marker type present in each member of the population, wherein the marker(s) is associated with each individual's response to the presence of the microorganism;
(b) sequencing, from a selection of the individual in each type selected in step (a), a polypeptide sequence expressed by the microorganism, and separating those sequences according to each marker type identified in step (a);
(c) determining, from the sequences identified in step (b), a consensus amino acid sequence, by assigning the most common amino acid in the population at each amino acid position;
(d) determining, within each type identified in step (a), the probability of an amino acid polymorphism at each amino acid in the consensus sequence by comparing the first amino acid in the consensus amino acid sequence obtained in step (c) against the first amino acid in each sequence identified in step (b);
(e) repeating step (d) for each amino acid in the consensus sequence identified in step (c); and
(f) correlating the results in step (a) with the results in step (e) to identify statistically significant associations between each amino acid in the consensus sequence and the polymorphic marker sequence, wherein said association indicates at least an amino acid that is resistant to or prone to variation induced by at least a polymorphic marker sequence in an individual.

33. A method according to claim 32 wherein the method is used to identify regions of an amino acid sequence that are resistant to or prone to variation induced by at least a polymorphic marker sequence in an individual.

34. A method according to claim 32 wherein univariate or multivariate statistical analysis is employed in step (d).

35. A method according to claim 32 wherein the polymorphic marker sequence is an amino acid sequence.

36. A method according to claim 32 wherein the polymorphic marker sequence is a nucleotide sequence.

37. A method according to claim 32 wherein multiple logistic regression analysis is used in step (d), wherein in said analysis the data obtained in step (a) is employed as the explanatory co-variable and the data obtained in step (b) as the outcome variable in the model.

38. A method according to claim 37 wherein a polymorphism is ascribed a one value and no polymorphism is ascribed an alternate value as the outcome of interest.

39. A method according to claim 32 wherein the polymorphic marker is a HLA marker.

40. A method according to claim 39 wherein the HLA marker is selected from the group consisting of: HLA class IA, HLA class IB, HLA class IC, HLA Class II DR, HLA Class II DQ.

41. A method according to claim 32 wherein the marker selected in step (a) is a receptor or other protein actively engaged in host-microorganism interaction.

42. A method according to claim 32 wherein the marker selected in step (a) is a chemokine receptor.

43. A method according to claim 32, wherein the marker selected in step (a) is the CCR5 receptor involved in HIV binding.

44. A method according to claim 32 wherein the microorganisms is selected from the group: HIV, HCV or HBV.

45. A method for determining whether a polymorphism in a microorganism polypeptide sequence that is the result of a cytotoxic T lymphocyte escape mutation, said method comprising the steps of:
(a) selecting a population of individuals infected with a microorganism and identifying the HLA markers present in each member of the population, wherein the HLA markers are associated with each individual's response to the presence of the microorganism;
(b) sequencing, from a selection of the individuals in each type selected in step (a), a part of the polypeptide sequence expressed by the micro-organism that contains the polymorphism, and separating those sequences according to the HLA marker types identified in step (a);
(c) determining, from the sequences identified in step (b), a consensus amino acid sequence, which includes the polymorphism, by assigning the most common amino acid in the population at each amino acid position;
(d) determining, for each HLA marker identified in step (a), the probability of the amino acid polymorphism by comparing in the consensus amino acid sequence obtained in step (c) against the polymorphic amino acid in each sequence identified in step (b); and
(e) correlating the results in step (a) with the results in step (d) to identify whether there is a positive or negative association between the HLA alleles and the polymorphic amino acid, wherein said association indicates the polymorphism is HLA allele-specific.

46. A method for identifying the location of cytotoxic T lymphocyte epitopes, said method comprising the steps of:
(a) selecting a population of individuals infected with a microorganism and identifying the HLA markers present in each member of the population, wherein the HLA markers are associated with each individual's response to the presence of the microorganism;
(b) sequencing, from a selection of the individual in each type selected in step (a), a polypeptide sequence expressed by the microorganism, and separating those sequences according to the HLA marker types identified in step (a);
(c) determining, from the sequences identified in step (b), a consensus amino acid sequence, by assigning the most common amino acid in the population at each amino acid position;
(d) determining, for each HLA marker identified in step (a) that has a univariate association of about P<0.1 with a polymorphism, the probability of an amino acid polymorphism at each amino acid in the consensus sequence by comparing the first amino acid in the consensus amino acid sequence obtained in step (c) against the first amino acid in each sequence identified in step (b);
(e) repeating step (d) for each amino acid in the consensus sequence identified in step (c); and
(f) correlating the results in step (a) with the results in step (e) to identify statistically significant positive or negative associations between the HLA alleles and the consensus amino acid sequence, wherein said association indicates a possible location for a CTL epitope.

47. A method for identifying the effect that a therapeutic drug and a polymorphic marker sequence in an individual have on the mutation of amino acids in a microorganism, said method comprising the steps of:
(a) selecting:
(i) a population of individuals infected with a microorganism which have also received a therapeutic agent as treatment for the microorganism and identifying at least a polymorphic marker type present in each member of the population, wherein the marker is associated with each individual's response to the presence of the microorganism; and
(ii) selecting a population of individuals infected with a microorganism which have not received a therapeutic agent as treatment for the microorganism and identifying the same polymorphic marker type as selected in step (a) present in each member of the population, wherein the marker is associated with each individual's response to the presence of the microorganism;
(b) sequencing:
(i) from a selection of the individual in each type selected in step (a)(i), a polynucleotide and or polypeptide sequence from the microorganism that is a potential or known target for the therapeutic drug;
(ii) from a selection of the individual in each type selected in step (a)(ii), a polynucleotide and or polypeptide sequence from the microorganism that corresponds to the sequence that is sequenced in step (b)(i);
(c) comparing the sequences in step (b)(i) against the sequences in step (b)(ii) to determine whether a polynucleotide and or polypeptide sequence mutation has arisen at each residue in the sequences examined in step (b);
(d) determining, within each marker type identified in step (a)(i) and step (a)(ii), the probability of an sequence polymorphism at each mutation identified in step (c); and
(e) comparing the data obtained in step (a) with the data obtained in step (d) to identify statistically significant associations between both of the polymorphic marker sequence and the therapeutic agent and the identified mutations, wherein the association indicates mutations that a microorganism will develop to escape recognition by a the therapeutic agent in a marker type.

48. A method comprising the steps of:
(a) HLA sequencing a population of individuals infected with HIV;
(b) sequencing at least a part of the dominant HIV species in each individual that was HLA sequenced in step (a);
(c) identifying a consensus sequence for the sequences that are sequenced in step (b) by determining the most common amino acid residue from all the sequences identified at each residue position of the virus;
(d) At each residue:
(i) identifying for each individual whether the HIV amino acid residue of interest is the non-mutated or mutated compared to the consensus residue;
(ii) performing multivariate regression statistical analysis on the data obtained in steps (a) to (c) wherein mutated amino acids being assigned one value, non-mutated amino acids are assigned an alternate value as the outcome of interest and the consensus sequence identified in step (c) is used as a reference sequence; and
(e) analysing the data obtained in step (d)(ii) to identify statistically significant associations, wherein said association indicates the probability of a mutation in the HIV polynucleotide sequence or amino acid sequence as a consequence of the presence of the HLA marker examined.

49. A method according to claim 48 wherein in step (d) (ii) the explanatory co-variate is an HLA allele of an individual HLA sequenced in step (a).

50. A method comprising the steps of:
(a) HLA sequencing:
(i) a population of individuals infected with HIV, wherein the individuals have been treated with a therapeutic agent that is active against a nucleotide or amino acid sequence of HIV;
(ii) a population of individuals infected with HIV, wherein the individuals have not been treated with a therapeutic agent active against a nucleotide or amino acid sequence of HIV;
(b) sequencing at least a part of the dominant HIV species in each individual that was HLA sequenced in steps (a)(i) and (a)(ii);
(c) identifying a consensus sequence for the sequences that are sequenced in step (b) by determining the most common amino acid residue from all the sequences identified at each residue position of the virus;
(d) At each residue:
(i) identifying for each individual whether the HIV amino acid residue of interest is the non-mutated or mutated compared to the consensus residue;
(ii) performing multivariate regression statistical analysis on the data obtained in steps (a) to (c) wherein mutated amino acids being assigned one value, non-mutated amino acids are assigned an alternate value as the outcome of interest and the consensus sequence identified in step (c) is used as a reference sequence; and
(e) analysing the data obtained in step (d)(ii) to identify statistically significant associations, wherein said association indicates the probability of a mutation in the HIV polynucleotide sequence or amino acid sequence as a consequence of the presence of the HLA marker examined.

51. A method according to claim 50 wherein in step (d) (ii) the explanatory co-variate is the therapeutic agent of interest.

52. A method according to claim 51 wherein the therapeutic drug is a reverse transcriptase inhibitor anti-retroviral drug or a protease inhibitor.

53. A method according to claim 1 wherein the microorganism sequence examined is selected from the group consisting of: SEQ ID NO: 1 to 14.


SEQUENCE LISTING (35 sequences)

SEQ ID NO: 1 - protein, 163 aa, HIV
FAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTKILEPFRKQNPDIVIYQYMDDLYVGSDLEIGQHRTKIEELRQHLLRWGFTTPDKKHQK

SEQ ID NO: 2 - protein, 500 aa, HIV
MGARASVLSGGELDRWEKIRLRPGGKKKYKLKHIVWASRELERFAVNPGLLETSEGCRQILGQLQPSLQTGSEELKSLYNTVATLYCVHQRIEVKDTKEALDKIEEEQNKSKKKAQQAAADTGNSSQVSQNYPIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKTVKCFNCGKEGHIARNCRAPRKKGCWKCGKEGHQMKDCTERQANFLGKIWPSHKGRPGNFLQSRPEPTAPPEESFRFGEETTTPSQKQEPIDKELYPLASLRSLFGNDPSSQ

SEQ ID NO: 3 - protein, 1003 aa, HIV
FFRENLAFPQGKAREFSSEQTRANSPTRRELQVWGEDNNSTSEAGADRQGTVSFSFPQITLWQRPLVTIKIGGQLKEALLDTGADDTVLEEMNLPGRWKPKMIGGIGGFIKVRQYDQIIIEICGHKAIGTVLVGPTPVNIIGRNLLTQLGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDKDFRKYTAFTIPSINNETPGIRYQYNVLPQGWKGSPAIFQSSMTKILEPFRKQNPDIVIYQYMDDLYVGSDLEIGQHRTKIEELRQHLLKWGFTTPDKKHQKEPPFLWMGYELHPDKWTVQPIVLPEKDSWTVNDIQKLVGKLNWASQIYAGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKIATESIVIWGKTPKFKLPIQKETWEAWWTEYWQATWIPEWEFVNTPPLVKLWYQLEKEPIVGAETFYVDGAANRETKLGKAGYVTDRGRQKVVSLTDTTNQKTELQAIHLALQDSGLEVNIVTDSQYALGIIQAQPDKSESELVSQIIEQLIKKEKVYLAWVPAHKGIGGNEQVDKLVSAGIRKVLFLDGIDKAQEEHEKYHSNWRAMASDFNLPPVVAKEIVASCDKCQLKGEAMHGQVDCSPGIWQLDCTHLEGKIILVAVHVASGYIEAEVIPAETGQETAYFLLKLAGRWPVKTIHTDNGSNFTSTTVKAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRDPLWKGPAKLLWKGEGAVVIQDNSDIKVVPRRKAKIIRDYGKQMAGDDCVASRQDED

SEQ ID NO: 4 - protein, 192 aa, HIV
MENRWQVMIVWQVDRMRIRTWKSLVKHHMYISKKAKGWFYRHHYESTHPRISSEVHIPLGDAKLVITTYWGLHTGERDWHLGQGVSIEWRKRRYSTQVDPDLADQLIHLYYFDCFSESAIRNAILGHIVSPRCEYQAGHNKVGSLQYLALAALITPKKIKPPLPSVTKLTEDRWNKPQKTKGHRGSHTMNGH

SEQ ID NO: 5 - protein, 96 aa, HIV
MEQAPEDQGPQREPYNEWTLELLEELKSEAVRHFPRIWLHGLGQHIYETYGDTWAGVEAIIRILQQLLFIHFRIGCQHSRIGITRQRRARNGASRS

SEQ ID NO: 6 - protein, 101 aa, HIV
MEPVDPRLEPWKHPGSQPKTACTNCYCKKCCFHCQVCFIKKGLGISYGRKKRRQRRRAPQDSQTHQVSLSKQPASQPRGDPTGPKESKKKVERETETDPVD

SEQ ID NO: 7 - protein, 116 aa, HIV
MAGRSGDSDEELLKTVRLIKFLYQSNPPPSPEGTRQARRNRRRRWRERQRQIRSISGWILSTYLGRPAEPVPLQLPPLERLTLDCNEDCGTSGTQGVGSPQILVESPAVLESGTKE

SEQ ID NO: 8 - protein, 82 aa, HIV
MQPLEILAIVALVVAAIIAIVVWTIVFIEYRKILRQRKIDRLIDRIRERAEDSGNESEGEESALVEMGVEMGHHAPWDVDDL

SEQ ID NO: 9 - protein, 856 aa, HIV
MRVKGNNQHLWKWGWKWGTMLLGMLMICSATEKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLNNDTNTNNTSGSNNMEKGEIKNCSFNITTSIRDKMQKEYALFYKLDVVPIDNDNTSYRLISCNTSVITQACPKVSFEPIPIHYCAPAGFAILKCNDKKFNGTGPCTNVSTVQCTHGIRPVVSTQLLLNGSLAEEEVVIRSENFTNNAKTIIVQLNESVEINCTRPNNNTRKSISIHIGPGRAFYATGEIGDIRQAHCNISRAEWNNTLKQIVKKLREQFGKNKTIVFNQSSGGDPEIVMHSFNCGGEFFYCNTTQLFNSTWNNSTWNTEESNNTEGNETITLPCRIKQIINMWQEVGKAMYAPPIRGQIRCSSNITGLLLTRDGGNNNNKTETFRPGGGDMRDNWRSELYKYKVVKIEPLGVAPTKAKRRVVQREKRAVGIGAMFLGFLGAAGSTMGAASITLTVQARQLLSGIVQQQNNLLRAIEAQQHLLQLTVWGIKQLQARVLAVERYLKDQQLLGIWGCSGKLICTTAVPWNTSWSNKSLNKIWDNMTWMEWEKEINNYTGIIYNLIEESQNQQEKNEQELLELDKWASLWNWFDISKWLWYIKIFIMIVGGLIGLRIVFAVLSIVNRVRQGYSPLSFQTHLPTPRGPDRPEGIEEEGGERDRDRSSRLVDGFLAIIWDDLRSLCLFSYHRLRDLLLIVTRIVELLGRRGWEILKYWWNLLQYWSQELKNSAVSLLNATAIAVAEGTDRIIEVVQRACRAILHIPRRIRQGVERALL

SEQ ID NO: 10 - protein, 206 aa, HIV
MGGKWSKSSMVGWPAVRERMRRAEPAADGVGAVSRDLEKHGAITSSNTAATNADCAWLEAQEEEEVGFPVRPQVPLRPMTYKGALDLSFFLKEKGGLEGLIYSQKRQDILDLWVYHTQGYFPDWQNYTPGPGIRYPLTFGWCFKLVPVEPEKVEEANEGENNSLLHPMSQHGMDDPEREVLMWKFDSRLAFRHMARELHPEYYKDC

SEQ ID NO: 11 - protein, 138 aa, HIV
PQITLWQRPIVTIKIGGQLREALLDTGADNTVLEEMNLPGRWKPKIIGGVGGFIKVRQYDQIPIEICGHKAIGTVLVGPTPANIIGRNLMTQIGCTLNFGRWKPKMIVGIGGLIKVRQYDQLVGPTPVNVIGRNLLTQ

SEQ ID NO: 12 - protein, 138 aa, HIV
PQITLWQRPLVTIKIGGQLKEALLDTGADDTVLEEMNLPGRWKPKMIGGIGGFIKVRQYDQIPIEICGHKAIGTVLVGPTPVNIIGRNLLTQIGCTLNFGRWKPKMIGGIGGFIKVRQYDQLVGPTPVNIIGRNLLTQ

SEQ ID NO: 13 - protein, 203 aa, HIV
LVEICTELEKEGKISTPVFAIKRKDSTRWRKLVDFDIVIYQYVDDLYVGSHLLKWGFYTPDKKHQICTEMEKDGKISKIGAIKKKDSDKWRKVVDFRELNQLGIPHPGGLKKNKSVTVLDVGDAYFSIPLDKDFRYQYNVLPMGWKGSPAQNPDIVICQYMDDLYVASDLEIGQHRTKIEELRQHLWKWGFFTPDQKHQKEPP

SEQ ID NO: 14 - protein, 203 aa, HIV
LVEICTEMEKEGKISTPVFAIKKKDSTKWRKLVDFDIVIYQYMDDLYVGSHLLKWGFTTPDKKHQICTEMEKEGKISKIGAIKKKDSTKWRKLVDFRELNQLGIPHPAGLKKKKSVTVLDVGDAYFSVPLDKDFRYQYNVLPQGWKGSPAQNPDIVIYQYMDDLYVGSDLEIGQHRTKIEELRQHLLKWGFTTPDKKHQKEPP

SEQ ID NO: 15 - protein, 22 aa, hiv
FLDGIDKAQEEHEKYHSNWRAM

SEQ ID NO: 16 - protein, 22 aa, HIV
FLDGIDKAQEDHEKYHSNWRAM

SEQ ID NO: 17 - protein, 23 aa, HIV
GKWSKSSMVGWPAVRERMRRAEP

SEQ ID NO: 18 - protein, 23 aa, HIV
GKWSKSSMVGWPAVRERMRRAEP

SEQ ID NO: 19 - protein, 23 aa, HIV
AQEEEEVGFPVRPQVPLRPMTYK

SEQ ID NO: 20 - protein, 23 aa, HIV
AQEEEEVGFPVKPQVPLRPMTYK

SEQ ID NO: 21 - protein, 23 aa, HIV
AQEEEEVGFPVKPQVPLRPMTYK

SEQ ID NO: 22 - protein, 23 aa, HIV
SFRFGEETTTPSQKQEPIDKENY

SEQ ID NO: 23 - protein, 23 aa, HIV
SFRFGEETTTPPQKQEPIDKENY

SEQ ID NO: 24 - protein, 23 aa, HIV
RIGCQHSRIGIIRQRRARNGASR

SEQ ID NO: 25 - protein, 23 aa, HIV
RIGCQHSRIGITRQRRARNGASR

SEQ ID NO: 26 - protein, 23 aa, HIV
KTIHTDNGSNFTSTTVKAACWWA

SEQ ID NO: 27 - protein, 23 aa, HIV
KTIHTDNGSNFISTTVKAACWWA

SEQ ID NO: 28 - protein, 23 aa, HIV
TGADDTVLEEMNLPGRWKPKMIG

SEQ ID NO: 29 - protein, 23 aa, HIV
TGADDTVLEEMSLPGRWKPKMIG

SEQ ID NO: 30 - protein, 23 aa, HIV
GEETTTPSQKQEPIDKENYPLAS

SEQ ID NO: 31 - protein, 23 aa, HIV
GEETTTPSQKQGPIDKENYPLAS

SEQ ID NO: 32 - protein, 23 aa, HIV
WPVKTIHTDNGSNFTSTTVKAAC

SEQ ID NO: 33 - protein, 23 aa, HIV
WPVKTIHTDNGPNFTSTTVKAAC

SEQ ID NO: 34 - protein, 20 aa, HIV
MQRGNFRNQRKTVKCFNCGK

SEQ ID NO: 35 - protein, 20 aa, HIV
MQRGNFRNPRKTVKCFNCGK